(19) United States
(12) Patent Application Publication
(10) Pub. No.: US 2026/0053903 A1
(43) Pub. Date: Feb. 26, 2026

(54) COMPOSITIONS AND METHODS FOR TARGETING DENDRITIC CELL LECTINS

(71) Applicants: MASSACHUSETTS INSTITUTE OF TECHNOLOGY, Cambridge, MA (US); GEORGIA TECH RESEARCH CORPORATION, Atlanta, GA (US)

(72) Inventors: Valerie LENSCH, Cambridge, MA (US); Laura L. KIESSLING, Cambridge, MA (US); Mohammad Murshid ALAM, Cambridge, MA (US); M. G. FINN, Atlanta, GA (US); Robert HINCAPIE, Atlanta, GA (US)

(21) Appl. No.: 19/104,231

(22) PCT Filed: Aug. 21, 2023
(86) PCT No.: PCT/US2023/072578

§ 371 Date: Feb. 17, 2025

Related U.S. Application Data
(60) Provisional application No. 63/399,590, filed on Aug. 19, 2022.

(51) Int. Cl. A61K 39/00; A61P 37/04; C12N 7/00

(52) U.S. Cl. CPC A61K 39/0011; A61K 39/001102; A61K 39/001129; A61P 37/04; C12N 7/00; A61K 2039/5258; A61K 2039/53; C12N 2795/18023; C12N 2795/18034

(57) ABSTRACT

Compositions and methods of glycosylated virus-like particles (VLPs) displaying user-defined antigens and optionally encapsidating TLR ligands for targeting dendritic cell lectins have been developed. The VLPs employ ligands for a DC lectin e.g., DC-SIGN to activate dendritic cells (DC) and drive proliferation of antigen-specific CD8 and CD4-T cells specific for a user-defined antigen, such as a tumor antigens. In some forms, the compositions include aryl-mannose ligands to effectively generate DC-mediated TH-1 T cell responses to the user-defined antigen.

### CROSS-REFERENCE TO RELATED APPLICATIONS

This application claims the benefit of and priority to U.S. Provisional Application No. 63/399,590 filed Aug. 19, 2022, which is hereby incorporated by reference in its entirety.

### STATEMENT REGARDING FEDERALLY SPONSORED RESEARCH OR DEVELOPMENT

This invention was made with government support under R01AI055258 awarded by The National Institute of Allergy and Infectious Diseases. The U.S. government has certain rights in the invention.

### REFERENCE TO SEQUENCE LISTING

The Sequence Listing submitted as a text file named “MIT_24570J_ST26,” created on Aug. 21, 2023, and having a size of 11,287 bytes is hereby incorporated by reference pursuant to 37 C.F.R. § 1.52(e)(5).

### FIELD OF THE INVENTION

The invention generally relates to the field of molecular systems for delivery of antigen, and more specifically, to virus-like particles for the targeted delivery of antigens to dendritic cells to prevent or treat diseases and/or conditions.

### BACKGROUND OF THE INVENTION

Vaccination remains the most effective method of preventing infectious diseases. The World Health Organization (WHO) reports that licensed vaccines are currently available to prevent or contribute to the prevention and control of twenty-five infections (World Health Organization, Global Vaccine Action Plan 2011-2020, Geneva, 2012).

Cancer vaccines rely on priming T cells to recognize tumor (neo)antigens and mediate tumor-specific cell killing. Typically, T cell priming requires tumor antigen-presentation by dendritic cells (DCs), which are the key initiator of tumor-specific/anti-tumor immune responses. DCs are powerful immune cells that can initiate and modulate adaptive immune responses. As professional antigen-presenting cells, DCs internalize and degrade pathogens and present the processed peptide epitopes to T cells. Through this pathway, DCs can prime T cells to both internal and external threats including viruses, parasites, and tumors. T cell receptors recognize the bound antigen on either major histocompatibility complex (MHC) class I or MHC class II molecules, resulting in the stimulation of cytotoxic T cells and helper T cells, respectively. Additionally, in response to pathogen engagement, DCs secrete specific cytokines that activate different components of cellular, humoral, or tolerogenic immunity. Due to their importance in modulating immunity via T cell polarization, targeting DCs is essential to the development of effective cancer vaccines. However, it remains a challenge to engineer vaccines that elicit the cellular immune responses necessary for effective therapeutic cancer vaccines.

Current approaches to the delivery of vaccine antigens often rely on technology that is limited in rapid production capability and associated engineering parameters to influence the type, duration, and potency of an immune response. While some vaccine strategies effectively employ protein antigen, custom peptides often lack immunogenic potential and require extensive adjuvating and may be most useful as in vitro screening tools.

There exists a need for improved systems for effectively directing antigen-specific immune responses in a subject.

Therefore, it is an object of the invention to provide compositions and methods for the targeted delivery of antigen(s) to professional antigen presenting cells (APC) within a subject, using delivery vehicles that direct specific types of immunity to the antigen(s) in the subject with minimal or no cytotoxicity.

It is a further object of the invention to provide methods and compositions for the simultaneous delivery of two or more antigens to professional antigen presenting cells, to generate the same, or different forms of immunity to the antigen(s) within the recipient subject.

It is a further object of the invention to provide methods and compositions for the delivery of therapeutic molecules into the cells of subject.

### SUMMARY OF THE INVENTION

Compositions that selectively target and activate antigen-presenting cells (APCs) to stimulate antigen-specific immunity in a subject have been developed. Compositions including virus-like particles (“VLPs”) for the targeted delivery of antigens to professional antigen-presenting cells (APCs) in a subject are provided.

Typically, the VLPs are formed of a multiplicity of viral capsid proteins modified to express one or more peptide antigens for display at the surface of the VLP. A preferred VLP capsid is formed from the Leviviridae PP7 capsid protein.

The VLPs efficiently drive proliferation of antigen-specific CD8+ T and/or CD4+ T cells against intracellular antigen, and potentiate specific antibody responses against the antigens. The VLPs include one or more ligands for a receptor or lectin expressed at the surface of APCs such as Dendritic Cells (DCs) and one or more antigens at the surface of the VLP. In some forms, the VLPs include or encapsulate one or more ligands for Toll-Like Receptors (TLRs). In further forms, the VLPs also include or encapsulate one or more additional therapeutic, prophylactic or diagnostic agents for targeted delivery to APCs such as DCs.

The antigen(s) to be delivered will typically be a polynucleotide, protein or peptide. In some embodiments, the VLPs include includes one, two, or more different antigens (e.g., two different antigenic peptide sequences, etc., also referred to herein as different “species” of antigen) that direct the same or different immune responses in a subject. In some embodiments, two or more antigens different antigens are all tethered to the VLP in separate physical locations. Additionally or alternatively, the two or more antigens can be tethered to the VLP in the same location, e.g. in the form of a single fusion protein including both or more antigen sequences, optionally separated by a linker (e.g., a cleavable linker). In some embodiments, the compositions and methods include two or more VLPs each including only one antigen that are direct the same or different immune responses in a subject. In some embodiments, the VLPs and/or compositions that include two or more different antigens include at least one MHC class I antigen in combination with at least one MHC class II antigen.

Vaccines including the VLPs for the delivery of antigens and a pharmaceutically acceptable excipient for administration to a subject, and methods of using the VLPs and vaccines and other compositions formed therefrom are also provided. In some embodiments, the vaccines, compositions, and methods include VLPs including two or more antigens.

### BRIEF DESCRIPTION OF THE DRAWINGS

FIGS. 1A-1E illustrate VLP-Man for cancer vaccine. FIG. 1A is a schematic representation showing VLPs (●) bearing MHC I epitopes and MHC II epitopes of tumor antigens (Δ) and mannose ligands (◯) binding to the tetrameric lectin DC-SIGN on the surface of a dendritic cell (DC), and subsequent release of cytokines (°) by the DC and presentation of the MHC I and MHC II epitopes to naive T cells, which in turn mature into cytotoxic (CD8+) T cells (CTL) and T-helper (CD4+) T cells (TH-1 cell), respectively, which together produce cytokines (IL-2/IFN-γ) to provide effective antigen specific anti-tumor immunity. FIG. 1B is a schematic representation of the chemical synthesis of phenyl mannose ligands for attachment to the surface of VLPs. Briefly, arylmannoside alkyne, N-(4-(((2R,3S,4S,5S,6R)-3,4,5-trihydroxy-6-(hydroxymethyl)tetra-hydro-2H-pyran-2-yl)oxy)phenethyl)pent-4-ynamide, Man (mannose) is synthesized from addition of acetylated D-mannose with the ligand core in presence of boron trifluoride etherate (BF₃). FIG. 1C is a further schematic of VLP-Man engagement of DC-SIGN and TLR7 on DCs for antigen presentation and T cell priming. FIG. 1D is an illustration of design of VLPs surface-functionalized with tumor antigens OvaI (SIINFEKL (SEQ ID NO:10)) and OvaII (ISQAVHAAHAEINEAGR) (SEQ ID NO:11)), control pentaerythritol or mannose ligands, and encapsulated ssRNAs. FIG. 1E is an illustration of the functionalization of surface accessible lysines using NHS-ester chemistry to incorporate an azide handle followed by click chemistry to attach tumor antigens or ligands. Steps (i, ii): 0.1 M potassium phosphate buffer, pH 7.4; room temperature, 2 h; step (iii): tris(3 hydroxypropyltriazolylmethyl)amine (THPTA), CuSO4, sodium ascorbate, aminoguanidine, 0.1 M potassium phosphate, pH 7.0, 50° C., 1 h.

FIGS. 2A-2E illustrate that Man-OvaI/II co-engages DC-SIGN and TLR7 and activates DCs. FIG. 2A is a line graph. moDCs were incubated with 8 nM AF647-labeled VLPs over 30 min and uptake of VLP by moDCs was measured by flow cytometry. FIG. 2B is a bar graph showing mean fluorescence intensity of AF647-labeled VLPs in moDCs indicated at 30 minutes. FIG. 2C is a bar graph. moDCs were pre-treated with blocking antibodies against PRRs for 20 min and then incubated with 8 nM Man-OvaI/II for 15 min, and uptake of VLPs was assessed by flow cytometry. Relative uptake was calculated by normalization against a control sample without pre-treatment of blocking antibodies. FIGS. 2D and 2E are each a series of bar graphs showing activation of moDCs by VLPs. moDCs were incubated with 8 nM VLPs for 24 h, and the expression levels of activation surface markers CD40, CD83, CD86, and CD80 were measured by flow cytometry. Data represent mean+/− standard error of the mean (s.e.m.) from a representative experiment (n=3). Statistical analysis was performed using one-way ANOVA with Bonferroni's multiple comparisons test. *P<0.1, **P<0.01, ***P<0.001, ****P<0.0001.

FIGS. 3A-3C illustrate Man-OvaI/II induces Th1-associated cytokine secretion. FIG. 3A is a bar graph. moDCs were treated with VLPs for 32 h and supernatants were collected for cytokine analysis. FIG. 3B is a bar graph. moDCs were pre-treated with blocking antibodies against DC-SIGN for 20 min followed by stimulation with PE-OvaI/II or Man-OvaI/II for 32 h, and TNF-α secretion was measured. FIG. 3C is a bar graph. moDCs were treated with Man-OvaI/II or the corresponding ssRNA-depleted VLP, and TNF-α secretion was measured. Data represent mean+/−s.e.m. from a representative experiment (n=3). Statistical analysis was performed by one-way ANOVA with Bonferroni's multiple comparisons test. *P<0.1, **P<0.01, ***P<0.001, ****P<0.0001.

FIGS. 4A-4D illustrate VLPs traffic to lymph nodes for uptake by immune cells. FIG. 4A is a series of IVIS images. C57BL/6 mice were immunized with AF647-labelled VLPs and organs were isolated for quantification. FIG. 4B is a bar graph showing quantification of average radiant efficiency within each organ of interest FIG. 4C is a bar graph showing LN-resident cells were analyzed for VLP uptake by flow cytometry. Percentage of DCs positive for AF647. FIG. 4D is a bar graphs showing MFI of AF647 among DCs. Data represent mean+/−s.e.m. from a representative experiment (n=3 (FIG. 4B) and n=6 (FIGS. 4C-4D). Statistical analysis was performed by unpaired two-tailed Student's t-test. ****P<0.0001.

FIGS. 5A-5J illustrate mannosylated VLP conjugation with tumor antigens induces specific CD4+ and CD8+ T cells. FIG. 5A is a schematic representation of VLPs with Ova antigens. FIG. 5B is a timeline showing C57/BL6 mice were inoculated subcutaneously (s.c.) with 5×10⁵ B16F10-OVA cells on day 0 and treated with VLPs and 2′3′-cGAMP (s.c.) and anti-PD-1 intraperitoneally (i.p.) on days 3, 9, and 15. FIG. 5C is a series of bar graphs showing percentages of CD4+ and CD8+ T cells in the spleens were analyzed on day 21. FIG. 5D is a series of bar graphs showing IFN-γ and TNF-α-secreting CD4+ T cells in the spleen were measured following restimulation with OVA(323-339). FIG. 5E is a series of bar graphs showing IFN-γ and TNF-α-secreting CD4+ T cells in the spleen were measured following restimulation with OVA(257-264). FIG. 5F is a series of bar graphs showing percentages of CD4+ and CD8+ T cells in the tumors were analyzed on day 21. FIG. 5G is a series of bar graphs showing percentages of CD4+ and CD8+ among CD3+ T cells within the tumor. FIG. 5H is a line graph showing mean tumor growth curves. FIG. 5I is a dot plot showing tumor volumes on day 21. FIG. 5J is plot showing survival curves. Data represent mean+/−s.e.m. from a representative experiment (n=5 (FIGS. 5C-5G) and n=8 (FIGS. 5H-5J). Statistical analysis was performed by one-way ANOVA with Bonferroni's multiple comparisons test or by log-rank (Mantel-Cox) test for the survival analysis. *P<0.1, **P<0.01, ***P<0.001, ****P<0.0001.

FIGS. 6A-6N illustrate that Man-OvaI/II induces tumor antigen-specific T cells and exerts anti-tumor response. FIG. 6A is a timeline showing C57/BL6 mice were inoculated subcutaneously (s.c.) with 5×10⁵ B16F10-OVA cells on day 0 and treated with VLPs and 2′3′-cGAMP (s.c.) and anti-PD-1 intraperitoneally (i.p.) on days 3, 9, and 15. FIG. 6B is a line graph showing mean tumor growth curves. FIG. 6C is a dot plot showing tumor volumes on day 21. FIG. 6D is a plot showing survival curves. FIG. 6E is a series of bar graphs showing percentages of CD4+ and CD8+ T cells in the spleens were analyzed on day 21. FIG. 6F is a series of bar graphs showing IFN-γ and TNF-α-secreting CD4+ T cells in the spleen were measured following restimulation with OVA(323-339). FIG. 6G is a series of bar graphs showing IFN-γ and TNF-α-secreting CD8+ T cells in the spleen were measured following restimulation with OVA(257-264). FIG. 6H is a series of bar graphs showing percentages of CD4+ and CD8+ T cells in the tumors were analyzed on day 21. FIG. 6I is a series of bar graphs showing percentages of CD4+ and CD8+ among CD3+ T cells within the tumor. FIG. 6J is a timeline showing surviving mice were re-challenged with 5×10⁴ B16F10-OVA cells on day 28. FIG. 6K is plot showing tumor growth curves. FIGS. 6L and 6M are bar graphs showing OVA323-specific antibody profiles were analyzed by ELISA measurements from serum of mice immunized with PE-OVAI/II (FIG. 6L) or VLP-Man (FIG. 6M). FIG. 6N is a bar graphs showing IgG2c:IgG1 antibody ratio for mice immunized with VLPs. Data represent mean+/−s.e.m. from a representative experiment (n=8 (FIGS. 6B-6D) and n=5 (FIGS. 6E-6I) and n=4 (FIGS. 6J-6K) and n=5 (FIGS. 6L-6N)). Statistical analysis was performed by one-way ANOVA with Bonferroni's multiple comparisons test or by log-rank (Mantel-Cox) test for the survival analysis. *P<0.1, **P<0.01, ***P<0.001, ****P<0.0001.

FIGS. 7A-7G illustrate Man-OvaI/II has self-adjuvanting capacity. FIG. 7A is a timeline showing C57/BL6 mice were inoculated subcutaneously (s.c.) with 5×10⁵ B16F10-OVA cells on day 0 and treated with VLPs and 2′3′-cGAMP (s.c.) and anti-PD-1 intraperitoneally (i.p.) on days 3, 9, and 15. FIG. 7B is a plot showing mean tumor growth curves. FIG. 7C is a dot plot showing tumor volumes on day 21. FIG. 7D is a plot showing survival curves. FIG. 7E is a series of bar graphs showing IFN-γ and TNF-α-secreting CD4+ T cells in the spleen were measured following restimulation with OVA(323-339). FIG. 7F is a series of bar graphs showing IFN-γ and TNF-α-secreting CD8+ T cells in the spleen were measured following restimulation with OVA(257-264). FIG. 7G is a series of bar graphs showing percentages of CD4+ and CD8+ T cells in the tumors were analyzed on day 21. Data represent mean+/−s.e.m. from a representative experiment (n=8 (FIGS. 7B-7D) and n=5 (FIG. 7E-7G)). Statistical analysis was performed by one-way ANOVA with Bonferroni's multiple comparisons test or by log-rank (Mantel-Cox) test for the survival analysis. *P<0.1, **P<0.01, ***P<0.001, ****P<0.0001.

### DETAILED DESCRIPTION OF THE INVENTION

### I. Definitions

The term “host cell” refers to prokaryotic and eukaryotic cells into which a recombinant expression vector can be introduced.

The term “Aryl” refers to C5-C26-membered aromatic or fused aromatic ring systems. Examples of aromatic groups are benzene, naphthalene, anthracene, phenanthrene, chrysene, pyrene, corannulene, coronene, etc.

The term “Alkyl” refers to saturated or unsaturated aliphatic groups, including straight-chain alkyl, alkenyl, or alkynyl groups, branched-chain alkyl, alkenyl, or alkynyl groups, cycloalkyl, cycloalkenyl, or cycloalkynyl (alicyclic) groups, alkyl substituted cycloalkyl, cycloalkenyl, or cycloalkynyl groups, and cycloalkyl substituted alkyl, alkenyl, or alkynyl groups. Unless otherwise indicated, a straight chain or branched chain alkyl has 30 or fewer carbon atoms in its backbone (e.g., C1-C30 for straight chain, C3-C30 for branched chain), more preferably 20 or fewer carbon atoms, more preferably 12 or fewer carbon atoms, and most preferably 8 or fewer carbon atoms. Likewise, preferred cycloalkyls have from 3-10 carbon atoms in their ring structure, and more preferably have 5, 6 or 7 carbons in the ring structure. The alkyl groups can also be substituted with one or more groups including, but not limited to, halogen, hydroxy, amino, thio, ether, ester, carboxy, oxo, and aldehyde groups. The alkyl groups may also contain one or more heteroatoms. “Lower alkyl”, means 1-6 carbons, preferably 1-5 carbons, more preferably 1-4 carbons, most preferably 1-3 carbons.

The terms “amine” and “amino” are art-recognized and refer to both unsubstituted and substituted amines, e.g., a moiety that can be represented by the general formula:

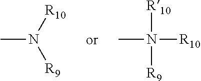

wherein, R₉, R₁₀, and R′₁₀ each independently represent a hydrogen, an alkyl, an alkenyl, —(CH₂)ₘ—R₈ or R₉ and R₁₀ taken together with the N atom to which they are attached complete a heterocycle having from 4 to 8 atoms in the ring structure; R₈ represents an aryl, a cycloalkyl, a cycloalkenyl, a heterocycle or a polycycle; and m is zero or an integer in the range of 1 to 8. In preferred embodiments, only one of R₉ or R₁₀ can be a carbonyl, e.g., R₉, R₁₀ and the nitrogen together do not form an imide. In still more preferred embodiments, the term “amine” does not encompass amides, e.g., wherein one of R₉ and R₁₀ represents a carbonyl. In even more preferred embodiments, R₉ and R₁₀ (and optionally R′₁₀) each independently represent a hydrogen, an alkyl or cycloalkyl, an alkenyl or cycloalkenyl, or alkynyl. Thus, the term “alkylamine” refers to an amine group, as defined above, having a substituted (as described above for alkyl) or unsubstituted alkyl attached thereto, i.e., at least one of R₉ and R₁₀ is an alkyl group.

The term “amide” is art-recognized as an amino-substituted carbonyl and includes a moiety that can be represented by the general formula:

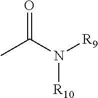

wherein, R₉ and R₁₀ are as defined above.

The term “specifically binds” to a target refers to a binding reaction which is determinative of the presence of the molecule in the presence of a heterogeneous population of other biologics.

The term “vector” refers to a nucleic acid molecule or polynucleotide, such as a replicating RNA, plasmid, phage, or cosmid, into which another nucleic acid sequence segment may be inserted so as to bring about the replication of the inserted segment. The described vectors can be expression vectors.

The term “nucleic acid” refers to any natural or synthetic linear and sequential arrays of nucleotides and nucleosides, for example, DNA including complementary DNA (cDNA), replicating RNA (repRNA), messenger RNA (mRNA), small interfering RNA (siRNA), transfer RNA (tRNA), microRNA (miRNA), guide strand RNA (sgRNA), polynucleotides, oligo-nucleotides, oligo-nucleosides and derivatives thereof. Such nucleic acids may be collectively referred to as “constructs,” or “plasmids. Representative examples of the nucleic acids include bacterial plasmid vectors including expression, cloning, cosmid and transformation vectors such as, but not limited to, viral vectors, vectors derived from bacteriophage nucleic acid, and synthetic oligonucleotides like chemically synthesized DNA or RNA. The term “nucleic acid” further includes modified or derivatized nucleotides and nucleosides such as, but not limited to, halogenated nucleotides such as, but not only, 5-bromouracil, and derivatized nucleotides such as biotin-labeled nucleotides.

The term “gene” or “genes” refers to isolated or modified nucleic acid sequences, including both RNA and DNA, that encode genetic information for the synthesis of a whole RNA, a whole protein, or any portion of such whole RNA or whole protein. Genes that are not naturally part of a particular organism's genome are referred to as “foreign genes”, “heterologous genes” or “exogenous genes” and genes that are naturally a part of a particular organism's genome are referred to as “endogenous genes”. The term “gene” as used with reference to genomic DNA includes intervening, non-coding regions as well as regulatory regions and can include 5′ and 3′ ends.

The term “expressed” or “expression” refers to the transcription from DNA to an RNA nucleic acid molecule at least complementary in part to a region of one of the two nucleic acid strands of the gene. The term “expressed” or “expression” also refers to the translation from said RNA nucleic acid molecule to give a protein or polypeptide or a portion thereof.

The term “antigen” refers to any substance (e.g., peptide, protein, nuclei acid, lipid, small molecule, such as a moiety expressed by or otherwise associated with a pathogen or cancerous or pre-cancerous cell) that serves as a target for the receptors of an adaptive immune response. The antigen may be a structural component of a pathogen, cancerous or pre-cancerous cell.

The term “pathogen” refers to an organism or other entity that causes a disease. For example, pathogens can be prions, viruses, prokaryotes such as bacteria, eukaryotes such as protozoa and fungi. A pathogen can be the source of an antigen to which an adaptive immune response can be generated.

The term “polypeptide” includes proteins and fragments thereof. Polypeptides are described as amino acid residue sequences. Those sequences are written left to right in the direction from the amino (N) to the carboxyl (C) terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

The term “antibody” is used in the broadest sense unless clearly indicated otherwise. Therefore, an “antibody” can be naturally occurring or man-made, such as monoclonal antibodies produced by conventional hybridoma technology. Antibodies include monoclonal and polyclonal antibodies as well as fragments containing the antigen-binding domain and/or one or more complementarity determining regions of these antibodies. “Antibody” refers to any form of antibody or antigen binding fragment thereof and includes monoclonal antibodies (including full length monoclonal antibodies), polyclonal antibodies, multi-specific antibodies (e.g., bi-specific antibodies), and antibody fragments.

The terms “individual,” “individual,” “subject,” and “patient” are used interchangeably, and refer to a mammal, including, but not limited to, humans, rodents, such as mice and rats, and other laboratory animals.

The term “biocompatible” refers to one or more materials that are neither themselves toxic to the host (e.g., an animal or human), nor degrade (if the polymer degrades) at a rate that produces monomeric or oligomeric subunits or other byproducts at toxic concentrations in the host.

The term “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions.

The terms “Virus-like Particle” and “VLP” are used interchangeably to refer to small particles that contain certain proteins from the outer coat of a virus and can be constructed to present these proteins as antigens on their coat. VLPs are self-assembled protein structures that can efficiently deliver antigens and have demonstrated the ability to elicit humoral immune responses. Typically, VLPs lack the viral components that are required for virus replication and thus represent a highly attenuated, replication-incompetent form of a virus. VLPs are non-replicating viral shells, derived from any of several viruses. VLPs can display one or more molecules at the outer surface of the particle and can encapsulate one or more molecules within the particle. For example, in some forms, VLPs include antigens associated with a pathogen or cancer and one or more lectin-binding ligands attached to the surface and one or more nucleic acids encapsulated within the VLP. VLPs can thereby elicit an immune response to the corresponding pathogen or cancer when administered to a subject.

The terms “dendritic cell-specific intercellular adhesion molecule-3-grabbing nonintegrin”, “DC-SIGN”, and CD209 refer to the mannose- and fucose-binding protein with UniProt Database Accession ID NO: Q9NNX6. DC-SIGN is a pathogen-recognition receptor expressed on the surface of immature dendritic cells (DCs) and involved in initiation of primary immune response. DC-SIGN serves as a receptor for viruses, bacteria, yeast, and parasites. Importantly, DC-SIGN can induce the immunostimulatory cellular TH1/CTL-type immune response that is desired for an anti-tumor response. As used herein, “optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range, from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these embodiments are explicitly disclosed.

Every compound disclosed herein is intended to be and should be considered to be specifically disclosed herein. Further, every subgroup that can be identified within this disclosure is intended to be and should be considered to be specifically disclosed herein. As a result, it is specifically contemplated that any compound, or subgroup of compounds can be either specifically included for or excluded from use or included in or excluded from a list of compounds.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular polypeptide is disclosed and discussed and a number of modifications that can be made to a number of polypeptides are discussed, specifically contemplated is each and every combination and permutation of polypeptides and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

### II. Compositions for Targeted Delivery of Antigens to Antigen Presenting Cells

The development of effective cancer vaccines remains slowed by the challenge of inducing tumor-specific cellular immunity. Tumor antigen presentation by dendritic cells (DCs), the primary initiators of anti-tumor immune responses, is important for in vivo T cell priming. The examples below illustrate the design and testing of an exemplary DC lectin-targeted virus-like particle (VLP) vaccine that delivers programmable peptide antigens to induce tumor-specific cellular immunity. Co-engagement of the lectin DC-SIGN by VLP-presented synthetic mannose ligands and of Toll-like receptor 7 by encapsulated single-stranded RNA induced DC maturation and secretion of Th-1 type cytokines. Vaccination with glycosylated VLPs enabled robust antigen presentation by DCs, significantly increasing tumor-specific CD4+ and CD8+ T cells compared to non-targeted VLPs. These T cells were capable of tumor infiltration and led to prolonged survival in a murine melanoma model. Glycosylated VLPs are self-adjuvanting and can efficiently activate DCs that generate robust anti-tumor responses, thus offering a framework for advancing cancer immunotherapies.

Thus, compositions for the delivery of antigens to professional antigen presenting cells (APCs), such as Dendritic Cells (DCs) are provided. The compositions typically include a particle, such as a virus-like particle (VLP) that displays one or more antigen(s) and one or more agents targeting APCs at the outer surface of the particle. Typically, the targeting agents are ligands for one or more binding partners present at the surface of DCs, such as lectins. Therefore, VLPs including one or more lectin-binding moieties and one or more antigens for targeting DCs are provided. In some forms, the VLPs include and/or encapsulate one or more additional active agents.

DC lectin-targeting VLPs functionalized with MHC I and MHC II peptide epitopes are described. As described in examples and the associated figures and their descriptions, it has been demonstrated that DC lectin targeting successfully induces cell mediated immune responses/T cell priming. As shown in the experiments below, DC lectin-targeted virus-like particles (VLPs) were engineered to display both DC-SIGN glycans and tumor/neoantigens for cross presentation to induce tumor antigen specific cellular immune responses that are important for anti-cancer immunity. These DC lectin-targeted VLPs induce a robust tumor antigen-specific Th1/CTL-type immune response. The data reported in the Figures emphasizes the importance of DC activation in anti-tumor immunity.

In preferred forms the VLPs include one or more TLR ligands. For example, in some forms, the VLPs encapsulate nucleic acids that act as TLR agonists.

In some embodiments, Virus Like Particle (VLP) for antigen-specific activation of dendritic cells, include
(a) a DC-SIGN ligand; and
(b) one or more peptide antigen(s),
wherein the DC-SIGN ligand and the antigen(s) are present at the outer surface of the synthetic particle are provided. An exemplary VLP capsid is formed from a multiplicity of Leviviridae PP7 capsid proteins. In some forms, the VLP is decorated with at least two antigen species.

The compositions target and activate Dendritic Cells (DC). DCs are responsible for initiating antigen-specific immune responses and are the master regulators of the immune response. DCs link the microbial sensing features of the innate immune system to the exquisite specificity of the adaptive response. DCs are efficient at antigen presentation and generate the right type of T cells in response to a given pathogen. Therefore, DCs help guide the immune system to respond to foreign antigens while avoiding the generation of autoimmune responses to self and are paradoxically important in cancer, generating both immunity and tolerance.

DCs act to communicate the presence of pathogens to the adaptive immune system to initiate long lasting, antigen-specific responses. DCs carefully marshal the proteolytic apparatus in both the endosomal-lysosomal system (cathepsins and other lysosomal hydrolases) as well as in the cytosol (proteasome) and endoplasmic reticulum (ER) to partially degrade pathogen-derived proteins to yield antigenic peptides that in turn are loaded onto MHC class I or class II molecules. The resulting peptide-MHC complexes are transported to the plasma membrane, where they are presented to their cognate T cells that are then activated and induced to proliferate and become potent effector cells (cytotoxic T cells), or cells that assist in the overall progress of the immune responses (helper T cells). T-cell responses are further enhanced and sculpted by the fact that DCs also express ligands (e.g., CD80, CD86) that bind to costimulatory molecules on T cells that act in concert with the peptide-MHC-specific T-cell receptor. DCs also produce a host of stimulatory cytokines, e.g., interleukin12 (IL-12), that are required for optimal T-cell stimulation. Although other cell types (e.g., macrophages, B cells) can present antigen, due to their remarkable efficiency, DCs are uniquely responsible for initiating all antigen-specific immune responses.

DCs can also stimulate B cells directly by presenting intact antigen at the DC surface thereby activating B cells of cognate specificity.

DCs also maintain immune tolerance by ensuring under normal conditions that effector T cells are not produced against the normal or “self” antigens of host cells and tissues. In the absence of infection, i.e., at the steady state, DCs continuously encounter and present self-antigens and nonpathogenic environmental antigens to T cells, so effector T cells are not induced to proliferate, but rather, the production of immunosuppressive regulatory T cells (Tregs) is favored. These “induced” Tregs, as opposed to the naturally occurring Tregs produced in the thymus, also help to prevent unwanted immune responses against noninfectious environmental antigens entering via the gut and airway.

In cancer, as most tumors are far more similar than not to normal cells and are often likely to initiate in the absence of overt inflammation, the ability of DCs to induce peripheral tolerance likely represents a major constraint against the generation of antitumor immunity.

The compositions include one or more molecules that specifically bind DC ligands, such as DC-surface lectins.

Upon encountering pathogen-derived TLR ligands, ligands for intracellular sensors, or proinflammatory molecules, immature DCs are triggered to mature, which converts them in 12 to 24 hours from cells adept at antigen accumulation to cells specialized for T-cell stimulation. After a transient upregulation (presumably to increase the opportunity to capture the newly arrived pathogen), endocytosis is dramatically down regulated. Lysosomes and the antigen-processing machinery are activated, enhancing the efficiency of peptide-MHC production. Ubiquitination of MHC class II and other molecules ceases, allowing peptide-MHC complexes to remain at the cell surface. The DCs are then induced to migrate from tissues to lymphoid organs, in part by upregulating chemokine receptors such as CCR7, begin to efficiently generate peptides that can be loaded stably onto MHC molecules, and upregulate the production of costimulatory ligands and immunostimulatory cytokines. They enter T-cell-rich regions of lymph nodes and begin to stimulate antigen-specific memory or naive T-cell responses.

### A. Virus Like Particles (VLPs)

The compositions generally include a virus-like particle (VLP) as a display platform for the antigen and DC-receptor binding components. VLPs represent a useful format for the multivalent display of glycans and can also carry nucleic acid that can bind and activate the TLRs.

Virus-like particles (VLPs) are self-assembled protein nanostructures composed of multiple copies of one or more structural or envelope coat proteins (CPs). These particles resemble their corresponding natural viruses in structure but lack the genomic cargo necessary for replication.

Similar to their natural precursors, VLPs typically are stable and biocompatible. Those derived from RNA bacteriophages are particularly amenable to high-yield expression and assembly in different systems, including bacteria, yeast, insect cells, and mammalian cells, and they have therefore been employed in a wide variety of biomedical applications. Many such uses derive from their polyvalency: the ability of VLPs to present multiple copies of functional peptides or protein domains to potential binding agents, cells, and tissues.

Previously, VLPs decorated with lectin-binding glycans were used as a platform to facilitate efficient antigen uptake and DC activation (see Alum, et al, ACS Nano. 2021 Jan. 26; 15(1): 309-321, doi:10.1021/acsnano.0c03023, the contents of which are hereby incorporated herein by reference in their entirety).

Typically, a virus-like particle (VLP) includes one or more surface proteins or subunit or other fragment thereof. VLPs are small particles that contain certain proteins from the outer coat of a virus and can be constructed to present these proteins as antigens on their coat. Typically, VLPs lack the viral components that are required for virus replication and thus represent a highly attenuated, replication-incompetent form of a virus. Thus, VLPs can be regarded as non-replicating, viral shells, derived from any of several viruses. The VLP can display a polypeptide (e.g., a spike protein encoded by a disclosed mRNA) that is analogous to that expressed on infectious virus particles and can elicit an immune response to the corresponding virus when administered to a subject.

The presence of VLPs following recombinant expression of viral proteins can be detected using conventional techniques known in the art. For example, the formation of VLPs can be detected by any suitable technique including techniques known in the art for detection of VLPs in a medium include, e.g., electron microscopy techniques, dynamic light scattering (DLS), selective chromatographic separation (e.g., ion exchange, hydrophobic interaction, and/or size exclusion chromatographic separation of the VLPs) and density gradient centrifugation. VLPs can be isolated density gradient centrifugation and identified by characteristic density banding. See, for example, Baker, et al. (1991) Biophys. J. 60:1445-1456; and Hagensee et al. (1994) J. Virol. 68:4503-4505; Vincente, J Invertebr Pathol., 2011; Schneider-Ohrum and Ross, Curr. Top. Microbiol. Immunol., 354: 53073, 2012).

### 1. Sources of Virus Like Particles (VLPs)

The VLPs can be derived from various viruses. In some forms, the VLP is derived from the hepatitis B virus or other virus families including Parvoviridae (e.g., adeno-associated virus), cowpea mosaic virus, flock house virus, and MS223, Retroviridae (e.g., HIV), Flaviviridae (e.g., Hepatitis C virus), bacteriophage Qβ and Leviviridae (e.g., PP7) scaffolds. A general review is provided in Sorensen M R and Thomsen A R, APMIS 115(11):1177-93 (2007) and Guillén et al., Procedia in Vaccinology 2 (2), 128-133 (2010).

VLPs are generally composed of one or more viral proteins, such as, but not limited to, those proteins referred to as capsid, coat, shell, surface and/or envelope proteins, or particle-forming polypeptides derived from these proteins. VLPs can form spontaneously upon recombinant expression of the protein in an appropriate expression system. Exemplary VLPs include the icosahedral Qβ VLP (hydrodynamic diameter ˜36 nm) and the PP7 VLP (hydrodynamic diameter ˜30 nm).

Virus like particles and methods of their production are known and familiar to the person of ordinary skill in the art, and viral proteins from several viruses are known to form VLPs, including human papillomavirus, HIV (Kang et al., Biol. Chem. 380: 353-64 (1999)), Semliki-Forest virus (Notka et al., Biol. Chem. 380: 341-52 (1999)), human polyomavirus (Goldmann et al., J. Virol. 73: 4465-9 (1999)), rotavirus (Jiang et al., Vaccine 17: 1005-13 (1999)), parvovirus (Casal, Biotechnology and Applied Biochemistry, Vol 29, Part 2, pp 141-150 (1999)), canine parvovirus (Hurtado et al., J. Virol. 70: 5422-9 (1996)), hepatitis E virus (Li et al., J. Virol. 71: 7207-13 (1997)), and Newcastle disease virus.

In some forms, the VLP is designed via a two-plasmid expression system for the production of hybrid VLPs composed of a mixture of truncated and extended coat proteins. In preferred forms, the particle will assemble even if every CP has an extension at either end of the truncated CP. Thus, the number of functional moieties presented on each VLP by this technique is a statistical average, and this average varies among different batches even if the expression is performed under the same conditions.

### PP7 VLPs

A preferred VLP is a Leviviridae-derived capsid, such as that produced from the PP7 virus scaffold. PP7 VLPs are described in Zhao, et al., ACS Nano, 13(4): 4443-4454, (2019), doi:10.1021/acsnano.8b09683., the contents of which are hereby incorporated herein by reference in their entirety.

PP7 VLP assembles into homogeneous icosahedral particles with extended peptides on every subunit and allowing for substantial loop insertions into a dimeric CP variant. The three-dimensional structure of the PP7 capsid protein is nearly identical to other Leviviridae, featuring a noncovalent dimer in which interlocked α-helices dominate the particle exterior surface and β-sheet domains are contiguously arranged on the interior surface. In some forms, the VLP adopts a T=4 structure when self-assembled from CP dimers.

In some forms, the PP7 VLP here corresponds to the 127 amino acid natural sequence of the virus reported by Olsthoorn, et al., Virology 1995, 206, 611-625, the contents of which are hereby incorporated by reference in their entirety.

An exemplary amino acid sequence for the PP7 coat protein is:

(SEQ ID NO: 1)
SKTIVLSVGEATRTLTEIQSTADRQIFEEKVGPLVGRLRLTASLRQNG
AKTAYRVNLKLDQADVVDCSTSVCGELPKVRYTQVWSHDVTIVANSTE
ASRKSLYDLTKSLVATSQVEDLVVNLVPLGR.

In some forms, the PP7 coat protein is a dimer, i.e., includes two copies of SEQ ID NO:1. In some forms, the dimer includes a linker region between the two copies. An exemplary linker is the amino acid sequence AYGG (SEQ ID NO:2). For example, in some forms the PP7 dimer includes all or part of the amino acid sequence:

(SEQ ID NO: 3)
SKTIVLSVGEATRTLTEIQSTADRQIFEEKVGPLVGRLRLTASLRQNG
AKTAYRVNLKLDQADVVDCSTSVCGELPKVRYTQVWSHDVTIVANSTE
ASRKSLYDLTKSLVATSQVEDLVVNLVPLGRAYGGSKTIVLSVGEATR
TLTEIQSTADRQIFEEKVGPLVGRLRLTASLRQNGAKTAYRVNLKLDQ
ADVVDCSTSVCGELPKVRYTQVWSHDVTIVANSTEASRKSLYDLTKSL
VATSQVEDLVVNLVPLGR.

Typically, the dimer-based particles include 120 copies of the PP7-PP7 CP, with a hydrodynamic diameter of around 30 nm.

### 2. VLPs Modified for Surface Display

The disclosed VLPs are typically engineered to display one, two, or more additional molecules at their surface. Examples of molecules include one or more antigens, e.g., for inducing an immune response, ligands for enhancing cell targeting and/or internalization.

a. Modified VLPs Displaying Peptides

In some forms, the viral capsid is engineered to include one or more additional polypeptide sequences, for example, for display of the sequences at the surface of the VLP. The VLP must still assemble into a stable structure, such that Inclusion of the additional polypeptide allows for the use of added sequences while maintaining self-assembly.

Therefore, in some forms, the capsid protein dimer is engineered to include one or more additional polypeptides at the carboxyl (C) terminus of the capsid dimer.

In an exemplary form, the PP7 capsid protein dimer is engineered to include one or more additional polypeptides at the carboxyl (C) terminus of SEQ ID NO:3.

In some forms, the dimer includes a linker region between the C terminus and the initiation of the polypeptide. An exemplary linker is the amino acid sequence GGASESGA (SEQ ID NO:4). For example, in some forms the PP7 dimer includes all or part of the amino acid sequence:

(SEQ ID NO: 5)
SKTIVLSVGEATRTLTEIQSTADRQIFEEKVGPLVGRLRLTASLRQNG
AKTAYRVNLKLDQADVVDCSTSVCGELPKVRYTQVWSHDVTIVANSTE
ASRKSLYDLTKSLVATSQVEDLVVNLVPLGRAYGGSKTIVLSVGEATR
TLTEIQSTADRQIFEEKVGPLVGRLRLTASLRQNGAKTAYRVNLKLDQ
ADVVDCSTSVCGELPKVRYTQVWSHDVTIVANSTEASRKSLYDLTKSL
VATSQVEDLVVNLVPLGRGGASESGA.

In other forms, an additional polypeptide is included as a “loop” extension at a part of the capsid that is neither the C or N terminus. In an exemplary form, the additional polypeptide is inserted within the linker sequence between the two copies of PP7 monomer.

Typically, the dimer-based particles are comprised of 120 copies of the PP7-PP7 CP and display 120 functional insertions/extensions.

b. Modified VLPs for Dual Display

In some forms, the viral capsid is engineered to display more than a single protein species at the surface of the VLP. For example, in some forms, the viral capsid is engineered to display two, three, four, five, six, seven, eight, nine ten, or more than ten protein species at the surface of the viral capsid.

Compositions and methods for the simultaneous display of two or more different exogenous peptides on the same particle have been developed. In some forms, a viral capsid protein includes a C-terminal extension and a loop insertion. Preferably, inclusion of the additional polypeptides allows for maintaining self-assembly of the VLP into a stable structure.

Therefore, in some forms, the viral capsid includes two copies of SEQ ID NO:1, as well as a single copy each of SEQ ID NOs:2 and 4, as well as two additional polypeptide sequences.

Typically, the dimer-based particles including two species of additional polypeptides/proteins are comprised of 120 copies of the PP7-PP7 CP and display a total of 240 functional insertions/extensions, such that each species is present in 120 copies.

c. Display Considerations

Experiments using icosahedral Qβ VLP (hydrodynamic diameter ˜36 nm, having 180 copies of a 14 kDa coat protein, each with its N-terminus and three lysine residues exposed on the outer surface, accounting for up to 720 amine conjugation sites), show that ligand density can impact internalization and intracellular signaling. The density of Man and Man3 functionalization on the VLPs was controlled by mixing each of these alkynes with the non-binding PE-alkyne in varying ratios. Each mixture was used in a copper(I)-catalyzed azide-alkyne cycloaddition (CuAAC) reaction in the presence of tris(3 hydroxypropyltriazolylmethyl)amine (THPTA), providing complete conversion of VLP-azides to triazoles. A similar strategy can be used to control the density of ligand and/or antigen display in the disclosed VLP compositions.

Results also show that densely mannosylated particles, Qβ-Man₅₄₀, Qβ-Man3₄₇₅, and Qβ-Man3₂₀₀, were taken upselectively only by the cell line expressing DC-SIGN (Alum, et al, ACS Nano. 2021 Jan. 26; 15(1): 309-321. doi:10.1021/acsnano.0c03023). In contrast, the non-mannosylated Qβ-PE₅₄₀ and the sparsely mannosylated Qβ-Man₉₀ were barely recognized.

Thus, in preferred embodiments, the VLPs present a ligand in an effective number and/or density to facilitate internalization. Preferably, the ligand is also suitable to induce signaling in the target APC cells. In some embodiments, the internalized ligand-receptor complex is insensitive to acidic pH (e.g., in the endosome). In some embodiments, this is due to the dense display of ligand with aryl groups that can engage in hydrophobic and CH-π interactions.

In the Examples below, PP7 VLP display approximately 900 copies of DC-SIGN ligand and 100 copies of antigen, though other ratios and density are also contemplated. Non-limiting examples of polypeptides/proteins and targeting ligands are discussed below.

d. Exemplary Additional

### Polypeptides/Proteins for Display

In some forms, the VLPs are engineered to include one or more peptides, such as peptide antigens, at the surface of the VLP.

In some forms, the VLPs are engineered such that all of the capsid subunits include one or two additional peptides. In other forms, the VLPs are engineered such that only a portion of the total number of the capsid subunits include one or two additional peptides. Therefore, the VLPs can include a desired number of additional polypeptides for display at the surface of the VLP. For example, in some forms, the VLPs include less than 10% of the number of capsid molecules including an additional polypeptide, or more than 10%, such as 20%, 30%, 40%, 50%, 60%, 70%, 80%, 90% or more than 90%, such a 100% of the capsid proteins including one or more additional polypeptides. The density/coverage of the additional peptides can be tuned according to the size and/or characteristics of the additional polypeptide, and the surface density/coverage that is desired.

In some forms, the additional polypeptide sequence is preceded on the peptide extension by a linker sequence. An exemplary linker is a protease cleavable linker.

In preferred forms, the VLPs include one or more additional polypeptides/proteins that are antigens in a subject.

Antigens are compounds that are specifically bound by antibodies or T lymphocyte antigen receptors. They stimulate production of or are recognized by antibodies. Sometimes antigens are part of the host itself and can result in an autoimmune disease when the body attacks the self-antigens.

An immunogen is an antigen (or adduct) that is able to trigger a humoral (innate) or cell-mediated immune response. It first initiates an innate immune response, which then causes the activation of the adaptive immune response. An antigen binds the highly variable immunoreceptor products (B cell receptor or T cell receptor) once these have been generated. Immunogens are those antigens, termed immunogenic, capable of inducing an immune response. Thus, an immunogen is necessarily an antigen, but an antigen may not necessarily be an immunogen. However, unless specifically indicated otherwise, any of the antigens can also be an immunogenic (i.e., an immunogen).

As discussed in more detail below, in some forms, antigens are selected or designed for immune stimulation or immune tolerance, of B-cells and/or T-cells, with or without the context of an MHC complex. In preferred forms, antigens are selected or designed for immune stimulation or immune tolerance, predominantly of T-cells.

In preferred forms, the antigens are those suitable for MHC complex presentation by APC such as dendritic cells. T cells respond to threats in an antigen-specific manner using T cell receptors (TCRs) that recognize short peptide antigens presented on major histocompatibility complex (MHC) proteins. The TCR-peptide-MHC interaction mediated between a T cell and its target cell dictates its function and thereby influences its role in disease. In preferred forms, the antigen is a T cell antigen. In some forms, the T cell antigen is one that requires processing such as proteolytic cleavage by antigen-presenting cell before it can be recognized by the T lymphocytes.

Exemplary peptide antigens include B cell antigens and T cell antigens.

The peptide antigen can be derived from a virus, bacterium, parasite, plant, protozoan, fungus, tissue or transformed cell such as a cancer or leukemic cell and immunogenic component thereof, e.g., cell wall components or molecular components thereof. The antigens can be allergens or environmental antigens or tumor antigens. The antigen can be associated with one or more diseases or conditions such as infectious diseases, autoimmune diseases, and cancer.

Suitable antigens are known in the art and are available from commercial government and scientific sources. The antigens can be purified or partially purified polypeptides derived from tumors or viral or bacterial sources. The antigens can be recombinant polypeptides produced by expressing DNA or mRNA encoding the polypeptide antigen in a heterologous expression system. Antigens can be provided as single antigens or can be provided in combination. Antigens can also be provided as complex mixtures of polypeptides or nucleic acids.

In some forms, the antigen is a viral antigen. A viral antigen can be isolated from any virus. In an exemplary form, the antigen is a natural viral capsid structure or portion thereof, or a composite of a structure of multiple strains of the virus. In some forms, the antigen is a bacterial antigen. Bacterial antigens can originate from any bacteria. In some forms the antigen is a parasite antigen. In some forms, the antigen is an allergen or environmental antigen. Exemplary allergens and environmental antigens, include but are not limited to, an antigen derived from naturally occurring allergens such as pollen allergens (tree-, herb, weed-, and grass pollen allergens), insect allergens (inhalant, saliva and venom allergens), animal hair and dandruff allergens, and food allergens. In some forms, the antigen is a self-antigen such as in immune tolerance applications for auto-immune or related disorders such as Multiple Sclerosis. In some forms, the antigen is a tumor antigen. Exemplary tumor antigens include a tumor-associated or tumor-specific antigen.

In further forms, the antigens are those in an approved vaccines that are designed to elicit an immune response to protect again a particular pathogen. Vaccines can elicit a response based ono whole-pathogen vaccines such as inactivated viruses, live-attenuated viruses, and chimeric vaccine; subunit vaccines such as protein subunit vaccines, peptide vaccines, virus-like particles (VLPs), and recombinant proteins; and nucleic acid-based vaccines such as DNA plasmid vaccines, mRNA vaccines, and recombinant vector vaccines utilizing viral expression vectors. Exemplary vaccines include Adenovirus Type 4 and Type 7 Vaccine, ERVEBO® (Ebola Zaire Vaccine, Live), DENGVAXIA® (Dengue Tetravalent Vaccine, Live), DAPTACEL® (Diphtheria and Tetanus Toxoids and Acellular Pertussis Vaccine), M-M-R II® (Measles, Mumps, and Rubella Virus Vaccine Live), TRUMENBA® (Meningococcal Group B Vaccine), POLIOVAX® (Poliovirus Vaccine Inactivated), IMOVAX® (Rabies Vaccine), RABAVERT® (Rabies Vaccine), ROTARIX® (Rotavirus Vaccine, Live), JYNNEOS® (Smallpox and Monkeypox Vaccine, Live), TYPHIM Vi® (Typhoid Vi Polysaccharide Vaccine), and YF-VAX® (Yellow Fever Vaccine). Exemplary COVID-19 vaccines include Pfizer-BioNTech COVID-19 vaccine, Moderna COVID-19 vaccine, Oxford/AstraZeneca COVID-19 vaccine, Russia's Sputnik V COVID-19 vaccine, and Chinese Sinopharm COVID-19 vaccine.

In some embodiments, nucleic acids are provided that express antigenic domains rather than the entire protein. These fragments may be of any length sufficient to be immunogenic or antigenic. Fragments may be at least four amino acids long, preferably 5-9 amino acids, but may be longer, such as e.g., 10, 11, 12, 13, 14, 15, 16, 17, 18, 19, 20, 30, 40, 50, 60, 70, 80, 90, 100, 500 amino acids long or more, or any length in between.

In some forms, VLPs include two species of peptide antigen for the same or different pathogen or cancer.

In preferred forms, a first antigen is designed for MHC class I presentation (i.e., MHCI epitope), and a second antigen drives MHC class II (i.e., MHCII epitope) presentation by the APC.

In some embodiments, two or more species of peptide antigens are individually displayed on the surface of the VLP at physically separate locations on the VLP. In some embodiments, two or more species of the peptide antigens are collectively displayed as a fusion protein and thus tethered to the VLP at the same physical location (i.e., a tandem linker). The two or more antigens can be separated by a linker. The linker can be a cleavable linker.

In other embodiments, two or more antigens are administered to a subject by administering a combination to two or more VLPs each displaying one antigen.

### Linkers for Antigen Presentation

In some forms, each antigenic polypeptide sequence is preceded on the peptide extension by a linker sequence that promotes processing and/or presentation of the antigen by an APC. Additionally or alternatively antigen fusion proteins having two or more antigens can include one or more of the same or different linkers separating the antigens.

A preferred linker is a protease cleavable linker. An exemplary protease cleavable linker is a linker that can be cleaved by the endosomal protease cathepsin D to promote efficient antigen processing and presentation. In some forms, the protease cleavable linker has the amino acid sequence DGSPLEF (SEQ ID NO:6).

Therefore, in some forms, the antigen is attached to the C-terminus of the VLP capsid, such as the PP7 capsid by a linker sequence and a cleavable sequence tag. An exemplary linker sequence including a cathepsin D cleavable sequence tag is the amino acid sequence.

(SEQ ID NO: 7)
GGASESGADGSPLEF.

Other peptide linkers can also be used include, but are not limited to, peptides linkers used in ADCs developments. Such linkers are known in the art and include tetra-peptides such as Gly-Phe-Leu-Gly (GFLG; SEQ ID NO:8) and Ala-Leu-Ala-Leu (ALAL; SEQ ID NO:9). These first-generation peptide linkers showed limitations in relatively slow drug release and a tendency for aggregation upon payload coupling. These issues were circumvented in with the development of new-generation dipeptide linkers such as Val-Cit and Phe-Lys linkers. These optimized small peptide linkers are cleaved by lysosomal extracts and purified human cathepsin B and they have been successfully applied in a few innovated ADCs.

Exemplary peptide antigens are described below.

### Cancer Antigens

In some forms, the peptide antigen is a cancer antigen. A cancer antigen is an antigen that is typically expressed preferentially by cancer cells (i.e., it is expressed at higher levels in cancer cells than on non-cancer cells; cancer-associated antigen) and in some instances it is expressed solely by cancer cells (cancer-specific antigen). A cancer antigen may be expressed within a cancer cell or on the surface of the cancer cell.

Exemplary cancer antigens include tumor-associated antigens (TAAs), tumor specific antigens (TSAs), tissue-specific antigens, viral tumor antigens, cellular oncogene proteins, and/or tumor-associated differentiation antigens. These antigens can serve as targets for the host immune system and elicit responses which result in tumor destruction. This immune response is mediated primarily by lymphocytes; T cells in general and class I MHC-restricted cytotoxic T lymphocytes in particular play a central role in tumor rejection. Hellstrom, et al., Adv. Cancer Res. 12:167 223 (1969); Greenberg, in Advances in Immunology, vol. 49 (Dixon, D. J., ed.), pp. 281 355, Academic Press, Inc., Orlando, FL (1991). The cloning of TAAs for cancer immunotherapy is described e.g. in Boon, et al., Annu. Rev. Immunol. 12:337 365 (1994); Brithcard, et al., J. Exp. Med. 178:489 495(1993); Cox, et al., Science 264:716 719(1994); Houghton, J. Exp. Med. 180:1 4 (1994); Pardoll, Nature 369:357 358(1994); Kawakami, et al., Proc. Natl. Acad. Sci. U.S.A. 91:3515 3519 (1994); Kawakami, et al., Proc. Natl. Acad. Sci. U.S.A. 91:6458 6462(1994).

In general, viral vaccines are believed to mediate tumor rejection by activating class I MHC-restricted T-cells, particularly cytotoxic T lymphocytes (CTLs). T-cell activation is often potentiated by providing a suitable immunomodulator, for example a T-cell co-stimulatory factor such as those of the B7 gene family. See e.g., Greenberg, in Advances in Immunology, Vol. 49 (Dixon, D. J., ed.) (1991), pp. 281 355, Academic Press, Inc., Orlando, Fla.; Fox B. A. et al. (1990) J. Biol. Response Mod. 9:499 511. The use of vaccinia viruses for anti-tumor immunotherapy has been described in Hu, S. L., Hellstrom, I., and Hellstrom K. E. (1992) in Vaccines: New Approaches to Immunological Problems (R. W. Ellis, ed) pp. 327 343, Butterworth-Heinemann, Boston. Anti-tumor responses have been elicited using recombinant pox viruses expressing TAAs such as carcinoembryonic antigen (CEA) and prostate specific antigen (PSA). (Muraro, R., et al., (1985) Cancer Res. 4S:5769 5780); (Kantor, J., et al., (1992) J. Natl. Cancer Inst. 84:1084 1091); (Robbins, P. F., et al. (1991) Cancer Res. 51:3657 3662) (Kantor, J., et al., (1992) Cancer Res. 52:6917 6925). No toxicity with these vectors was observed. However, in all cases the vaccines were injected.

Cancer antigens include, but are not limited to, melanoma TAAs such as MART-1 (Kawakami et al., J. Exp. Med. 180:347-352, 1994), MAGE-1, MAGE-3, GP-100, (Kawakami et al., Proc. Nat'l. Acad. Sci. U.S.A. 91:6458-6462, 1994), tyrosinase (Brichard et al. J. Exp. Med. 178:489, 1993), TAAs such as MUC-1, MUC-2, MUC-3, MUC-4, MUC-18, the point mutated ras oncogene and the point mutated p53 oncogenes (pancreatic cancer), PSA (prostate cancer), c-erb/B2 (breast cancer), KS 1/4 pan-carcinoma antigen (Perez and Walker, 1990, J. Immunol. 142:3662-3667; Bumal, 1988, Hybridoma 7(4):407-415), ovarian carcinoma antigen (CA125) (Yu et al., 1991, Cancer Res. 51(2):468-475), prostatic acid phosphate (Tailor et al., 1990, Nucl. Acids Res. 18(16):4928), prostate specific antigen (PSA) (Henttu and Vihko, 1989, Biochem. Biophys. Res. Comm. 160(2):903-910; Israeli et al., 1993, Cancer Res. 53:227-230), melanoma-associated antigen p97 (Estin et al., 1989, J. Natl. Cancer Instit. 81(6):445-446), melanoma antigen gp75 (Vijayasardahl et al., 1990, J. Exp. Med. 171(4):1375-1380), high molecular weight melanoma antigen (HMW-MAA) (Natali et al., 1987, Cancer 59:55-63; Mittelman et al., 1990, J. Clin. Invest. 86:2136-2144), prostate specific membrane antigen, carcinoembryonic antigen (CEA) (Foon et al., 1994, Proc. Am. Soc. Clin. Oncol. 13:294), polymorphic epithelial mucin antigen, human milk fat globule antigen, colorectal tumor-associated antigens such as TAG-72 (Yokata et al., 1992, Cancer Res. 52:3402-3408), C017-1A (Ragnhammar et al., 1993, Int. J. Cancer 53:751-758); GICA 19-9 (Herlyn et al., 1982, J. Clin. Immunol. 2:135), CTA-1 and LEA, Burkitt's lymphoma antigen-38.13, CD19 (Ghetie et al., 1994, Blood 83:1329-1336), human B-lymphoma antigen-CD20 (Reff et al., 1994, Blood 83:435-445), CD33 (Sgouros et al., 1993, J. Nucl. Med. 34:422-430), melanoma specific antigens such as ganglioside GD2 (Saleh et al., 1993, J. Immunol., 151, 3390-3398), ganglioside GD3 (Shitara et al., 1993, Cancer Immunol. Immunother. 36:373-380), ganglioside GM2 (Livingston et al., 1994, J. Clin. Oncol. 12:1036-1044), ganglioside GM3 (Hoon et al., 1993, Cancer Res. 53:5244-5250), tumor-specific transplantation type of cell-surface antigen (TSTA) such as virally induced tumor antigens including T-antigen DNA tumor viruses and Envelope antigens of RNA tumor viruses, bladder tumor oncofetal antigen (Hellstrom et al., 1985, Cancer. Res. 45:2210-2188), differentiation antigen such as human lung carcinoma antigen L6, L20 (Hellstrom et al., 1986, Cancer Res. 46:3917-3923), antigens of fibrosarcoma, human leukemia T cell antigen-Gp37 (Bhattacharya-Chatterjee et al., 1988, J. of Immuno specifically. 141:1398-1403), neoglycoprotein, sphingolipids, breast cancer antigen such as EGFR (Epidermal growth factor receptor), HER2 antigen (p185HER2), polymorphic epithelial mucin (PEM) (Hilkens et al., 1992, Trends in Bio. Chem. Sci. 17:359), malignant human lymphocyte antigen-APO-1 (Bernhard et al., 1989, Science 245:301-304), differentiation antigen (Feizi, 1985, Nature 314:53-57) such as I antigen found in fetal erythrocytes, primary endoderm, I antigen found in adult erythrocytes, preimplantation embryos, I(Ma) found in gastric adenocarcinomas, M18, M39 found in breast epithelium, SSEA-1 found in myeloid cells, VEP8, VEP9, Myl, VIM-D5, D156-22 found in colorectal cancer, TRA-1-85 (blood group H), C14 found in colonic adenocarcinoma, F3 found in lung adenocarcinoma, AH6 found in gastric cancer, Y hapten, LeY found in embryonal carcinoma cells, TL5 (blood group A), EGF receptor found in A431 cells, E1 series (blood group B) found in pancreatic cancer, FC10.2 found in embryonic carcinoma cells, gastric adenocarcinoma antigen, CO-514 (blood group Lea) found in Adenocarcinoma, NS-10 found in adenocarcinomas, CO-43 (blood group Leb), G49 found in EGF receptor of A431 cells, MH2 (blood group ALeb/Ley) found in colonic adenocarcinoma, 19.9 found in colon cancer, gastric cancer mucins, T5A7 found in myeloid cells, R24 found in melanoma, 4.2, GD3, D1.1, OFA-1, GM2, OFA-2, GD2, and M1:22:25:8 found in embryonal carcinoma cells, and SSEA-3 and SSEA-4 found in 4 to 8-cell stage embryos T cell receptor derived peptides from Cutaneous T cell Lymphoma (Edelson, 1998, The Cancer Journal 4:62), IL-1, IL-2, IL-3, IL-4, IL-5, IL-6, IL-7, IL-8, IL-9, IL-10, IL-11, IL-12, IFN-α, IFN-β, IFN-β 17 mutants, IFN-65, CD2, CD3, CD4, CD5, CD8, CD11a, CD11b, CD11c, CD16, CD18, CD21, CD28, CD32, CD34, CD35, CD40, CD44, CD45, CD54, CD56, OX40L, 4-1BBL, K2, K1, Pβ, Oα, Mα, Mβ2, Mβ1, Hepsin, Pim-1, LMP1, TAP2, LMP7, TAP1, TRP, Oβ, IAβ, IAα, IEβ, IEβ2, IEα, CYP21, C4B, CYP21P, C4A, Bf, C2, HSP, G7a/b, TNF-α, TNF-β, D, L, Qa, T1a, COL11A2, DPβ2, DPα2, DPβ1, DPα1, DNα, DMα, DMβ, LMP2, TAPi1, LMP7, DOβ, DQβ2, DQα2, DQβ3, DQβ1, DQα1, DRβ, DRα, G250, HSP-70, HLA-B, HLA-C, HLA-X, HLA-E, HLA-J, HLA-A, HLA-H, HLA-G, HLA-F, nerve growth factor, somatotropin, somatomedins, parathormone, FSH, LH, EGF, TSH, THS-releasing factor, HGH, GRHR, PDGF, IGF-I, IGF-II, TGF-β, GM-CSF, M-CSF, G-CSF1, erythropoietin, β-HCG, 4-N-acetylgalactosaminyltransferase, GM2, GD2, GD3, JADE, BAGE, GAGE, XAGE, MUC-3, MUC-4, MUC-18, ICAM-1, C-CAM, V-CAM, ELAM, NM23, EGFR, E-cadherin, N-CAM, LFA-3 (CD58), EpCAM, B7.1, DCC, UTAA, melanoma antigen p75, K19, HKer 8, pMel 17, TP10, tyrosinase related proteins 1 and 2, p97, p53, RB, APC, DCC, NF-1, NF-2, WT-1, MEN-I, MEN-II, BRCA1, VHL, FCC and MCC, ras, myc, neu, raf, erb, src, fms, jun, trk, ret, gsp, hst, bcl and abl, HRF, MIRL, CR1, CR2, CR3, CR4, C3a/C4a receptor, C5a receptor, Epstein-Barr Virus antigens (EBNA), BZLF-1, BXLF-1, and Nuclear Matrix Proteins, modified TAAs or TSAs, splice variants of TAAs or TSAs, functional epitopes, epitope agonists, and degenerate nucleic acid variations thereof.

### Patient-Specific Cancer Antigens

In some forms, the peptide cancer antigen is a neoantigen or a patient-specific antigen. Recent technological improvements have made it possible to identify the immune response to patient-specific neoantigens that arise as a consequence of tumor-specific mutations, and emerging data indicate that recognition of such neoantigens is a major factor in the activity of clinical immunotherapies (Schumacher and Schreidber, Science, 348(6230):69-74 (2015). Neoantigen load provides an avenue to selectively enhance T cell reactivity against this class of antigens.

Traditionally, cancer vaccines have targeted tumor-associated antigens (TAAs) which can be expressed not only on tumor cells but in the normal tissues (Ito, et al., Cancer Neoantigens: A Promising Source of Immunogens for Cancer Immunotherapy. J Clin Cell Immunol, 6:322 (2015) doi:10.4172/2155-9899.1000322). TAAs include cancer-testis antigens and differentiation antigens, and even though self-antigens have the benefit of being useful for diverse patients, expanded T cells with the high-affinity TCR (T-cell receptor) needed to overcome the central and peripheral tolerance of the host, which would impair anti-tumor T-cell activities and increase risks of autoimmune reactions.

In some forms, the antigen is recognized as “non-self” by the host immune system, and preferably can bypass central tolerance in the thymus. Examples include pathogen-associated antigens, mutated growth factor receptor, mutated K-ras, or idiotype-derived antigens. Somatic mutations in tumor genes, which usually accumulate tens to hundreds of fold during neoplastic transformation, could occur in protein-coding regions. Whether missense or frameshift, every mutation has the potential to generate tumor-specific antigens. These mutant antigens can be referred to as “cancer neoantigens” Ito, et al., Cancer Neoantigens: A Promising Source of Immunogens for Cancer Immunotherapy. J Clin Cell Immunol, 6:322 (2015) doi:10.4172/2155-9899.1000322. Neoantigen-based cancer vaccines have the potential to induce more robust and specific anti-tumor T-cell responses compared with conventional shared-antigen-targeted vaccines. Recent developments in genomics and bioinformatics, including massively parallel sequencing (MPS) and epitope prediction algorithms, have provided a major breakthrough in identifying and selecting neoantigens.

### Viral Antigens

In some forms, the peptide antigen is a viral antigen. A viral antigen can be isolated from any virus including, but not limited to, a virus from any of the following viral families: Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Caulimovirus, Circoviridae, Closterovirus, Comoviridae, Coronaviridae (e.g., Coronavirus, such as severe acute respiratory syndrome (SARS) virus), Corticoviridae, Cystoviridae, Deltavirus, Dianthovirus, Enamovirus, Filoviridae (e.g., Marburg virus and Ebola virus (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hepadnaviridae, Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae (e.g., Influenzavirus A and B and C), Papovaviridae, Paramyxoviridae (e.g., measles, mumps, and human respiratory syncytial virus), Parvoviridae, Picornaviridae (e.g., poliovirus, rhinovirus, hepatovirus, and aphthovirus), Poxviridae (e.g., vaccinia and smallpox virus), Reoviridae (e.g., rotavirus), Retroviridae (e.g., lentivirus, such as human immunodeficiency virus (HIV) 1 and HIV 2), Rhabdoviridae (for example, rabies virus, measles virus, respiratory syncytial virus, etc.), Togaviridae (for example, rubella virus, dengue virus, etc.), and Totiviridae. Suitable viral antigens also include all or part of Dengue protein M, Dengue protein E, Dengue D1NS1, Dengue D1NS2, and Dengue D1NS3.

Viral antigens may be derived from a particular strain such as a papilloma virus, a herpes virus, e.g., herpes simplex 1 and 2; a hepatitis virus, for example, hepatitis A virus (HAV), hepatitis B virus (HBV), hepatitis C virus (HCV), the delta hepatitis D virus (HDV), hepatitis E virus (HEV) and hepatitis G virus (HGV), the tick-borne encephalitis viruses; parainfluenza, varicella-zoster, cytomeglavirus, Epstein-Barr, rotavirus, rhinovirus, adenovirus, coxsackieviruses, equine encephalitis, Japanese encephalitis, yellow fever, Rift Valley fever, and lymphocytic choriomeningitis.

### Bacterial Antigens

In some forms, the peptide antigen is a bacterial antigen. Bacterial antigens can originate from any bacteria including, but not limited to, Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Hyphomicrobium, Legionella, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, and Yersinia.

### Parasite Antigens

In some forms, the peptide antigen is a protozoan antigen, or an antigen derived from another parasite. Parasite antigens can be obtained from parasites such as, but not limited to, an antigen derived from Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis and Schistosoma mansoni. These include Sporozoan antigens, Plasmodian antigens, such as all or part of a Circumsporozoite protein, a Sporozoite surface protein, a liver stage antigen, an apical membrane associated protein, or a Merozoite surface protein.

Allergens and environmental antigens In some forms, the peptide antigen is an allergen or environmental antigen. The antigen can be an allergen or environmental antigen, such as, but not limited to, an antigen derived from naturally occurring allergens such as pollen allergens (tree-, herb, weed-, and grass pollen allergens), insect allergens (inhalant, saliva and venom allergens), animal hair and dandruff allergens, and food allergens. Important pollen allergens from trees, grasses and herbs originate from the taxonomic orders of Fagales, Oleales, Pinales and platanaceae including i.a. birch (Betula), alder (Alnus), hazel (Corylus), hornbeam (Carpinus) and olive (Olea), cedar (Cryptomeria and Juniperus), Plane tree (Platanus), the order of Poales including e.g., grasses of the genera Lolium, Phleum, Poa, Cynodon, Dactylis, Holcus, Phalaris, Secale, and Sorghum, the orders of Asterales and Urticales including i.a. herbs of the genera Ambrosia, Artemisia, and Parietaria. Other allergen antigens that may be used include allergens from house dust mites of the genus Dermatophagoides and Euroglyphus, storage mite e.g Lepidoglyphys, Glycyphagus and Tyrophagus, those from cockroaches, midges and fleas e.g. Blatella, Periplaneta, Chironomus and Ctenocepphalides, those from mammals such as cat, dog and horse, birds, venom allergens including such originating from stinging or biting insects such as those from the taxonomic order of Hymenoptera including bees (superfamily Apidae), wasps (superfamily Vespidea), and ants (superfamily Formicoidae). Still other allergen antigens that may be used include inhalation allergens from fungi such as from the genera Alternaria and Cladosporium.

### Tolerogenic Antigens

In some forms, the peptide antigen is a tolerogenic antigen. Exemplary tolerogenic antigens are known in the art. See, for example, U.S. Published Application No. 2014/0356384.

In some cases, the tolerogenic antigen is derived from a therapeutic agent protein to which tolerance is desired. Examples are protein drugs in their wild type, e.g., human factor VIII or factor IX, to which patients did not establish central tolerance because they were deficient in those proteins; or nonhuman protein drugs, used in a human. Other examples are protein drugs that are glycosylated in nonhuman forms due to production, or engineered protein drugs, e.g., having non-native sequences that can provoke an unwanted immune response. Examples of tolerogenic antigens that are engineered therapeutic proteins not naturally found in humans including human proteins with engineered mutations, e.g., mutations to improve pharmacological characteristics. Examples of tolerogenic antigens that have nonhuman glycosylation include proteins produced in yeast or insect cells.

Tolerogenic antigens can be from proteins that are administered to humans that are deficient in the protein. Deficient means that the patient receiving the protein does not naturally produce enough of the protein. Moreover, the proteins may be proteins for which a patient is genetically deficient. Such proteins include, for example, antithrombin-III, protein C, factor VIII, factor IX, growth hormone, somatotropin, insulin, pramlintide acetate, mecasermin (IGF-1), β-gluco cerebrosidase, alglucosidase-.alpha., laronidase (α-L-iduronidase), idursuphase (iduronate-2-sulphatase), galsulphase, agalsidase-β (α-galactosidase), α-1 proteinase inhibitor, and albumin.

The tolerogenic antigen can be from therapeutic antibodies and antibody-like molecules, including antibody fragments and fusion proteins with antibodies and antibody fragments. These include nonhuman (such as mouse) antibodies, chimeric antibodies, and humanized antibodies. Immune responses to even humanized antibodies have been observed in humans (Getts D R, Getts M T, McCarthy D P, Chastain E M L, & Miller S D (2010), mAbs, 2(6):682-694).

The tolerogenic antigen can be from proteins that are nonhuman. Examples of such proteins include adenosine deaminase, pancreatic lipase, pancreatic amylase, lactase, botulinum toxin type A, botulinum toxin type B, collagenase, hyaluronidase, papain, L-Asparaginase, rasburicase, lepirudin, streptokinase, anistreplase (anisoylated plasminogen streptokinase activator complex), antithymocyte globulin, crotalidae polyvalent immune Fab, digoxin immune serum Fab, L-arginase, and L-methionase.

Tolerogenic antigens include those from human allograft transplantation antigens. Examples of these antigens are the subunits of the various MHC class I and MHC class II haplotype proteins, and single-amino-acid polymorphisms on minor blood group antigens including RhCE, Kell, Kidd, Duffy and Ss.

The tolerogenic antigen can be a self-antigen against which a patient has developed an autoimmune response or may develop an autoimmune response. Examples are proinsulin (diabetes), collagens (rheumatoid arthritis), myelin basic protein (multiple sclerosis). For instance, Type 1 diabetes mellitus (T1D) is an autoimmune disease whereby T cells that recognize islet proteins have broken free of immune regulation and signal the immune system to destroy pancreatic tissue. Numerous protein antigens that are targets of such diabetogenic T cells have been discovered, including insulin, GAD65, chromogranin-A, among others. In the treatment or prevention of T1D, it would be useful to induce antigen-specific immune tolerance towards defined diabetogenic antigens to functionally inactivate or delete the diabetogenic T cell clones.

Tolerance and/or delay of onset or progression of autoimmune diseases may be achieved for various of the many proteins that are human autoimmune proteins, a term referring to various autoimmune diseases wherein the protein or proteins causing the disease are known or can be established by routine testing. In some embodiments, a patient is tested to identify an autoimmune protein and an antigen is created for use in a molecular fusion to create immunotolerance to the protein.

Embodiments can include an antigen, or choosing an antigen from or derived from, one or more of the following proteins. In type 1 diabetes mellitus, several main antigens have been identified: insulin, proinsulin, preproinsulin, glutamic acid decarboxylase-65 (GAD-65), GAD-67, insulinoma-associated protein 2 (IA-2), and insulinoma-associated protein 2.beta. (IA-213); other antigens include ICA69, ICA12 (SOX-13), carboxypeptidase H, Imogen 38, GLIMA 38, chromogranin-A, FISP-60, caboxypeptidase E, peripherin, glucose transporter 2, hepatocarcinoma-intestine-pancreas/pancreatic associated protein, S100β, glial fibrillary acidic protein, regenerating gene II, pancreatic duodenal homeobox 1, dystrophia myotonica kinase, islet-specific glucose-6-phosphatase catalytic subunit-related protein, and SST G-protein coupled receptors 1-5. In autoimmune diseases of the thyroid, including Hashimoto's thyroiditis and Graves' disease, main antigens include thyroglobulin (TG), thyroid peroxidase (TPO) and thyrotropin receptor (TSHR); other antigens include sodium iodine symporter (NIS) and megalin. In thyroid-associated ophthalmopathy and dermopathy, in addition to thyroid autoantigens including TSHR, an antigen is insulin-like growth factor 1 receptor. In hypoparathyroidism, a main antigen is calcium sensitive receptor. In Addison's disease, main antigens include 21-hydroxylase, 17α-hydroxylase, and P450 side chain cleavage enzyme (P450scc); other antigens include ACTH receptor, P450c21 and P450c17. In premature ovarian failure, main antigens include FSH receptor and .alpha.-enolase. In autoimmune hypophysitis, or pituitary autoimmune disease, main antigens include pituitary gland-specific protein factor (PGSF) 1a and 2; another antigen is type 2 iodothyronine deiodinase. In multiple sclerosis, main antigens include myelin basic protein, myelin oligodendrocyte glycoprotein and proteolipid protein. In rheumatoid arthritis, a main antigen is collagen II. In immunogastritis, a main antigen is H+, K+-ATPase. In pernicious angemis, a main antigen is intrinsic factor. In celiac disease, main antigens are tissue transglutaminase and gliadin. In vitiligo, a main antigen is tyrosinase, and tyrosinase related protein 1 and 2. In myasthenia gravis, a main antigen is acetylcholine receptor. In pemphigus vulgaris and variants, main antigens are desmoglein 3, 1 and 4; other antigens include pemphaxin, desmocollins, plakoglobin, perplakin, desmoplakins, and acetylcholine receptor. In bullous pemphigoid, main antigens include BP180 and BP230; other antigens include plectin and laminin 5. In dermatitis herpetiformis Duhring, main antigens include endomysium and tissue transglutaminase. In epidermolysis bullosa acquisita, a main antigen is collagen VII. In systemic sclerosis, main antigens include matrix metalloproteinase 1 and 3, the collagen-specific molecular chaperone heat-shock protein 47, fibrillin-1, and PDGF receptor; other antigens include Scl-70, U1 RNP, Th/To, Ku, Jol, NAG-2, centromere proteins, topoisomerase I, nucleolar proteins, RNA polymerase I, II and III, PM-Slc, fibrillarin, and B23. In mixed connective tissue disease, a main antigen is U1snRNP. In Sjogren's syndrome, the main antigens are nuclear antigens SS-A and SS-B; other antigens include fodrin, poly(ADP-ribose) polymerase and topoisomerase. In systemic lupus erythematosus, main antigens include nuclear proteins including SS-A, high mobility group box 1 (HMGB1), nucleosomes, histone proteins and double-stranded DNA. In Goodpasture's syndrome, main antigens include glomerular basement membrane proteins including collagen IV. In rheumatic heart disease, a main antigen is cardiac myosin. Other autoantigens revealed in autoimmune polyglandular syndrome type 1 include aromatic L-amino acid decarboxylase, histidine decarboxylase, cysteine sulfinic acid decarboxylase, tryptophan hydroxylase, tyrosine hydroxylase, phenylalanine hydroxylase, hepatic P450 cytochromes P4501A2 and 2A6, SOX-9, SOX-10, calcium-sensing receptor protein, and the type 1 interferons interferon alpha, beta and omega.

In some cases, the tolerogenic antigen is a foreign antigen against which a patient has developed an unwanted immune response. Examples are food antigens. Some embodiments include testing a patient to identify foreign antigen and creating a molecular fusion that comprises the antigen and treating the patient to develop immunotolerance to the antigen or food. Examples of such foods and/or antigens are provided. Examples are from peanut: conarachin (Ara h 1), allergen II (Ara h 2), arachis agglutinin, conglutin (Ara h 6); from apple: 31 kda major allergen/disease resistance protein homolog (Mal d 2), lipid transfer protein precursor (Mal d 3), major allergen Mal d 1.03D (Mal d 1); from milk: .alpha.-lactalbumin (ALA), lactotransferrin; from kiwi: actinidin (Act c 1, Act d 1), phytocystatin, thaumatin-like protein (Act d 2), kiwellin (Act d 5); from mustard: 2S albumin (Sin a 1), 11 S globulin (Sin a 2), lipid transfer protein (Sin a 3), profilin (Sin a 4); from celery: profilin (Api g 4), high molecular weight glycoprotein (Api g 5); from shrimp: Pen a 1 allergen (Pen a 1), allergen Pen m 2 (Pen in 2), tropomyosin fast isoform; from wheat and/or other cereals: high molecular weight glutenin, low molecular weight glutenin, alpha- and gamma-gliadin, hordein, secalin, avenin; from strawberry: major strawberry allergy Fra a 1-E (Fra a 1), from banana: profilin (Mus xp 1).

Many protein drugs that are used in human and veterinary medicine induce immune responses, which create risks for the patient and limits the efficacy of the drug. This can occur with human proteins that have been engineered, with human proteins used in patients with congenital deficiencies in production of that protein, and with nonhuman proteins. It would be advantageous to tolerize a recipient to these protein drugs prior to initial administration, and it would be advantageous to tolerize a recipient to these protein drugs after initial administration and development of immune response. In patients with autoimmunity, the self-antigen(s) to which autoimmunity is developed are known. In these cases, it would be advantageous to tolerize subjects at risk prior to development of autoimmunity, and it would be advantageous to tolerize subjects at the time of or after development of biomolecular indicators of incipient autoimmunity. For example, in Type 1 diabetes mellitus, immunological indicators of autoimmunity are present before broad destruction of beta cells in the pancreas and onset of clinical disease involved in glucose homeostasis.

e. Exemplary Targeting Ligands

The VLPs are modified by attachment of one or more molecules that selectively target APCs to the surface of the VLPs.

Exemplary APC targeting molecules include ligands for lectins expressed at the surface of APCs. In some forms, the VLPs are modified to present one or more molecules for selective targeting and/or binding of the compositions to dendritic cells in vivo. Exemplary DC targeting ligands include molecules that target DC-SIGN, such as carbohydrates.

For example, the attachment of molecules to the surface of VLPs is carried out by conjugation of an azide-terminated succinimidyl ester linker to acetylate amino groups, resulting in a random and even distribution of azides on the particle surface. These intermediate particles are then decorated with one or more forms of alkyne-functionalized ligands.

In some forms, higher densities of ligand presentation increases binding avidity to DC surface receptors, such as the tetrameric DC-SIGN receptor and promote the receptor clustering needed for antigen uptake and intracellular signaling. Therefore, in some forms, VLPs include high density clustering of molecules that target and/or bind to DCs.

In some forms, the VLPs are modified to display one or more molecules for selective targeting and/or binding of the compositions to Dendritic Cells (DC) in vivo.

Attractive targets for DC-targeting are cell surface lectins. Lectins are carbohydrate-binding proteins that mediate important cell-cell interactions including fertilization, pathogen invasion, and immune system activation or attenuation. DCs express lectins to recognize and bind unique glycan displays on the surface of pathogens, facilitating highly efficient internalization of antigens at very low (nanomolar) concentrations. Additionally, lectins use glycan-mediated pathogen recognition to regulate cytokine secretion/gene expression for directed T cell activation. Therefore, in some forms, the VLPs are modified by attachment of ligands that bind lectins expressed at the surface of DCs.

In some forms, the VLPs are modified to include molecules that target and/or bind the VLPs to one or more C-type lectin receptors (CLRs) on DCs in vivo.

CLRs at the surface of DCs are important pattern recognition receptors that play an important role in the induction of adaptive immunity to pathogens, especially viruses. CLRs that serve as endocytic receptors enhance antigen presentation on MHC molecules.

CLRs facilitate uptake of carbohydrate antigens for antigen presentation, modulating the immune response in infection, homeostasis, autoimmunity, allergy, and cancer.

Exemplary CLRs include DC-SIGN, Dectin and MGL. Most lectins, including DC-SIGN, are oligomers that prefer multivalent protein-carbohydrate interactions and receptor clustering for efficient signal transduction.

i. DC-SIGN Ligands

In preferred forms, the VLPs are modified to include molecules that target and/or bind the VLPs to dendritic cell-specific intercellular adhesion molecule-3-grabbing nonintegrin (DC SIGN; CD209) at the surface of DC cells.

DC-SIGN is a tetrameric CLR that binds mannose- and fucose-to serve as a receptor for viruses, bacteria, yeast, and parasites. Importantly, DC-SIGN can induce the immunostimulatory cellular TH1/CTL-type immune response that is desired for an anti-tumor response. DC-SIGN expressed at the surface of DCs it is a high-affinity receptor for ICAM2 and ICAM3 by binding to mannose-like carbohydrates.

DC-SIGN can act as a DC rolling receptor that mediates transendothelial migration of DC presursors from blood to tissues by binding endothelial ICAM2, and regulates DC-induced T-cell proliferation by binding to ICAM3 on T-cells in the immunological synapse formed between DC and T-cells.

DC-SIGN recognizes and internalizes antigens bearing mannosylated glycans. Therefore, in preferred forms, VLPs are modified by attachment of mannosylated glycans to the outer surface of the VLPs.

Exemplary mannose-bearing ligands for conjugation onto the surface of VLPs include phenyl mannoside, as depicted in Formula I.

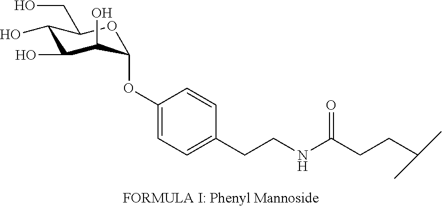

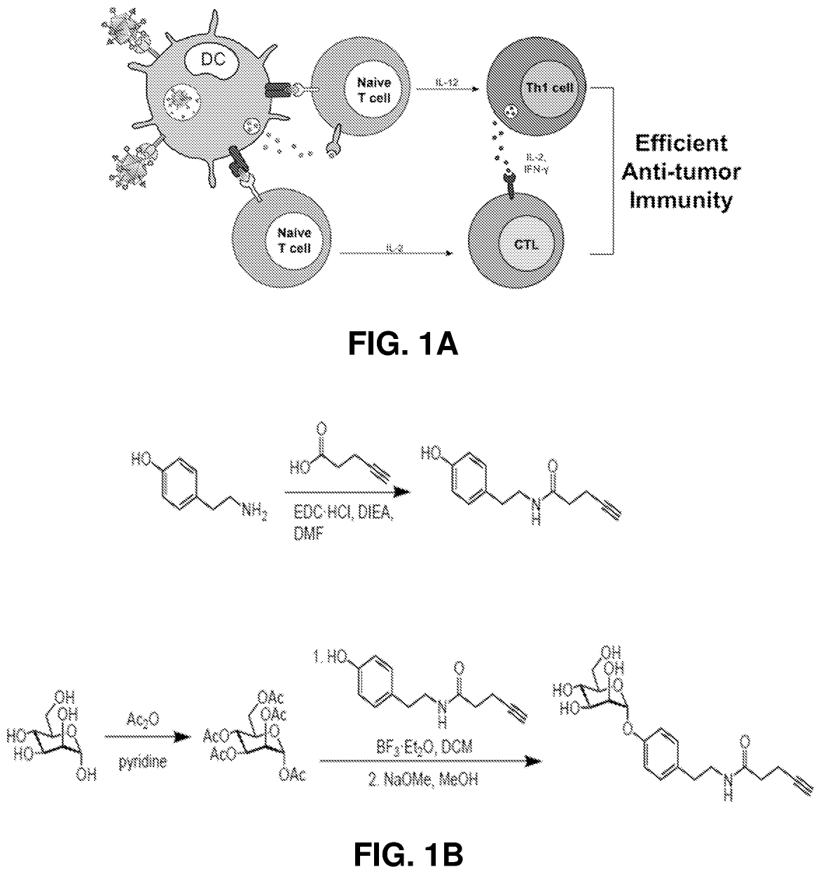

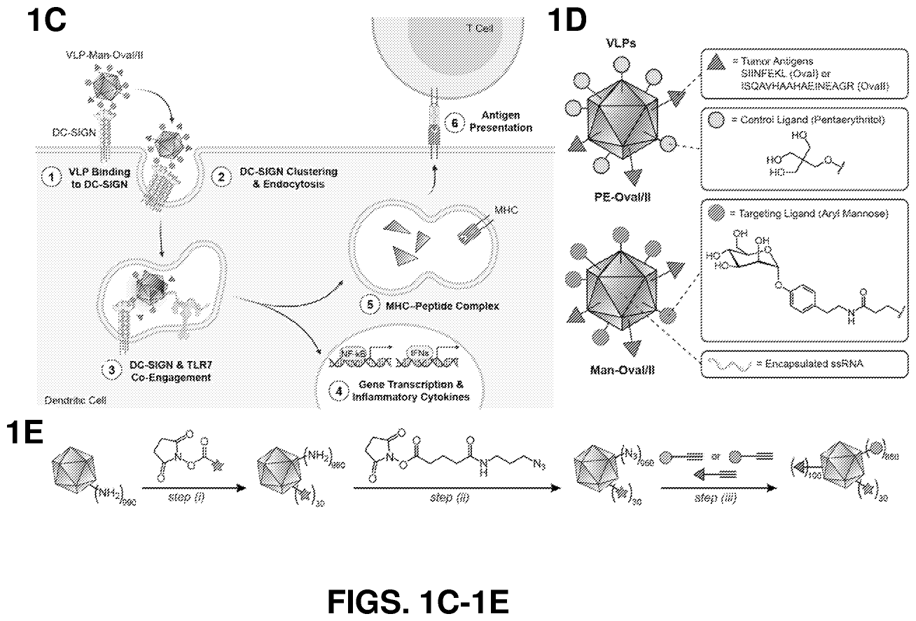

Schemes for the production of phenyl-manoside ‘antigens’ for conjugation to the surface of VLPs include scheme I (below), as well as the scheme set forth in FIG. 1B.

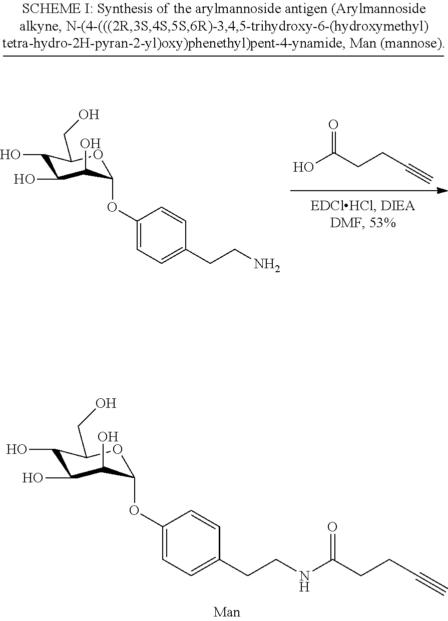

In some forms, the arylmannoside antigen is prepared by addition of a hexose (e.g, fucose) or aldohexose (e.g., mannose) moiety to a pre-formed aryl-bearing core structure, for example, as depicted in FIG. 1B. Thus, the scheme of FIG. 1B can be used to prepare aryl-bearing cores displaying alternative monosaccharides or oligosaccharides or glycans, by substituting the mannopyranose with the alternative monosaccharide(s) or oligosaccharide(s) or glycan(s). In a particular embodiment, the alternative monosaccharide(s) or oligosaccharide(s) or glycan(s) is or includes fucose.

In some forms, VLPs are treated with a high concentration of an azide-terminated succinimidyl ester linker to acetylate amino groups, resulting in a random and even distribution of azides on the particle surface. These intermediate particles are then decorated with one or more forms of alkyne-functionalized mannose ligands. Because of the presence of a hydrophobic pocket near the carbohydrate-binding site, an α-O-aryl mannoside derivative (Man, Formula I) is used as a mannose moiety.

In other forms, the mannose moiety is a trimannoside (Man3, Formula II), which lacks the aryl substituent.

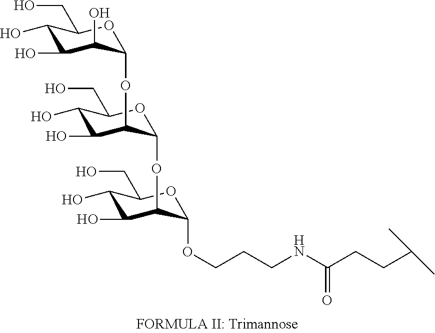

Results indicate that trimannose does not show the pH independent binding of the aryl conjugate of Formula I, and thus was not as good at invoking the immune response.

An exemplary control moiety for binding to VLPs is a pentaerythritol-derived triol (PE), which can mimic the polyhydroxylated nature of the carbohydrate but not its receptor-relevant structure.

ii. Other CLR Ligands

In other forms, the VLPs are modified by attachment of a ligand for a CLR that is not DC-SIGN. For example, in some forms, VLPs are modified by surface attachment of one or more ligands for DC-SIGN, as well as for attachment of a CLR that is not DC-SIGN. Exemplary CLRs in addition to DC-SIGN include Dectin and macrophage galactose type C-type lectin (MGL).

In some forms, VLPs are modified to include one or more Dectin ligands attached to the surface of the VLP. Dectin-1 is a mainly myeloid-cell-expressed NK-cell-receptor-like C-type lectin that functions as a transmembrane pattern-recognition receptor through its ability to bind β3-glucan carbohydrates. Dectin-1 also recognizes an unidentified endogenous ligand on T cells, possibly acting as a co-stimulatory molecule.

An exemplary Dectin ligand is set forth in Formula III.

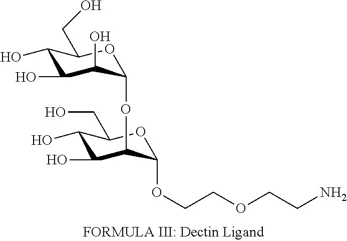

Diaminobutane amine polypropylenimine tetramine (DAB Am 4) is a polymer with a 1,4-diaminobutane core (4-carbon core) with 4 surface primary amino groups.

In some forms, VLPs are modified to include one or more ligands for the macrophage galactose type C-type lectin (MGL) attached to the surface of the VLP. MGL functions in the immune response against self-antigens, pathogens, and tumor associated antigens (TAA). MGL is a CLR exclusively expressed by dendritic cells (DCs) and activated macrophages (MOs), able to recognize terminal GalNAc residues, including the sialylated and nonsialylated Tn antigens. Modifying Tn-density, the length, and steric structure of the Tn-antigens can result in generating immunogens that can efficiently bind to MGL, strongly activate DCs, mimic the effects of a danger signal, and achieve an efficient presentation in HLA classes I and II compartments.

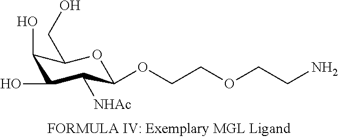

### 3. Encapsidation by VLPs

In some forms, the VLPs encapsidate one or more active agents within the VLP. Encapsidated agents can be nucleic acids, peptides, proteins, lipids, small molecules, carbohydrates or combinations thereof.

In preferred forms, the VLPs encapsidate immunostimulatory molecules that act as a self-adjuvanting agent. In some forms, the VLPs encapsidate nucleic acids that are immunostimulatory. Exemplary immunostimulatory nucleic acids include TLR agonists. Exemplary immunostimulatory nucleic acid TLR agonists include microbial nucleic acids, such as bacterial nucleic acids and viral nucleic acids. Exemplary bacterial nucleic acids for encaspidation include bacterial RNA, such as bacterial RNA encapsidated by the wildtype Leviviridae virus.

a. Immunostimulatory Agents

In some forms, the active agents encapsulated within the VLPs include one or more nucleic acids. Representative examples of nucleic acids include Toll like receptor (TLR) ligands, such as nucleic acids.

In certain forms, the VLPs encapsulate one or more immunomodulatory molecules, or nucleic acids, which are generated to direct the immune response specifically toward a T-helper cell 1 (Th1; cellular) or T-helper cell 2 (Th2; humoral) polarization for a delivered antigen.

This may be to enhance functional immunity against the disease associated with the antigen by promoting the Th pathway correlated with protection, or to achieve tolerance by directing the immune response away from the Th pathway correlated with an inappropriate immune response to the antigen.

Therefore, in some forms, VLPs include molecules or proteins that drive cellular immunity against an antigen, e.g., in order to decrease the humoral response and thus treat an allergy. This principle may be applied to tolerize against self-antigens responsible for autoimmune diseases, for example, by driving a Th2 response to curtail the Th1-associated pathology of multiple sclerosis or rheumatoid arthritis. Exemplary immunomodulatory molecules include synthetic receptor ligand or protein, cytokines or other signaling molecules.

In some instances, class-switching of B cells toward non-inflammatory antibody isotypes (i.e., immunoglobulin A (IgA) and immunoglobulin G (IgG4)), and away from the immunoglobulin E (IgE) isotype associated with allergic responses, may be desired. Therefore, in some forms, VLPs enclose or encode molecules that can drive class-switching of B cells toward non-inflammatory antibody isotypes. Exemplary immune-modulatory molecules include Interleukins, such as IL-10, and TLR agonists, such as bacterial RNA.

### TLR Agonists

In some forms, the VLPs encapsidate one or more immunostimulatory nucleic acids, such as Toll like receptor (TLR) ligands.

In some forms, the immunostimulatory oligonucleotide is a ligand for a Pattern Recognition Receptor (PRR). Examples of PRRs include the Toll-like family of signaling molecules that play a role in the initiation of innate immune responses and also influence the later and more antigen specific adaptive immune responses. Therefore, the oligonucleotide can serve as a ligand for a Toll-like family signaling molecule, such as Toll-Like Receptor 9 (TLR9).

Unmethylated CpG sites can be detected by TLR9 on plasmacytoid dendritic cells and B cells in humans (Zaida, et al., Infection and Immunity, 76(5):2123-2129, (2008)). Therefore, the sequence of the oligonucleotide can include one or more unmethylated cytosine-guanine (CG or CpG, used interchangeably) dinucleotide motifs. The ‘p’ refers to the phosphodiester backbone of DNA, as discussed in more detail below, some oligonucleotides including CG can have a modified backbone, for example a phosphorothioate (PS) backbone.

In some forms, an immunostimulatory oligonucleotide can contain more than one CG dinucleotide, arranged either contiguously or separated by intervening nucleotide(s). The CpG motif(s) can be in the interior of the oligonucleotide sequence. Numerous nucleotide sequences stimulate TLR9 with variations in the number and location of CG dinucleotide(s), as well as the precise base sequences flanking the CG dimers.

Typically, CG ODNs are classified based on their sequence, secondary structures, and effect on human peripheral blood mononuclear cells (PBMCs). The five classes are Class A (Type D), Class B (Type K), Class C, Class P, and Class S (Vollmer, J & Krieg, A M, Advanced drug delivery reviews 61(3): 195-204 (2009), incorporated herein by reference). CG ODNs can stimulate the production of Type I interferons (e.g., IFNα) and induce the maturation of dendritic cells (DCs). Some classes of ODNs are also strong activators of natural killer (NK) cells through indirect cytokine signaling. Some classes are strong stimulators of human B cell and monocyte maturation (Weiner, G L, PNAS USA 94(20): 10833-7 (1997); Dalpke, A H, Immunology 106(1): 102-12 (2002); Hartmann, G, J of Immun. 164(3):1617-2 (2000), each of which is incorporated herein by reference).

Other PRR Toll-like receptors include TLR3, and TLR7 which may recognize double-stranded RNA, single-stranded and short double-stranded RNAs, respectively, and retinoic acid-inducible gene I (RIG-I)-like receptors, namely RIG-I and melanoma differentiation-associated gene 5 (MDA5), which are best known as RNA-sensing receptors in the cytosol. Therefore, in some forms, the oligonucleotide contains a functional ligand for TLR3, TLR7, or RIG-I-like receptors, or combinations thereof.

In some forms, the TLR agonist is RIG-I (retinoic-acid-inducible protein 1, also known as Ddx58). RIG-I and MDA-5 (melanoma-differentiation-associated gene 5, also known as Ifih1 or Helicard) are cytoplasmic RNA helicases that belong to the RIG-I-like receptors (RLRs) family and are critical for host antiviral responses.

RIG-I and MDA-5 sense double-stranded RNA (dsRNA), a replication intermediate for RNA viruses, and signal through the mitochondrial antiviral signaling protein MAVS (also known as IPS-1, VISA or Cardif), leading to production of type-I interferons (IFN-α and IFN-β).

RIG-I detects viral RNA that exhibit an uncapped 5′-di/triphosphate end and a short blunt-ended double stranded portion, two essential features facilitating discrimination from self-RNAs. The features of MDA-5 physiological ligands have not been fully characterized yet. However, it is admitted that RIG-I and MDA-5 exhibit a different dependency for the length of dsRNAs: RIG-I selectively binds short dsRNA while MDA-5 selectively binds long dsRNA. Consistent with this, RIG-I and MDA-5 bind Poly(I:C), a synthetic dsRNA analog, with different length predilection. Under some circumstances, RIG-I can also sense dsDNA indirectly. Viral dsDNA can be transcribed by the RNA polymerase III into dsRNA with a 5′-triphosphate moiety. Poly(dA:dT), a synthetic analog of B-form DNA, thus constitutes another RIG-I ligand.

Exemplary RIG-I ligands include, but are not limited to, 5′ppp-dsRNA, a specific agonist of RIG-I; 3p-hpRNA, a specific agonist of RIG-I; Poly(I:C)/LyoVec complexes that are recognized by RIG-I and/or MDA-5 depending of the size of poly(I:C); Poly(dA:dT)/LyoVec complexes that are indirectly recognized by RIG-I.

In some embodiments, the oligonucleotide contains a functional ligand for TLR3, TLR7, TLR8, TLR9, or RIG-I-like receptors, or combinations thereof. Examples of immunostimulatory oligonucleotides, and methods of making them are known in the art and commercially available, see for example, Bodera, P. Recent Pat Inflamm Allergy Drug Discov. 5(1):87-93 (2011), incorporated herein by reference.

In some forms, the oligonucleotide includes two or more immunostimulatory sequences.

### Microbial Nucleic Acids

In some forms, the VLPs encapsidate microbial nucleic acids, such as microbial RNA. Microbial RNA is an important stimulator of innate immune responses. Differences in posttranscriptional RNA modification profiles enable the immune system to discriminate between self and non-self nucleic acids.

The innate immune system serves an important function in the early sensing and clearance of pathogens which is achieved by the recognition of conserved pathogen-associated molecular patterns by different pattern recognition receptors. Nucleic acids of both viral and bacterial origin constitute an important group of pathogen-associated molecular patterns that trigger a variety of cytosolic (RIG-I, MDA5, NLRP3, AIM2, cGAS, IFI16) and endosomal receptors (TLR3, 7, 8, 9 and in the murine system TLR13). Due to the pronounced chemical and structural similarities of host and microbial nucleic acids, discrimination between self and non-self is a fundamental but challenging task. Three main principles for distinction of self/non-self nucleic acids have been deciphered, including subcellular compartmentalization, sequence composition and specific nucleotide modifications. In the case of RNA, more than 100 different modifications have been identified that are all introduced post-transcriptionally. Importantly, the extent and kind of nucleotide modifications incorporated varies significantly depending on the RNA species and its evolutionary origin. Eukaryotic RNA is more abundantly modified than prokaryotic RNA, and tRNAs contain most modifications.

Therefore, in some forms, the VLPs encapsidate microbial RNAs, such as bacterial or viral RNAs. In preferred forms, the VLP encapsidates bacterial RNA. It may be that the encapsidated RNAs are relocated into the cytosol of a host APC upon uptake of the VLPs and subsequent lysosomal degradation. It may also be that the microbial RNA is processed and/or recognized by PPRs to stimulate innate immune responses by the APC, thereby providing enhanced immunostimulation in a host subject.

Exemplary microbial nucleic acids include bacterial RNA, such as E. coli RNA and viral nucleic acids, such Leviviridae-derived nucleic acids and MS-2-derived nucleic acids.

### STING Agonists

In some forms, the VLPs encapsidate one or more agents that act as STING agonists.

STING agonists are small molecule analogue of cyclic GMP-AMP (cGAMP) that acts as an agonist of the stimulator of interferon genes protein (STING; transmembrane protein 173; TMEM173) with potential immunoactivating and antineoplastic activities.

In some forms, the VLPs encapsidate functional nucleic acids that encode a cyclic dinucleotide STING agonist. Cyclic dinucleotides bind directly to the STING adaptor protein, resulting in production of IFN-β (Zhang, et al., Mol Cell., 51(2):226-35 (2013). Doi: 10.1016/j.molcel.2013.05.022.). Exemplary canonical and noncanonical dinucleotides that can be encapsidated by the VLPs include, but are not limited to, 2′3′-cGAMP, 2′3′-cGAMP, 3′3′-cGAMP, c-di-AMP, c-di-GMP, cAIMP (CL592), cAIMP Difluor (CL614), cAIM(PS)2 Difluor (Rp/Sp) (CL656), 2′2′-cGAMP, 2′3′-cGAM(PS)2 (Rp/Sp), 3′3′-cGAMP Fluorinated, c-di-AMP Fluorinated, 2′3′-c-di-AMP, 2′3′-c-di-AM(PS)2 (Rp,Rp), 2′3′-c-di-AM(PS)2 (Rp,Rp), c-di-GMP Fluorinated, 2′3′-c-di-GMP, c-di-IMP, DMXAA.

b. Other Active Agents

In some forms, therapeutic, prophylactic and diagnostic agents encapsulated within the VLPs.

Thus, the VLPs can encapsidate one or more one or more other active agents in addition or alternative to one or more immunostimulatory agents, such as, but not limited to, those mentioned above.

In an exemplary form, the VLPs enclose/contain an active agent selected from a functional nucleic acid, a polypeptide or protein, a small molecule, a lipid, carbohydrate, or combinations thereof.

The convenient packaging of enzymes bearing a positively charge, such as a Rev oligopeptide, when co-expressed with a viral capsid protein and a bridging non-translated RNA are known in the art. Therefore, in some forms, VLPs are designed to encapsidate one or more protein or peptides that are active agents.

A non-limiting list of active agents that can be encapsulated within, or associated with the surface of the VLPs includes anti-infectives, immunomodifying agents, hormones, antioxidants, steroids, antiproliferative agents and diagnostic agents. Therapeutic agents can include a drug or modified form of drug such as prodrugs and analogs. In some forms, the VLPs are used for the delivery of a peptide drug, a dye, an antibody, or antigen-binding fragment of an antibody.

In some forms, the VLPs encapsulate one or more therapeutic, agents.

Examples of therapeutic agents that can be associated with the VLP include, but are not limited to, beta-lactam antibiotics (including penicillins such as ampicillin, cephalosporins selected in turn from cefuroxime, cefaclor, cephalexin, cephydroxil and cepfodoxime proxetil); tetracycline antibiotics (doxycycline and minocycline); microlides antibiotics (azithromycin, erythromycin, rapamycin and clarithromycin); fluoroquinolones (ciprofloxacin, enrofloxacin, ofloxacin, gatifloxacin, levofloxacin) norfloxacin, an antioxidant drug includes N-acetylcysteine (NAC); anti-inflammatory drugs, such as nonsteroidal drugs (e.g., indomethacin, aspirin, acetaminophen, diclofenac sodium and ibuprofen); steroidal anti-inflammatory drug (e.g., dexamethasone); antiproliferative agents (e.g., Paclitaxel (Taxol), QP-2 Vincristin, Methotrexat, Angiopeptin, Mitomycin, BCP 678, Antisense c-myc, ABT 578, Actinomycin-D, RestenASE, 1-Chlor-deoxyadenosin, PCNA Ribozym, and Celecoxib) sirolimus, everolimus and ABT-578), paclitaxel and antineoplastic agents, including alkylating agents (e.g., cyclophosphamide, mechlorethamine, chlorambucil, melphalan, carmustine, lomustine, ifosfamide, procarbazine, dacarbazine, temozolomide, altretamine, cisplatin, carboplatin and oxaliplatin), antitumor antibiotics (e.g., bleomycin, actinomycin D, mithramycin, mitomycin C, etoposide, teniposide, amsacrine, topotecan, irinotecan, doxorubicin, daunorubicin, idarubicin, epirubicin, mitoxantrone and mitoxantrone), antimetabolites (e.g., deoxycoformycin, 6-mercaptopurine, 6-thioguanine, azathioprine, 2-chlorodeoxyadenosine, hydroxyurea, methotrexate, 5-fluorouracil, capecitabine, cytosine arabinoside, azacytidine, gemcitabine, fludarabine phosphate and aspariginase); antimitotic agents (e.g., vincristine, vinblastine, vinorelbine, docetaxel, estramustine); molecularly targeted agents including antibodies, antibody fragments, or carbohydrates/polysaccharides (e.g., imatinib, tretinoin, bexarotene, bevacizumab, gemtuzumab ogomicin and denileukin diftitox); and corticosteroids (e.g., fluocinolone acetonide and methylprednisolone). In some forms, the VLPs encapsidate a checkpoint inhibitor, such as an anti-PD-1 antibody.

In an exemplary form, the VLPs encapsidate a STING agonist in addition to one or more other active agents. For example, in some forms, the VLPs encapsidate a STING agonist in addition to a checkpoint inhibitor.

### B. Excipients, Delivery Vehicles and Devices

The VLPs can be formulated into compositions including suitable excipient for administering the nanoparticles into the body of a subject.

In certain forms, VLPs are formulated in a carrier or excipient suitable for delivery into a subject by injection, for example, via intramuscular (i.m.) intravenous (i.v.), subcutaneous (s.c.), intraperitoneal (i.p.), or via skin scarification, or by a non-injectable route such as pulmonary, topical, or mucosal administration. Typical carriers are saline, phosphate buffered saline, glucose solutions, and other injectable carriers.

Formulations including VLPs with or without delivery vehicles are described. The VLPs can be formulated into pharmaceutical compositions including one or more pharmaceutically acceptable carriers. Pharmaceutical compositions can be formulated for different mechanisms of administration, according to the desired purpose of the VLPs and the intended use. Pharmaceutical compositions formulated for administration by parenteral (intramuscular, intraperitoneal, intravenous (IV), intraocular or subcutaneous injection), topical or transdermal (either passively or using iontophoresis or electroporation) routes of administration or using bioerodible inserts are described.

### 1. Parenteral Administration

In some forms, VLPs are formulated for administration in an aqueous solution, by parenteral injection. The formulation may also be in the form of a suspension or emulsion. In general, pharmaceutical compositions are provided including effective amounts of an active agent, targeting moiety, and optional a delivery vehicle and optionally include pharmaceutically acceptable diluents, preservatives, solubilizers, emulsifiers, and/or carriers.

Such compositions include the diluents sterile water, buffered saline of various buffer content (e.g., Tris-HCl, acetate, phosphate), pH and ionic strength and optionally additives such as detergents and solubilizing agents (e.g., TWEEN® 20, TWEEN® 80 also referred to as polysorbate 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

### 2. Pulmonary, Topical and Mucosal Administration

Compositions of VLPs can be formulated for application topically, by instillation or by inhalation. In some forms, VLPs are formulated for administration to the mucosa, such as the lungs, mouth, eyes, lungs, nasal, oral (sublingual, buccal), vaginal, or rectal mucosa. Formulations for administration to the mucosa will typically be spray dried drug particles, which may be incorporated into a tablet, gel, capsule, suspension or emulsion. Standard pharmaceutical excipients are available from any formulator.

In some forms, the VLPs are formulated for delivery to the skin, for example, by direct application to the surface of diseased, or damaged or ruptured skin. Therefore, in some forms, VLPs are formulated for delivery to a wound or site of surgery. Compositions formulated for topical delivery can include one or more penetration enhancers.

In one form, the VLPs are formulated for pulmonary delivery, such as intranasal administration or oral inhalation. The respiratory tract is the structure involved in the exchange of gases between the atmosphere and the blood stream. The upper and lower airways are called the conducting airways. The terminal bronchioli divide into respiratory bronchiole, which then lead to the ultimate respiratory zone, the alveoli, or deep lung. The deep lung, or alveoli, is the primary target of inhaled therapeutic aerosols for systemic drug delivery. Therapeutic agents that are active in the lungs can be administered systemically and targeted via pulmonary absorption. The term aerosol refers to any preparation of a fine mist of particles, which can be in solution or a suspension, whether or not it is produced using a propellant. Aerosols can be produced using standard techniques, such as ultra-sonication or high-pressure treatment.

Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into a solution, e.g., water or isotonic saline, buffered or un-buffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. One skilled in the art can readily determine a suitable saline content and pH for an innocuous aqueous solution for nasal and/or upper respiratory administration.

Compositions can be delivered to the lungs while inhaling and traverse across the lung epithelial lining to the blood stream when delivered either as an aerosol or spray dried particles having an aerodynamic diameter of less than about 5 microns.

Dry powder formulations (“DPFs”) with large particle size have improved flowability characteristics, such as less aggregation, easier aerosolization, and potentially less phagocytosis. Dry powder aerosols for inhalation therapy are generally produced with mean diameters primarily in the range of less than 5 microns, although a preferred range is between one and ten microns in aerodynamic diameter. Large “carrier” particles (containing no drug) have been co-delivered with therapeutic aerosols to aid in achieving efficient aerosolization among other possible benefits. A wide range of mechanical devices designed for pulmonary delivery of therapeutic products can be used, including, but not limited to, nebulizers, metered dose inhalers, and powder inhalers, all of which are familiar to those skilled in the art.

### C. Additional Active Agents

In some forms, the VLP is administered as a composition in combination with a conventional therapeutic agent used for treatment of the disease or condition being treated. Therefore, in some forms, the VLPs are formulated for administration together with one or more additional active agents.

Conventional therapeutics agents are known in the art and can be determined by one of skill in the art based on the disease or disorder to be treated. For example, if the disease or condition is cancer, the VLP can be co-administered with a chemotherapeutic drug; or if the disease or condition is a bacterial infection, the VLP can be co-administered with an antibiotic.

As set forth in the Examples, the administration of VLPs together with one or more STING agonists, and checkpoint inhibitors, gave rise to cancer cell killing. Therefore, in some forms, the compositions of VLPs are administered in combination with a PD-1 antagonist, STING agonist, or a combination thereof.

### 1. Checkpoint Inhibitors

In some forms, compositions of VLPs are administered in combination with a PD-1 antagonist.

Activation of T cells normally depends on an antigen-specific signal following contact of the T cell receptor (TCR) with an antigenic peptide presented via the major histocompatibility complex (MHC) while the extent of this reaction is controlled by positive and negative antigen-independent signals emanating from a variety of co-stimulatory molecules. The latter are commonly members of the CD28/B7 family. Conversely, Programmed Death-1 (PD-1) is a member of the CD28 family of receptors that delivers a negative immune response when induced on T cells. Contact between PD-1 and one of its ligands (B7-H1 or B7-DC) induces an inhibitory response that decreases T cell multiplication and/or the strength and/or duration of a T cell response. Suitable PD-1 antagonists are described in U.S. Pat. Nos. 8,114,845, 8,609,089, and 8,709,416, and include compounds or agents that either bind to and block a ligand of PD-1 to interfere with or inhibit the binding of the ligand to the PD-1 receptor, or bind directly to and block the PD-1 receptor without inducing inhibitory signal transduction through the PD-1 receptor.

In some forms, the PD-1 receptor antagonist binds directly to the PD-1 receptor without triggering inhibitory signal transduction and also binds to a ligand of the PD-1 receptor to reduce or inhibit the ligand from triggering signal transduction through the PD-1 receptor. By reducing the number and/or amount of ligands that bind to PD-1 receptor and trigger the transduction of an inhibitory signal, fewer cells are attenuated by the negative signal delivered by PD-1 signal transduction and a more robust immune response can be achieved.

It is believed that PD-1 signaling is driven by binding to a PD-1 ligand (such as B7-H1 or B7-DC) in close proximity to a peptide antigen presented by major histocompatibility complex (MHC) (see, for example, Freeman, Proc. Natl. Acad. Sci. U. S. A, 105: 10275-10276 (2008)).

Therefore, proteins, antibodies or small molecules that prevent co-ligation of PD-1 and TCR on the T cell membrane are also useful PD-1 antagonists.

In preferred forms, the PD-1 receptor antagonists are small molecule antagonists or antibodies that reduce or interfere with PD-1 receptor signal transduction by binding to ligands of PD-1 or to PD-1 itself, especially where co-ligation of PD-1 with TCR does not follow such binding, thereby not triggering inhibitory signal transduction through the PD-1 receptor.

Other PD-1 antagonists contemplated by the methods of this invention include antibodies that bind to PD-1 or ligands of PD-1, and other antibodies.

Suitable anti-PD-1 antibodies include, but are not limited to, those described in the following publications: PCT/IL03/00425 (Hardy et al, WO/2003/099196), PCT/JP2006/309606 (Korman et al, WO/2006/121168), PCT/US2008/008925 (Li et al, WO/2009/014708), PCT/JP03/08420 (Honjo et al, WO/2004/004771), PCT/JP04/00549 (Honjo et al, WO/2004/072286), PCT/IB2003/006304 (Collins et al, WO/2004/056875), PCT/US2007/088851 (Ahmed et al, WO/2008/083174), PCT/US2006/026046 (Korman et al, WO/2007/005874), PCT/US2008/084923 (Terrett et al, WO/2009/073533), Berger et al, Clin. Cancer Res., 14:30443051 (2008).

A specific example of an anti-PD-1 antibody is MDX-1106 (see Kosak, US 20070166281 (pub. 19 Jul. 2007) at par. 42), a human anti-PD-1 antibody, preferably administered at a dose of 3 mg/kg.

Exemplary anti-B7-H1 antibodies include, but are not limited to, those described in the following publications: PCT/US06/022423 (WO/2006/133396, pub. 14 Dec. 2006), PCT/US07/088851 (WO/2008/083174, pub. 10 Jul. 2008) US 2006/0110383 (pub. 25 May 2006)

A specific example of an anti-B7-H1 antibody is MDX-1105 (WO/2007/005874, published 1 1 Jan. 2007)), a human anti-B7-Hl antibody.

For anti-B7-DC antibodies see U.S. Pat. Nos. 7,411,051, 7,052,694, 7,390,888, and U.S. Published Application No. 2006/0099203.

The antibody can be a bi-specific antibody that includes an antibody that binds to the PD-1 receptor bridged to an antibody that binds to a ligand of PD-1, such as B7-H1. In some forms, the PD-1 binding portion reduces or inhibits signal transduction through the PD-1 receptor.

Other exemplary PD-1 receptor antagonists include, but are not limited to B7-DC polypeptides, including homologs and variants of these, as well as active fragments of any of the foregoing, and fusion proteins that incorporate any of these. In a preferred form, the fusion protein comprises the soluble portion of B7-DC coupled to the Fc portion of an antibody, such as human IgG, and does not incorporate all or part of the transmembrane portion of human B7-DC.

The PD-1 antagonist can also be a fragment of a mammalian B7-H1, preferably from mouse or primate, preferably human, wherein the fragment binds to and blocks PD-1 but does not result in inhibitory signal transduction through PD-1. The fragments can also be part of a fusion protein, for example an Ig fusion protein.

Other useful polypeptides PD-1 antagonists include those that bind to the ligands of the PD-1 receptor. These include the PD-1 receptor protein, or soluble fragments thereof, which can bind to the PD-1 ligands, such as B7-H1 or B7-DC, and prevent binding to the endogenous PD-1 receptor, thereby preventing inhibitory signal transduction. B7-H1 has also been shown to bind the protein B7.1 (Butte et al, Immunity, Vol. 27, pp. 1 11-122, (2007)). Such fragments also include the soluble ECD portion of the PD-1 protein that includes mutations, such as the A99L mutation, that increases binding to the natural ligands (Molnar et al, PNAS, 105: 10483-10488 (2008)). B7-1 or soluble fragments thereof, which can bind to the B7-H1 ligand and prevent binding to the endogenous PD-1 receptor, thereby preventing inhibitory signal transduction, are also useful.

PD-1 and B7-H1 anti-sense nucleic acids, both DNA and RNA, as well as siRNA molecules can also be PD-1 antagonists. Such anti-sense molecules prevent expression of PD-1 on T cells as well as production of T cell ligands, such as B7-H1, PD-L1 and/or PD-L2. For example, siRNA (for example, of about 21 nucleotides in length, which is specific for the gene encoding PD-1, or encoding a PD-1 ligand, and which oligonucleotides can be readily purchased commercially) complexed with carriers, such as polyethyleneimine (see Cubillos-Ruiz et al, J. Clin. Invest. 119(8): 2231-2244 (2009), are readily taken up by cells that express PD-1 as well as ligands of PD-1 and reduce expression of these receptors and ligands to achieve a decrease in inhibitory signal transduction in T cells, thereby activating T cells.

### 2. Cyclic Dinucleotides

In some forms, the VLPs are formulated for administration as a composition together with one or more STING agonists.

For example, in some forms, the VLPs are administered together with functional nucleic acids that encode a cyclic dinucleotide. Cyclic dinucleotides bind directly to the STING adaptor protein, resulting in production of IFN-β (Zhang, et al., Mol Cell., 51(2):226-35 (2013). doi: 10.1016/j.molcel.2013.05.022.). Several canonical and noncanonical dinucleotides are known in the art, and include, but are not limited to, 2′3′-cGAMP, 2′3′-cGAMP, 3′3′-cGAMP, c-di-AMP, c-di-GMP, cAIMP (CL592), cAIMP Difluor (CL614), cAIM(PS)2 Difluor (Rp/Sp) (CL656), 2′2′-cGAMP, 2′3′-cGAM(PS)2 (Rp/Sp), 3′3′-cGAMP Fluorinated, c-di-AMP Fluorinated, 2′3′-c-di-AMP, 2′3′-c-di-AM(PS)2 (Rp,Rp), 2′3′-c-di-AM(PS)2 (Rp,Rp), c-di-GMP Fluorinated, 2′3′-c-di-GMP, c-di-IMP, DMXAA.

### 3. Adjuvants

The VLP compositions and methods and include one or more adjuvants in the same or different compositions, administered together or separately from the VLP composition.

The adjuvant may be without limitation alum (e.g., aluminum hydroxide, aluminum phosphate); saponins purified from the bark of the Q. saponaria tree such as QS21 (a glycolipid that elutes in the 21st peak with HPLC fractionation; Antigenics, Inc., Worcester, Mass.); poly[di(carboxylatophenoxy)phosphazene (PCPP polymer; Virus Research Institute, USA), Flt3 ligand, Leishmania elongation factor (a purified Leishmania protein; Corixa Corporation, Seattle, Wash.), ISCOMS (immunostimulating complexes which contain mixed saponins, lipids and form virus-sized particles with pores that can hold antigen; CSL, Melbourne, Australia), Pam3Cys, SB-AS4 (SmithKline Beecham adjuvant system #4 which contains alum and MPL; SBB, Belgium), non-ionic block copolymers that form micelles such as CRL 1005 (these contain a linear chain of hydrophobic polyoxypropylene flanked by chains of polyoxyethylene, Vaxcel, Inc., Norcross, Ga.), and Montanide IMS (e.g., IMS 1312, water-based nanoparticles combined with a soluble immunostimulant, Seppic).

Adjuvants may be TLR ligands, such as those discussed above. Adjuvants that act through TLR3 include without limitation double-stranded RNA. Adjuvants that act through TLR4 include without limitation derivatives of lipopolysaccharides such as monophosphoryl lipid A (MPLA; Ribi ImmunoChem Research, Inc., Hamilton, Mont.) and muramyl dipeptide (MDP; Ribi) andthreonyl-muramyl dipeptide (t-MDP; Ribi); OM-174 (a glucosamine disaccharide related to lipid A; OM Pharma SA, Meyrin, Switzerland). Adjuvants that act through TLR5 include without limitation flagellin. Adjuvants that act through TLR7 and/or TLR8 include single-stranded RNA, oligoribonucleotides (ORN), synthetic low molecular weight compounds such as imidazoquinolinamines (e.g., imiquimod (R-837), resiquimod (R-848)). Adjuvants acting through TLR9 include DNA of viral or bacterial origin, or synthetic oligodeoxynucleotides (ODN), such as CpG ODN. Another adjuvant class is phosphorothioate containing molecules such as phosphorothioate nucleotide analogs and nucleic acids containing phosphorothioate backbone linkages. Adjuvants can be STING agonist such as 2′3′-cGAMP or any of the other mentioned else herein.

The adjuvant can also be oil emulsions (e.g., Freund's adjuvant); saponin formulations; virosomes and viral-like particles; bacterial and microbial derivatives; immunostimulatory oligonucleotides; ADP-ribosylating toxins and detoxified derivatives; alum; BCG; mineral-containing compositions (e.g., mineral salts, such as aluminium salts and calcium salts, hydroxides, phosphates, sulfates, etc.); bioadhesives and/or mucoadhesives; microparticles; liposomes; polyoxyethylene ether and polyoxyethylene ester formulations; polyphosphazene; muramyl peptides; imidazoquinolone compounds; and surface active substances (e.g. lysolecithin, pluronic polyols, polyanions, peptides, oil emulsions, keyhole limpet hemocyanin, and dinitrophenol).

Adjuvants may also include immunomodulators such as cytokines, interleukins (e.g., IL-1, IL-2, IL-4, IL-5, IL-6, IL-7, IL-12, etc.), interferons (e.g., interferon-.gamma.), macrophage colony stimulating factor, and tumor necrosis factor.

In some embodiments, the compositions and methods are adjuvant-free, or free from adjuvant that is not package with the VLPs.

### III. Methods of Use

It has been established that VLPs displaying antigen and DC ligands provide an effective, biocompatible and non-toxic vehicle for the activation of DCs and generation of antigen-specific immunity in vivo. Methods of activating antigen presenting cells (APCs) are provided.

The methods typically administer synthetic Virus Like Particles (VLPs) for antigen-specific activation of dendritic cells, including (a) a DC-SIGN ligand; and (b) one or more peptide antigen(s) to a subject, wherein the DC-SIGN ligand and the antigen(s) are present at the outer surface of the synthetic particle and generate an APC-initiated immune response to the antigen in the subject. Typically, the subject has, or is at risk of having a disease or disorder, such as an infectious disease. In some forms, the antigen is derived from or stimulates an immune response to a pathogen associated with the disease, and the antigen stimulates an immune response to the pathogen in the subject. In particular forms, the subject has, or is at risk of having cancer, the antigen is a tumor antigen, and the antigen stimulates an immune response to the tumor antigen in the subject.

The described VLPs target APCs and are readily taken up into the APC in vivo to efficiently deliver enclosed antigen and optionally additional active agents to the APCs, for the development of specific Th-1 immune responses in a subject. For example, when VLPs are trans-located into the cytoplasm of the host APC, the encapsulated agents (e.g., nucleic acid) are deposited into the cell. The antigen is processed by the APC and gives rise to presentation of the VLP-bound antigen at the surface of the APC in the context of MIC class-I as well as MIC class-II, whilst encapsulated agents function to enhance innate immune processes, as a form of self-adjuvanting immunogen.

Therefore, methods of using VLPs to stimulate both CD8+ T cells and CD4+ T cells are provided. In some forms, the methods can include administering to a subject an effective amount of a composition including VLPs having bound thereto two species of peptide antigen directed to the same or different pathogens or tumors and one or more CLR ligand for targeting to and activating professional antigen-presenting cells.

The VLPs can induce a biological effect in the cells of the recipient, such as an immune-modulatory effect. For example, the VLPs can be used to stimulate an immune response to the VLP-associated peptide antigen in the subject. In some forms, the VLPs are also safe and effective delivery vehicles for encapsidated active agents, such as immunostimulatory agents for enhancing the efficacy of antigen-specific immune responses.

Methods to prevent or reduce one or more diseases or disorders in a subject are also provided. Typically, the methods include administering to a subject an effective amount of the VLPs either alone, or in combination with one or more additional active agents to reduce or prevent one or more diseases or disorders in the subject. In a preferred form, the methods treat or prevent a cancer in the subject.

### A. Methods of Delivering Peptides Antigens to APCs

It has been established that VLPs having surface-bound C-type lectin receptors (CLRs) effective delivery bound peptide antigen and encapsidated agents, including bacterial nucleic acids to the interior of cells, leading to enhanced antigen-specific immune responses in vivo. Typically, the antigen-specific cellular immune responses generated when two or more species of antigen are attached to the VLP are greater than when a single species of antigen is attached to the VLP. Typically, the immune responses generated in a host are enhanced when the VLPs encapsidate an adjuvant, such as immunostimulatory nucleic acids.

The VLPs are rapidly internalized into APCs of a subject in vivo. Typically, carbohydrates at the surface of the VLP coordinate uptake of the VLP by DCs, for example, by binding to DC-SIGN on the surface of DCs. It may be that VLPs are internalized into the cell by generalized endocytosis. The antigen bound to the VLPs is processed and displayed by MHC class I and MHC class II receptors, to stimulate CD8+ T cell responses and simultaneous engagement of MHC class II receptors by CD4+ T cells. Typically, the delivery requires contact and internalization of the VLPs by the target APCs. Internalization can occur through one or more different mechanisms. The contacting between the VLPs and target cells can be induced occur in vivo or in vitro. Generally, the contacting occurs in vivo.

Therefore, in some forms, the VLPs are administered to a subject. In some forms, the VLPs are systemically administered to a subject. In other forms, the APCs are administered to a specific bodily location of the subject.

Pharmaceutical compositions including VLPs can be administered in a variety of manners, depending on whether local or systemic administration is desired, and depending on the area to be treated. Injectable formulations can be prepared in conventional forms, either as liquid solutions or suspensions, solid forms suitable for solution of suspension in liquid prior to injection, or as emulsions. In certain forms, the compositions are administered locally, for example, by injection directly into a site to be treated. In some forms, local delivery can reduce side effects or toxicity associated with systemic delivery and can result in enhanced outcome due to an increased localized dose.

The compositions can be injected or otherwise administered directly to one or more surgical sites. Typically, local injection causes an increased localized concentration of the VLP compositions which is greater than that which can be achieved by systemic administration.

Compositions of VLPs can be administered during a period before, during, or after onset of symptoms of a disease, or any combination of periods before, during or after onset of one or more disease symptoms. For example, the subject can be administered one or more doses of the composition every 1, 2, 3, 4, 5, 6 7, 14, 21, 28, 35, or 48 days prior to the onset of disease symptoms, (i.e., prior to the predicted onset). The subject can be administered one or more doses of the composition every 1, 2, 3, 4, 5, 6, 7, 14, 21, 28, 35, or 48 days after the onset of disease symptoms. In some forms, the multiple doses of the compositions are administered before an improvement in disease condition is evident. For example, in some forms, the subject receives 1, 2, 3, 4, 5, 6 7, 14, 21, 28, 35, or 48 doses over a period of 1, 2, 3, 4, 5, 6 7, 14, 21, 28, 35, or 48 days or weeks before an improvement in the disease or condition is evident.

Thus, compositions including one or more VLPs can be administered at different times in relation to a diagnosis, prognosis, surgery or injury depending on the desired effects of the nucleic acids or polypeptides that are delivered to the target cells. The timing of commencement of administration of the VLPs should be determined based upon the needs of the subject, and can vary accordingly.

In some forms, a single dose of VLPs is delivered to a subject as one or more bolus doses, i.e., to raise the blood concentration of the VLPs, or the blood concentration of the payload of the VLPs to a desired level. The dose of VLPs can be given by any means, such as via injection. In a particular form, a dose is given together with, prior to, or after the administration of one or more additional active agents, e.g., in other dosage forms.

In particular forms, the VLPs are delivered to inoculate a subject from a disease, such as an infectious disease, or a cancer. Therefore, in some forms, the VLPs are used to prevent or treat cancer in a subject in need thereof. When the VLPs are used to treat or prevent cancer in a subject, the VLPs typically display one or more cancer antigens. In preferred forms, the VLPs include two distinct antigens that raise CD8+ T cell responses and or CD4+ T cell responses to a cancer in a subject.

In other forms, the VLP are used to prime or otherwise prepare APC's in vitro or ex vivo. Subsequent, the APC's can be administered to subject in need thereof in a therapeutically effective amount, e.g., to treat cancer or the other diseases or conditions mentioned herein as targets of in vivo therapy. Additional or alternatively, the primed or otherwise prepared APC's can be further used in vitro or ex vivo to primer or otherwise activate T cells for adaptive T cell therapy. Thus, in some embodiments, in vitro or ex vivo T cells activated by APCs treated with disclosed VLPs are administered to subject in need thereof in a therapeutically effective amount, e.g., to treat cancer or the other diseases or conditions mentioned herein as targets of in vivo therapy.

### B. Vaccination

In some forms, VLPs can deliver antigenic proteins to a subject to stimulate desired immune responses in the subject. The delivery of antigen via the VLPs confers protective immunity to infectious agents such as viruses and bacteria. Typically, the VLPs deliver antigen more effectively than the same antigen alone. For example, peptide antigen delivered to APCs in a subject can drive antigen-specific T cell immunity in a subject to a greater extent than the same amount of the same peptide antigen delivered in the absence of the described VLP.

Methods for vaccination using peptide antigens attached to the described VLPs are provided which allow potent and persistent presentation of antigen to the immune system.

Experiments conducted with the VLPs including antigens encoding the OVA peptide (discussed below), demonstrate that VLPs can effectively deliver a desired antigen to APCs and generate strong immune responses specifically against cancers designed to express the OVA peptide.

Other antigens could include proteins from pathogenic microbes such as viruses, bacteria, fungi, protozoa, as well as other cancer antigens/neoantigens. Therefore, the VLPs can serve as a safe and effective platform for the delivery vaccine agents targeting many different pathogens, together with encapsidated active agents such as immunostimulatory nucleic acids.

Typically, VLP-mediated delivery of exogenous antigen to the cells of a subject results in the production of antibodies and other biomolecules capable of recognizing and neutralizing the antigen. Antigen that has been arrayed on the surface of antigen-presenting cells (APC) can be presented to a “helper” T cell, such as an antigen-specific naive CD4+ T cell. Such presentation delivers a signal via the T cell receptor (TCR) that directs the T cell to initiate an immune response that will be specific to the presented.

Therefore, the VLPs can be used to initiate, moderate or enhance a humoral and/or cellular immunity to the antigen. For example, the VLPs deliver exogenous proteins in an amount effective to induce, enhance or otherwise moderate the biological activities of immune cells, such as macrophages, B-cells, T-cells, dendritic cells and NK cells.

In some forms, administration of the VLPs including antigen to a subject confers immunity to the antigen to the subject. Immunity can manifest in the production of a reservoir of memory T cells (i.e., memory CD8+ T cells) and/or antigen-specific B cells in the subject sufficient to provide rapid immune cellular and/or humoral immune responses to repeat exposure of the antigen. Preferably, administration of the VLPs including antigen confers protection against cancer or an infection or disease caused by the organism(s) from which the antigen is derived.

Typically, administration of the VLPs including antigen to a subject enhances the uptake and delivery of antigen to the antigen presenting cells of the subject relative to administration of equal amounts of the antigen or nucleic acid encoding the antigen alone. Therefore, administration of antigen to a subject via the VLPs can enhance the immune response to the antigen in the subject relative to administration of equal amounts of the antigen or nucleic acid encoding the antigen alone. For example, VLPs can increase, prolong or otherwise enhance presentation of the encoded antigen at the surface of antigen presenting cells of the subject.

Vaccines can be administered prophylactically or therapeutically. Vaccines can also be administered according to a vaccine schedule. A vaccine schedule is a series of vaccinations, including the timing of all doses. Many vaccines require multiple doses for maximum effectiveness, either to produce sufficient initial immune response or to boost response that fades over time. Vaccine schedules are known in the art and are designed to achieve maximum effectiveness. The adaptive immune response to one or more antigen delivered in the VLPs can be monitored using methods known in the art to measure the effectiveness of the vaccination protocol.

In some forms, VLPs deliver exogenous proteins and/or nucleic acids to a subject and stimulate immune responses specifically to antigen that is biologically-processed by the host cells, for example, processed for presentation by the APC in the context of MHC.

### 1. Vaccination Strategies for Multiple Antigens

As described above, VLPs can include two or more different peptide antigens. The two or more different peptide antigens can be engineered to express the same or different immunogenic antigens. The antigens can be exogenous or endogenous.

In some forms, vaccines include combinations of different species of antigens, each with different MHC presentation/antigen display kinetics. By “multiplexing” antigens with different kinetics, VLP vaccines can engineered to induce the expression of two different antigens, e.g., a first antigen that is processed by the APC for presentation via the MHC class I pathway, and a second antigen that is processed by the APC for presentation via the MHC class II pathway following uptake by the APC. See, e.g., Neefjes, “Towards a systems understanding of MHC class I and MHC class II antigen presentation. Nat Rev Immunol,” 11, 823-836 (2011) doi.org/10.1038/nri3084, which is specifically incorporated by reference herein in its entirety. The “multiplexed” vaccines can include a mixture of distinct VLPs, each enclosing a single antigen species, or alternatively each VLP can be engineered to include more than one antigen species.

Therefore, methods of administering a multiplicity of antigens attached to a multiplicity of VLPs are provided. The multiplicity of VLPs can include two or more different VLPs, each displaying a different antigen species at the surface of the VLP. Therefore, methods of administering two or more different antigen species, independently bound to VLPs displaying the antigen at the surface of the VLP are provided.

When VLPs are engineered to include one species of antigen, vaccines can be designed by mixing a desired amount of each VLP to create a combined VLP vaccine, having the desired combination of antigens to produce a desired immune response. For example, in an exemplary form, the composition includes two species of VLP, each presenting a distinct antigen derived from the same cancer or pathogen, but with distinct structural features. In an exemplary form, one antigen is engineered for presentation by the APC via MHC class I and a second pr further VLP includes a second or further antigen engineered for presentation by the APC via MHC class II.

Typically, the VLP-based vaccines are self-limiting and only produce antigens for a finite amount of time until the host eliminates the VLPs and all vaccine products ale cleared by the body. Antigen processing and presentation occurs in host cell cytoplasm, and the genetic material in the nucleus of the cell is never manipulated.

In some forms, the VLPs are engineered to include more than one encapsulated active agent. For example, VLPs can include nucleic acids designed to stimulate TLR activity within a subject and thereby enhance immune activation by the one or more antigen(s).

In some forms, the VLPs deliver cargo, such as adjuvants, to a subject to provide immunostimulatory effects to the antigen in the subject.

In other forms, the VLP vehicles can be designed to enter cells and deliver therapeutic, prophylactic and diagnostic agents to the APCs in vivo.

In some forms, when VLPs are used to deliver antigen to the APCs of a subject, a smaller molar amount of the antigen is required to produce the same antigen-specific immune response in the subject as compared to the molar amount of antigen delivered alone/without the VLPs to produce the same antigen-specific immune response. Generally, a smaller amount of VLPs including antigen can be required to produce an antigen-specific immune response in a subject as compared to the amount of protein antigen or mRNA encoding the same antigen. Therefore, VLPs including antigen can be used to induce an antigen-specific immune response in a subject that reduces any undesirable effects associated with the introduction of the antigen into a subject.

### 2. Pathogens/Diseases to be Vaccinated Against

The VLPs deliver protein antigen and immunostimulatory nucleic acid to a subject in an amount effective to vaccinate the subject from one or more diseases and disorders. Therefore, VLPs can serve as a vaccination platform for a wide variety of microbial pathogens, such as bacterial, viral, fungal and protozoan pathogens.

In some forms, the target of the vaccine could be a type of cancer cell as a cancer treatment. Alternately, the target could be any of a large number of microbial pathogens. Exemplary diseases that can be vaccinated against include disease for which vaccines are currently available. Alternatively or in addition, VLPs can serve as a platform for inducing immunological tolerance to a subject to one or more allergens, such as food allergens and environmental allergens.

a. Cancer

In certain forms, VLPs can be used to immunize a subject against cancer. The VLPs can be administered to a subject diagnosed with cancer (i.e., as a therapeutic vaccine), or to a subject having a predisposition or risk of developing cancer (i.e., as a prophylactic vaccine). In some forms, the compositions of VLPs are administered to a cancer patient in addition to one or more additional therapeutic agents.

To create an ideal cancer therapy, bioinformatics is used to sequence each patient's unique tumor exome to identify neoantigens. Then, corresponding mRNAs of these neoantigens are used to generate the antigens necessary to create immunity. Finally, these mRNAs are delivered using an adjuvant-free nanotechnology delivery platform capable of activating both the cytotoxic T cell and humoral arms of the immune system in order to create durable and long-term protection against new tumor growth and metastases.

In some forms, the VLPs include one or more tumor antigens. The VLPs can be used to provide immunity and therapeutic activity against tumor cells and non-tumor cells located within a tumor or a tumor environment. VLPs can be formulated to provide protective and/or therapeutic activity against solid tumors and cancers of the blood. Exemplary tumor cells include, but are not limited to, tumor cells of cancers, including leukemias including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as, but not limited to, Hodgkin's disease, non-Hodgkin's disease; multiple myelomas such as, but not limited to, smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as, but not limited to, bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors including, but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including, but not limited to, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer, including, but not limited to, pheochromocytom and adrenocortical carcinoma; thyroid cancer such as but not limited to papillary or follicular thyroid cancer, medullary thyroid cancer and anaplastic thyroid cancer; pancreatic cancer, including, but not limited to, insulinoma, gastrinoma, glucagonoma, vipoma, somatostatin-secreting tumor, and carcinoid or islet cell tumor; pituitary cancers including, but not limited to, Cushing's disease, prolactin-secreting tumor, acromegaly, and diabetes insipius; eye cancers including, but not limited to, ocular melanoma such as iris melanoma, choroidal melanoma, and cilliary body melanoma, and retinoblastoma; vaginal cancers, including, but not limited to, squamous cell carcinoma, adenocarcinoma, and melanoma; vulvar cancer, including, but not limited to, squamous cell carcinoma, melanoma, adenocarcinoma, basal cell carcinoma, sarcoma, and Paget's disease; cervical cancers including, but not limited to, squamous cell carcinoma, and adenocarcinoma; uterine cancers including, but not limited to, endometrial carcinoma and uterine sarcoma; ovarian cancers including, but not limited to, ovarian epithelial carcinoma, borderline tumor, germ cell tumor, and stromal tumor; esophageal cancers including, but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers including, but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers including, but not limited to, hepatocellular carcinoma and hepatoblastoma, gallbladder cancers including, but not limited to, adenocarcinoma; cholangiocarcinomas including, but not limited to, papillary, nodular, and diffuse; lung cancers including, but not limited to, non-small cell lung cancer, squamous cell carcinoma (epidermoid carcinoma), adenocarcinoma, large-cell carcinoma and small-cell lung cancer; testicular cancers including, but not limited to, germinal tumor, seminoma, anaplastic, classic (typical), spermatocytic, nonseminoma, embryonal carcinoma, teratoma carcinoma, choriocarcinoma (yolk-sac tumor), prostate cancers including, but not limited to, adenocarcinoma, leiomyosarcoma, and rhabdomyosarcoma; penal cancers; oral cancers including, but not limited to, squamous cell carcinoma; basal cancers; salivary gland cancers including, but not limited to, adenocarcinoma, mucoepidermoid carcinoma, and adenoidcystic carcinoma; pharynx cancers including, but not limited to, squamous cell cancer, and verrucous; skin cancers including, but not limited to, basal cell carcinoma, squamous cell carcinoma and melanoma, superficial spreading melanoma, nodular melanoma, lentigo malignant melanoma, acral lentiginous melanoma; kidney cancers including, but not limited to, renal cell cancer, adenocarcinoma, hypernephroma, fibrosarcoma, transitional cell cancer (renal pelvis and/or uterer); Wilms' tumor; bladder cancers including, but not limited to, transitional cell carcinoma, squamous cell cancer, adenocarcinoma, carcinosarcoma. Cancers that can be prevented, treated or otherwise diminished by the VLPs include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma, papillary adenocarcinomas, and gastric cancer (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America). In some forms, VLPs can be used to immunize a subject against one or more cancers for which no alternative vaccine is available.

b. Infectious Diseases

VLPs can deliver antigens to the APCs of a subject in an amount effective to vaccinate the subject from one or more infectious diseases caused by a wide variety of microbial pathogens, such as bacterial, viral, fungal and protozoan pathogens.

In some forms, the target of the vaccine could be any of a large number of microbial pathogens. Exemplary diseases that can be vaccinated against include disease for which vaccines are currently available, including Anthrax; Diseases (e.g., cervical cancer, cancer of the esophagus) caused by Human Papillomavirus (HPV); Diphtheria; Hepatitis A; Hepatitis B; Haemophilus influenzae type b (Hib); Influenza viruses (Flu); Japanese encephalitis (JE); Lyme disease; Measles; Meningococcal; Monkeypox; Mumps; Pertussis; Pneumococcal; Polio; Rabies; Rotavirus; Rubella; Shingles (Herpes Zoster); Smallpox; Tetanus; Toxoplasmosis; Typhoid; Tuberculosis (TB); Varicella (Chickenpox); Yellow Fever.

In some forms, VLP can be used to immunize a subject against an infectious disease or pathogen for which no alternative vaccine is available, such as diseases including, but not limited to, malaria, streptococcus, Ebola Zaire, HIV, Herpes virus, hepatitis C, Middle East Respiratory Syndrome (MERS), Sleeping sickness, Severe Acute Respiratory Syndrome (SARS), rhinovirus, chicken pox, hendra, NIPA virus, Zika Virus, and others.

In some forms, the disease is a pathogen that infects non-mammalian subjects, such as birds. Exemplary avian subjects include domesticated birds (i.e., poultry), such as chickens, ducks, geese, pheasants and other commercial fowl, or pet birds such as parakeets and parrots. For example, bacterial hybrid vectors can be useful to vaccinate birds against Infectious Bursal Disease (IBD). IBD, also known as Gumboro disease, a viral disease affecting the Bursa of Fabricius of young chickens. Other diseases and disorders of poultry that can be vaccinated for using the described bacterial hybrid vectors include Influenza, Ranikhet; Mareks disease, fowl pox, fowl cholera, egg drope syndrome, infectious coryza, coccidiosis, avian encephalitis, avian influenza, chicken infectious anemia and salmonella.

c. Allergies

In certain forms, VLPs can be used to immunize a subject against an allergen. The VLPs can be administered to a subject diagnosed with an allergy or to a subject having a predisposition to an allergy. In some forms, the compositions of VLPs are administered to a patient having an allergy in addition to one or more additional therapeutic agents.

Allergies are abnormal reactions of the immune system that occur in response to otherwise harmless substances. An allergy is a type of immune reaction in which the immune system responds to foreign microorganisms or particles by producing specific antibodies capable of binding to allergens such as pollen, dust, animal hairs, etc. Allergic reactions that can be treated include delayed hypersensitivity reactions and immediate hypersensitivity reactions.

Allergies that can be treated include allergic responses in the skin, such as dermatitis, the upper airways and eyes, such as allergic rhinitis, hay fever, asthma, and conjunctivitis (pink eye) in the gastrointestinal tract, such as food allergies, and blood stream, such as urticaria and hives, angioedema, anaphylaxis, or atopic dermatitis.

### C. Combinations

In some forms, the methods administer VLPs to a subject in need thereof alone. In other forms, the methods administer VLPs to a subject in need thereof in combination with one or more additional active agent(s), as part of a therapeutic or prophylactic treatment regime.

The VLPs can be administered on the same day, or a different day than the second or further active agent. For example, compositions including VLPs can be administered on the first, second, third, or fourth day, or combinations thereof.

The term “combination” or “combined” is used to refer to either concomitant, simultaneous, or sequential administration of two or more agents. Therefore, the combinations can be administered either concomitantly (e.g., as an admixture), separately but simultaneously (e.g., via separate intravenous lines into the same subject), or sequentially (e.g., one of the compounds or agents is given first followed by the second).

In some forms, the additional prophylactic or therapeutic agents can be vaccines for a specific antigen. The antigen can be the same or different to that encoded by the VLPs.

In some forms, the VLPs are useful as an agent to enhance the immune response to an antigen in a subject relative to the immune response raised to the same antigen in the absence of the VLP delivery vehicles.

When VLPs are used to induce an immune response, for example, to one or more antigens or allergens encoded by RNA encapsulated within the VLPs, the administration of an effective amount of the VLPs does not require the co-administration of an adjuvant to elicit the desired immune response. Therefore, in some forms, the VLPs are administered in the absence of an adjuvant, or immuno-stimulatory molecule.

In some forms, the VLPs are administered to a subject as part of a therapeutic or prophylactic regimen together with additional active agents, such as therapeutic agents, in an amount sufficient for the treatment of the subject for a disease or disorder. In certain forms, when the VLPs are administered to treat cancer in a subject, VLPs are administered in combination with one or more additional anti-cancer and/or immunomodulating agents. For example, any of the agents mentioned above as being those that can be encapsidated in the VLPs may additionally or alternatively be administer unencapsidated, e.g., through the same or different means as the VLPs as a second active agent in a combination therapy. In an exemplary form, VLPs including two cancer antigens and optionally encapsidating a TLR agonist are delivered to a subject in need thereof together with an encapsidated or unencapsidated checkpoint inhibitor, or an encapsidated or unencapsidated STING agonist, or together with an encapsidated or unencapsidated checkpoint inhibitor and an encapsidated or unencapsidated STING agonist.

The Examples illustrate that the combination therapies are effective to treat and prevent cancer in a subject. Therefore, in some embodiments, the combination therapy performs similarly, or better than the individual components when used alone. In some forms, the combination provides improved efficacy in treating cancer as compared to the individual components alone. In some forms, although the cancer killing effect of the combination is similar to the individual components, the duration of efficacy of the treatment is longer because the cancer does not become resistant to the treatment. This allows the combination therapies to be administered in combination with or as an alternative to other chemotherapy, a first line therapy, or a second line or subsequent therapy.

A treatment regimen of the combination therapy can include one or multiple administrations of VLPs with one or more additional chemotherapeutic agent. A treatment regimen of the combination therapy can include one or multiple administrations of a checkpoint inhibitor, alone or in combination with a STING agonist. In certain embodiments, a checkpoint inhibitor, alone or in combination with a STING agonist can be administered simultaneously with a dose of VLPs. Where a checkpoint inhibitor, alone or in combination with a STING agonist are administered at the same time as the VLPs, the VLPs can be in the same pharmaceutical composition.

In some embodiments a checkpoint inhibitor, alone or in combination with a STING agonist are administered sequentially with the VLPs, for example, in two or more different pharmaceutical compositions. In certain embodiments, the checkpoint inhibitor, alone or in combination with a STING agonist is administered prior to the first administration of the VLPs. In other embodiments, the VLPs are administered prior to the first administration of the checkpoint inhibitor, alone or in combination with a STING agonist. For example, the checkpoint inhibitor, alone or in combination with a STING agonist can be administered to a subject on the same day as the VLPs. Alternatively, the checkpoint inhibitor, alone or in combination with a STING agonist and the VLPs are administered to the subject on different days.

The VLPs can be administered at least 1, 2, 3, 5, 10, 15, 20, 24 or 30 hours or days prior to or after administering of the one or more additional chemotherapeutic agents. Alternatively, the one or more additional chemotherapeutic agents can be administered at least 1, 2, 3, 5, 10, 15, 20, 24 or 30 hours or days prior to or after administering of the VLPs. In certain embodiments, additive or more than additive effects of the administration of one or more additional chemotherapeutic agents in combination with one or more VLPs is evident after one day, two days, three days, four days, five days, six days, one week, or more than one week following administration.

Dosage regimens or cycles of the agents can be completely or partially overlapping, or can be sequential. For example, in some embodiments, all such administration(s) of the one or more additional chemotherapeutic agents occur before or after administration of the VLPs. Alternatively, administration of one or more doses of the VLPs can be temporally staggered with the administration of one or more additional chemotherapeutic agents to form a uniform or non-uniform course of treatment whereby one or more doses of one or more additional chemotherapeutic agents are administered, followed by one or more doses of VLPs, followed by one or more doses of one or more additional chemotherapeutic agents; or one or more doses of VLPs are administered, followed by one or more doses of one or more additional chemotherapeutic agents, followed by one or more doses of VLPs; etc., all according to whatever schedule is selected or desired by the researcher or clinician administering the therapy.

An effective amount of each of the agents can be administered as a single unit dosage (e.g., as dosage unit), or sub-therapeutic doses that are administered over a finite time interval. Such unit doses may be administered on a daily basis for a finite time period, such as up to 3 days, or up to 5 days, or up to 7 days, or up to 10 days, or up to 15 days or up to 20 days or up to 25 days, are all specifically contemplated.

### D. Dosages and Effective Amounts

In some in vivo approaches, the compositions of VLPs are administered to a subject in a therapeutically effective amount. The term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease or disorder, and the treatment being effected.

For all of the compounds described, as further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired.

Generally dosage levels of between 0.001 and 100 mg/kg of body weight daily are administered to subjects such as mammals, most preferably, humans. Generally, for intravenous injection or infusion, dosage may be lower.

For example, VLPs can be in an amount effective to deliver antigen to a subject and induce the proliferation and clonal expansion of B cells, T cells or induce the migratory or chemotactic activity of macrophages. Therefore, In some forms, the VLPs (optionally including encapsulated agents) are in an amount effective to stimulate a primary immune response to an antigen in a subject. In a preferred form the effective amount of VLPs does not induce significant cytotoxicity in the cells of a subject compared to an untreated control subject. Preferably, the amount of VLPs is effective to prevent or reduce the infection or onset of a disease or disorder in a subject compared to an untreated control.

In a particular form, VLPs are in an amount effective to induce presentation of an antigen by antigen-presenting cells. For example, VLPs can be in an amount effective to induce T cell activation in response to an exogenous polypeptide delivered to the APCs of a subject by the VLPs. In a further form, the one or more VLPs are in an amount effective to decrease the amount of antigen required to stimulate a robust or protective immune response to the antigen in a subject. The VLPs can be effective to induce the production or antibodies to an antigen encoded by the VLPs.

Thus, the VLPs can be effective to enhance the amount of antigen-specific immune cells in a subject. For example the amount of antigen-specific immune cells in a subject can be increased relative to the amount in an untreated control. For example, VLPs can be effective to induce several signaling pathways controlling cellular immune activities, including cellular proliferation, chemotaxis and actin reorganization. Preferably the effective amount of VLPs does not cause cytotoxicity. The effective amount of VLPs to provide adaptive immunity to an encoded antigen or allergen should not generate a significant systemic increase in inflammatory cytokine production, including IFN.

In some forms, VLP can be used to immunize a subject against a cancer, an infectious disease or an allergen using only a single dose. Therefore, in some forms, only a single administration is required with no boosting. In other forms, enhanced or prolonged immunity, such as protective immunity, is achieved when one or more additional doses are used to boost the immune response to a first or previous administration.

### E. Controls

The effect of VLPs can be compared to a control. Suitable controls are known in the art and include, for example, untreated cells or an untreated subject. In some forms, the control is untreated tissue from the subject that is treated, or from an untreated subject. Preferably the cells or tissue of the control are derived from the same tissue as the treated cells or tissue. In some forms, an untreated control subject suffers from, or is at risk from the same disease or condition as the treated subject. For example, in some forms, an untreated control subject does not raise an immune response to the antigen(s).

The disclosed compositions and methods can be further understood through the following numbered paragraphs.

1. A composition for antigen-specific activation of antigen presenting cells (APC), optionally dendritic cells, including
(a) synthetic Virus Like Particles (VLPs);
(b) one or more species of a binding agent(s) that targets APCs, optionally wherein the binding agent targets and/or binds to one or more C-type lectin receptors (CLRs) optionally selected from DC-SIGN, Dectin and MGL, optionally wherein the binding agent is a DC-SIGN ligand(s); and
(c) two or more species of peptide antigen(s),
wherein the binding agent(s) and the antigen(s) are displayed at the outer surface of the synthetic VLP.

2. The composition of paragraph 1, wherein the composition includes two or more species of VLPs,
wherein each species of VLP includes a single species of peptide antigen displayed at the outer surface of the VLP.

3. The composition of paragraph 1, wherein the composition includes one or more species of VLPs,
wherein each species of VLP includes two or more species of peptide antigen(s) displayed at the outer surface of the VLP.

4. The composition of any one of paragraphs 1 to 3, wherein each VLP includes at least one peptide antigen species attached to at least one viral capsid protein within the VLP.

5. The composition of any one of paragraphs 1 to 4, wherein the two peptide antigen species are attached to a single viral capsid protein with a VLP.

6. The composition of any one of paragraphs 1 to 4, wherein the two peptide antigen species are each independently attached to two different viral capsid proteins within the same VLP.

7. The composition of any one of paragraphs 1 to 6, wherein one or both of the peptide antigen species is attached to the viral capsid protein via a polypeptide linker.

8. The composition of paragraph 7, wherein the linker polypeptide is a protease cleavable linker polypeptide including SEQ ID NO:7.

9. The composition of any one of paragraphs 1 to 8, wherein the VLP includes the Leviviridae PP7 capsid protein.

10. The composition of any one of paragraphs 1 to 9, wherein the VLP includes the capsid protein of SEQ ID NO:1 or SEQ ID NO:3.

11. The composition of any one of paragraphs 1 to10, wherein the binding agent is a DC-SIGN ligand(s) including mannose or fucose.

12. The composition of any one of paragraphs 1 to 11, wherein the binding agent is a DC-SIGN ligand(s) including a phenyl-mannose.

13. The composition of any one of paragraphs 1 to 11, wherein the binding agent is a DC-SIGN ligand(s) including aryl-mannose, aryl-fucose, or combinations thereof, and optionally is the ligand of Formula I.

14. The composition of any one of paragraphs 1 to 13, further including an immunostimulatory agent.

15. The composition of paragraph 14, wherein the immunostimulatory agent is a Toll-like receptor (TLR) agonist.

16. The composition of paragraph 15, wherein the TLR agonist is a nucleic acid, preferably a microbial nucleic acid, more preferably a bacterial nucleic acid.

17. The composition of paragraph 15 or 16, wherein the TLR agonist is encapsidated within the VLP.

18. The composition of any one of paragraphs 1 to 17, wherein at least one species of the peptide antigen includes an MHC class I epitope, or an MHC class II epitope.

19. The composition of any one of paragraphs 1 to 18, wherein the two or more species of antigen includes an MHC class I epitope and an MHC class II epitope.

20. The composition of any one of paragraphs 1 to 19, wherein at least one or the peptide antigen species is a tumor associated antigen (TAA), preferably wherein the two species of peptide antigen include two different tumor associated antigens.

21. The composition of paragraph 20, wherein the one or more tumor associated antigen(s) are individually selected from the group including an oncogene expression product, an alternatively spliced protein, a mutated gene product, an over-expressed gene product, an aberrantly expressed gene product, an antigen produced by an oncogenic virus, an oncofetal antigen, a protein with altered cell surface glycolipids, and combinations thereof.

22. The composition of paragraph 20 or 21, wherein the at least one tumor antigen is a tumor specific antigen associated with a cancer selected from the group including bladder, brain, breast, cervical, colo-rectal, esophageal, kidney, liver, lung, naso-pharangeal, pancreatic, prostate, skin, stomach, uterine, ovarian, testicular and hematologic cancer.

23. The composition of paragraph 20 or 21 or 22, wherein at least one antigen is a personalized neoantigen isolated from a subject, preferably wherein the two species of peptide antigen includes two different personalized neoantigens isolated from a subject.

24. The composition of any one of paragraphs 1 to 23, wherein at least one peptide antigen is derived from, or raises an immune response to a virus, a bacterium, a fungi, a protozoan, a nematode, a plant, or an insect.

25. The composition of paragraph 24, wherein the antigen is derived from a pathogenic virus.

26. The composition of paragraph 25, wherein the viral antigen is derived from a virus selected from the group including a Coronavirus, Influenza virus, Ebola virus, Zika Virus, and combinations thereof.

27. The composition of any one of paragraphs 1 to 26, wherein the VLPs include one or more additional active agents, wherein the additional active agents are encapsulated within, or displayed upon the surface of the VLPs.

28. The composition of paragraph 27, wherein the VLPs encapsulate one or more additional active agents selected from the group including a therapeutic agent, a prophylactic agent, a diagnostic agent, and an adjuvant.

29. A pharmaceutical composition including the composition of any one of paragraphs 1 to 28, and a pharmaceutically acceptable excipient for administration to a subject in vivo.

30. The pharmaceutical composition of paragraph 29, further including one or more additional active agents, wherein the additional active agents are not encapsulated within, or displayed upon the surface of the VLPs.

31. The pharmaceutical composition of paragraph 30, wherein the active agent is selected from the group including a therapeutic agent, a prophylactic agent, a diagnostic agent, and an adjuvant.

32. The pharmaceutical composition of paragraph 30 or 31, wherein the active agent is a chemotherapeutic agent.

33. The pharmaceutical composition of paragraph 32, wherein the active agent is selected from the group including a checkpoint inhibitor and a STING agonist, or both a checkpoint inhibitor and a STING agonist.

34. A vaccine including the composition of any one of paragraphs 1 to 29, or the pharmaceutical composition of any one of paragraphs 30 to 33 in an amount effective to activate dendritic cells and stimulate an immune response to the antigen in a subject, optionally further including an adjuvant.

35. A method of generating an immune response to an antigen in a subject, including administering the composition of any one of paragraphs 1 to 29, or the pharmaceutical composition of any one of paragraphs 30 to 33, or the vaccine of paragraph 34 to the subject in an amount effective to activate dendritic cells and stimulate an immune response to the antigen in the subject.

36. The method of paragraph 35, wherein the subject has, or is at risk of having an infectious disease, wherein the antigen is derived from or stimulates an immune response to a pathogen associated with the disease, and wherein the antigen stimulates an immune response to the pathogen in the subject.

37. The method of paragraph 36, wherein the subject has, or is at risk of having cancer, wherein the antigen is a tumor antigen, and wherein the antigen stimulates an immune response to the tumor antigen in the subject.

38. The method of paragraph 37, wherein the cancer is selected from the group including bladder, brain, breast, cervical, colo-rectal, esophageal, kidney, liver, lung, nasopharangeal, pancreatic, prostate, skin, stomach, uterine, ovarian, testicular and hematologic cancer.

39. The method of any one of paragraphs 35 to 38, wherein the immune response is a T-helper 1 (TH1)-type immune response to the antigen.

40. The method of any one of paragraphs 35 to 39, further including administering one or more additional active agents to the subject.

41. The method of paragraph 40, wherein the active agent is selected from the group including a therapeutic agent, a prophylactic agent, a diagnostic agent, and an adjuvant.

42. The method of paragraph 41, wherein the active agent is a chemotherapeutic agent.

43. The method of paragraph 42, wherein the active agent is selected from the group including a checkpoint inhibitor and a STING agonist, or both a checkpoint inhibitor and a STING agonist.

44. The method of paragraph 43, wherein the checkpoint inhibitor is a PD-1 inhibitor.

45. The method of paragraph 42 or 43, wherein the PD-1 inhibitor is an anti-PD1 antibody.

46. The method of paragraph 43 or 45, wherein the STING agonist is a 2/3/-cGAMP.

47. The method of any one of paragraphs 40 to 46, wherein administering the additional active agent to the subject together with the composition of VLPs provides more effective immunity to the antigen than the response raised to the antigen when the additional active agent or the composition of VLPs are administered to the subject alone.

48. The VLP of any one of paragraphs 1 to 29.

49. A method of synthesizing a DC-SIGN ligand, including the synthesis scheme set forth in FIG. 1B.

50. A method of synthesizing an aryl-bearing core displaying monosaccharides or oligosaccharides or glycans, including the synthesis scheme set forth in FIG. 1B modified by substituting the mannopyranose with the alternative monosaccharide(s) or oligosaccharide(s) or glycan(s).

### EXAMPLES

Vaccines have completely transformed infectious disease burden; however, the development of vaccines against cancer has been largely unsuccessful. Traditional vaccines rely on the induction of humoral (Th2) immunity, which consists of neutralizing antibodies against exogenous pathogens. Therapeutic cancer vaccines, however, require induction of cellular (Th1) immunity—helper (CD4*) and cytotoxic (CD8*) T cells that can recognize and destroy cancer cells. Priming the immune system against cancer is challenging because cancer cells often employ immune evasion strategies, such as overexpression of inhibitory cell-surface glycans and secretion of anti-inflammatory cytokines, to prevent anti-tumor immunity (Kim, et al., Cancer Research 66, 5527-5536 (2006)). In order to overcome the immunosuppressive tumor microenvironment (TME), it is necessary to induce and recruit robust, tumor-specific immune cells to the tumor site. To initiate adaptive immunity against external pathogens, the immune system's central player is dendritic cells (DCs) (Rossi & Young, The Journal of Immunology 175, 1373-1381 (2005)). As professional antigen-presenting cells, DCs internalize pathogens, including viruses, parasites, and fungi, and present processed epitopes on major histocompatibility complex (MHC) molecules for T cell priming (Guermonprez, et al., Annu. Rev. Immunol 20, 621-667 (2002)). Additionally, in response to pathogen engagement, DCs secrete specific cytokines that activate Th1, Th2, or tolerogenic (Treg) immunity (Kapsenberg, Nature Reviews Immunology 3, 984-993 (2003)). Due to their key role in modulating immunity via T cell polarization, modulating DC responses is important for inducing tumor antigen-specific, Th1-type immunity. Induction of Th1-type cellular immunity requires upregulation of DC co-stimulatory molecules and secretion of pro-inflammatory cytokines (Salerno-Gonçalves& Sztein, Trends in Microbiology 14, 536-542 (2006)). Due to the complexity of DC machinery, methods to effectively direct DC behavior remain limited. The disclosure herein provides therapeutic cancer vaccines that can leverages combinatorial engagement of specific pattern recognition receptors (PRRs)—both lectins and toll-like receptors (TLRs)—to induce Th1-type DC signaling and tumor antigen-specific T cells in vivo.

PRRs have emerged as promising targets for DC-based immunotherapies (Wculek, et al., Nature Reviews Immunology 20, 7-24 (2020)). Lectins are a family of PRRs that are present on the cell surface of DCs. These carbohydrate-binding proteins play a fundamental role in pathogen recognition and immune system modulation (van Kooyk & Rabinovich, Nature Immunology 9, 593-601 (2008)). The native role of DC lectins is to recognize and bind unique glycan displays on the surface of pathogens, facilitating highly efficient internalization of antigens and subsequent modulation of the immune response. Among them, the lectin DC-SIGN (dendritic cell-specific intercellular adhesion molecule-3-grabbing nonintegrin or CD209) acts by binding mannose and fucose oligosaccharides on numerous exogenous pathogens, leading to distinct (Th1, Th2, or Treg) immune responses (Geijtenbeek & Gringhuis, Nature Reviews Immunology 9, 465-479 (2009), Valverde, et al., Chem Bio Chem 21, 2999-3025 (2020)). Another class of PRRs of increasing interest for vaccine adjuvants is TLRs. TLRs recognize various pathogen-associated molecular patterns, resulting in the activation of signaling pathways that upregulate cytokines, chemokines and costimulatory molecules (Dowling & Mansell, Clin Transl Immunology 5, e85 (2016). TLR7 specifically detects infection by binding bacterial or viral single-stranded RNA (ssRNA), evoking pro-inflammatory cytokine secretion (Diebold, et al., Science 303, 1529-1531 (2004)). The activation of TLR7 on DCs improves antigen cross-presentation via increased formation of MHC-peptide complexes, up-regulation of costimulatory molecules (CD80, CD86, CD40), and release of LL-12 (Larange, et al., J Leukoc Biol 85, 673-683 (2009)). There is increasing evidence that combinatorial engagement of lectins and TLRs can direct DC-mediated immunity (Son, et al., Nat Biomed Eng 7, 72-84 (2023), van Haren, et al., J Immunol 197, 4413-4424 (2016)). While antigen recognition by lectins alone may induce regulatory DC responses, the contribution of TLR signaling can switch a phenotype from regulatory to immunostimulatory (van Vliet, et al., Immunol Cell Biol 86, 580-587 (2008)).

A synthetic aryl mannoside ligand for DC-SIGN, mimicking the HIV-1 gp120 envelope protein, that facilitated immune evasion has been reported (Jarvis, et al., Proc Natl Acad Sci USA 116, 14862-14867 (2019)). Engagement of DC-SIGN and TLR7 by glycosylated VLPs results in pro-inflammatory, Th1-type signaling (Alam, et al., ACS Nano 15, 309-321 (2021)). VLPs have emerged as versatile platforms for antigen delivery due to their innate immunogenicity, their ability to drain to lymph nodes, and their versatility for antigen display (Nooraei, et al., Journal of Nanobiotechnology 19, 59-59 (2021)). VLPs are non-replicating nanostructures composed of self-assembling coat protein monomers. Encapsulation of single-stranded RNAs (ssRNAs) from the expression host enable binding of toll-like receptors (TLRs) that results in immune activation (Tumban, et al., Vaccine 31, 4647-4654 (2013)).

Given that Th1-type immunity is important for therapeutic cancer vaccines, experiments were designed to determine if immunostimulatory glycan-lectin interactions could be leveraged to redirect anti-cancer immune responses. As a result cancer vaccines that mimicked exogenous pathogens via simultaneous presentation of lectin and TLR ligands with tumor antigens were developed. As illustrated in the experiments below, targeting the endocytic lectin DC-SIGN facilitated highly efficient tumor antigen internalization by DCs. Furthermore, glycosylated VLPs elicited superior DC activation and induce more robust tumor-specific T cells compared to non-targeted VLPs. Treatment with DC-targeted VLPs resulted in enhanced tumor growth inhibition and prolonged survival compared to non-targeted VLPs in a challenging mouse melanoma model. Targeting the immunostimulatory lectin DC-SIGN in conjugation with TLR7 was therefore shown to be an effective, self-adjuvanting method for enhancing anti-tumor cellular immune responses. Thus DC-activating VLP platforms for in vivo T cell priming, and uses thereof, most particularly in the field of cancer immunotherapies, are provided.

### Preparation and Functionalization of VLPs

Cancer immunotherapies primarily rely on CD8+ T cells to target tumors; however, it has recently become clear that CD4⁺ T cell help enhances CD8⁺ T cell expansion and function (Borst, et al., Nature Reviews Immunology 18, 635-647 (2018)). To best elicit CD4⁺ and CD8⁺ T cell activation in a cancer immunotherapeutic, it was desired to engineer VLPs that could induce DC programming of both such T cells. Heavily mannosylated Qβ VLPs bearing genetically encoded OvaII (Ova323-339) peptides could induce an antigen-specific Th1 response in vivo (Alam, et al., ACS Nano 15, 309-321 (2021)). Analogous Qβ VLPs bearing a similar density of aryl mannose ligands and genetically encoded OvaI (Ova257-264) peptides were not easily accessible, since the OvaI peptide has an internal lysine residue that is likely to be acylated in the course of particle conjugation. Attempts to chemically derivatize Qβ VLPs with high densities of glycans and peptides (>540 Man and >40 peptides per particle) were not successful, since these particles were prone to irreversible aggregation.

In order to maximize the loading of peptide antigens on VLPs, the use of the PP7-PP7 platform was utilized. This platform has a higher density of accessible amines for functionalization (˜1200 per particle), and a higher tolerance to incorporate encoded peptides (Zhao, et al., ACS Nano 13, 4443-4454 (2019)). To facilitate particle loading with diverse peptide antigens, including peptides containing nucleophilic amino acids (e.g. lysine, cysteine), a two-stage click chemistry strategy was utilized. Amine-reactive esters were used to install a low density of fluorescent dyes and a high density of azides on VLPs. Following particle purification, the intermediate particles could then be coupled to alkyne-containing glycomimetic ligands (Man, PE) and/or peptide antigens (OTI, OTII) via ligand-accelerated copper(I)-catalyzed azide-alkyne cycloaddition (CuAAC).

The extent of mannose and peptide functionalization on each particle was controlled by sequentially mixing in the peptide-alkynes (less reactive, fewer copies desired per particle) and mannose or PE-alkynes (more reactive, maximal number of copies desired per particle). The resulting conjugates were analyzed by LC-ESI-TOF-MS to determine the average loading density of each functional unit, and by DLS and FPLC to confirm particle integrity. Thus, particles PP7-PE₁₀₀₀ (PE), PP7-PE₉₀₀-OvaI₁₀₀ (PE-OvaI), PP7-PE₉₀₀-OvaII₁₀₀ (PE-OvaII), PP7-Man₁₀₀₀ (Man), PP7-Man₉₀₀-OvaI₁₀₀ (Man-OvaI), and PP7-Man₉₀₀-OvaII₁₀₀ (Man-OvaII) were generated. Particles constructed in this manner take advantage of innate immunogenic properties and are highly programmable to achieve antigen-selective immunity. DC activation is enhanced by costimulatory signaling via dual mannose-DC-SIGN and ssRNA-TLR7 interactions. Tumor antigens can be attached by a click chemistry approach and released by endosomal proteolytic processing to facilitate loading on MHC molecules.

### Engineered VLPs Engage DC-SIGN and TLR7 for Efficient Uptake and DC Activation

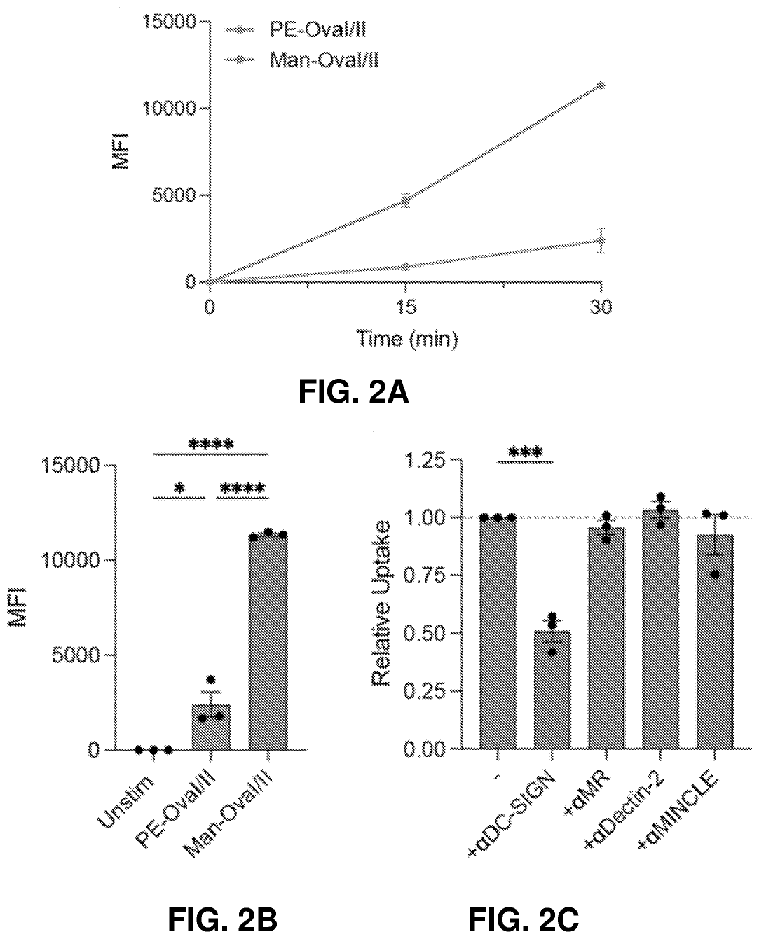

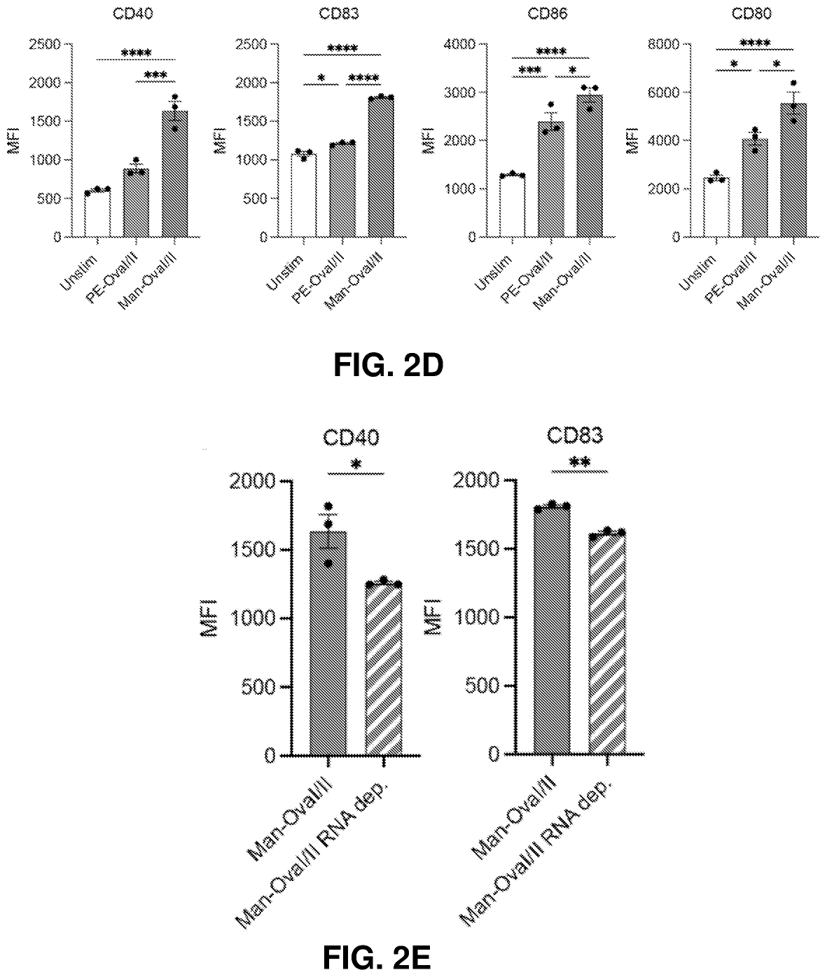

Efficient T cell priming needs efficient antigen uptake and DC activation. DC endocytosis and downstream signaling occur upon PRR recognition of pathogens. Thus, VLP Man-OvaI/II were engineered to deliver tumor antigens in a pro-inflammatory context. To investigate the efficiency of particle uptake and DC activation as mediated by DC-SIGN, the uptake of Man-OvaI/II was compared to a control particle, PE-OvaI/II. Fluorophore-labeled VLPs were incubated with monocyte-derived dendritic cells (moDCs) over the course of 30 min. Uptake of Man-OvaI/II was significantly higher (5-fold increase) than PE-OvaI/II (FIG. 2A, 2B). The same trend was observed with the corresponding particles without Ova antigens, showing that VLP functionalization with Ova peptides did not interfere with the glycan-lectin interactions. To investigate the endocytic role of DC-SIGN over other mannose-binding lectins, moDCs were pre-treated with blocking antibodies against DC-SIGN, mannose receptor (MR or CD206), Dectin-2, or Mincle for 20 min followed by incubation with VLPs. Pre-treatment with DC-SIGN—but not MR, Dectin-2, or Mincle—resulted in a significant decrease in Man-OvaI/II uptake, confirming that DC-SIGN is the major endocytic PRR for Man-OvaI/II (FIG. 2C). Uptake of either PE or PE-OvaI/II was not significantly diminished upon pre-treatment with any of the blocking antibodies, indicating that any uptake of these particles is likely mediated by non-specific mechanisms such as phagocytosis. These data indicate that Man-OvaI/II uptake occurs mostly by DC-SIGN-mediated endocytosis.

Moreover, experiments were designed to determine if the endocytic ability of DC-SIGN would facilitate the delivery of VLP-encapsulated ssRNAs to the endosomal receptor TLR7. The VLP protein shell is degraded within the endosomal compartments, releasing the ssRNAs for stimulation of local TLR7 receptors (Mohsen, et al., Seminars in Immunology 34, 123-132 (2017)). To assess co-engagement of DC-SIGN and TLR7, confocal microscopy was performed on moDCs stimulated with VLPs and labeled with anti-DC-SIGN and anti-TLR7 antibodies. Confocal images revealed that Man-OvaI/II was efficiently internalized by moDCs within 30 min whereas PE-OvaI/II was present only at low levels in the cells, corroborating the enhanced uptake of mannosylated VLPs as measured by flow cytometry. Additionally, internalization and colocalization of DC-SIGN with VLPs was only observed with Man-OvaI/II. Colocalization of Man-OvaI/II with both TLR7 and DC-SIGN was observed in moDCs after 1 h incubation with Man-OvaI/II. These data further confirm DC-SIGN-mediated uptake of Man-OvaI/II and demonstrate TLR7 engagement by VLPs.

To assess DC activation by VLPs, moDCs were incubated with Man-OvaI/II or PE-OvaI/II for 24 h. Man-OVAI/II induced more robust DC activation compared to PE-OVAI/II, demonstrated by the significant increase in surface expression of activation markers CD40, CD80, CD83, and CD86 (FIG. 2D). To gain insight into the role of TLR7 engagement in DC activation, VLP-Man-OVAI/II particles that were depleted of ssRNA were generated. Notably, Man-OVAI/II induced significantly greater surface expression of DC activation markers CD40 and CD83 as compared to their RNA-depleted counterparts (FIG. 2E). Taken together, mannosylated VLPs target DCs via binding to the cell surface lectin DC-SIGN, facilitating highly efficient endocytosis. Within the endosome, synergistic engagement of DC-SIGN and TLR7 leads to efficient DC activation.

### VLP-Man Elicits Th-1 Type Immune Responses In Vitro

Next, experiments were designed to investigate the signaling pathways, gene expression, and cytokine secretion induced by mannosylated VLP engagement of DC-SIGN and TLR7.

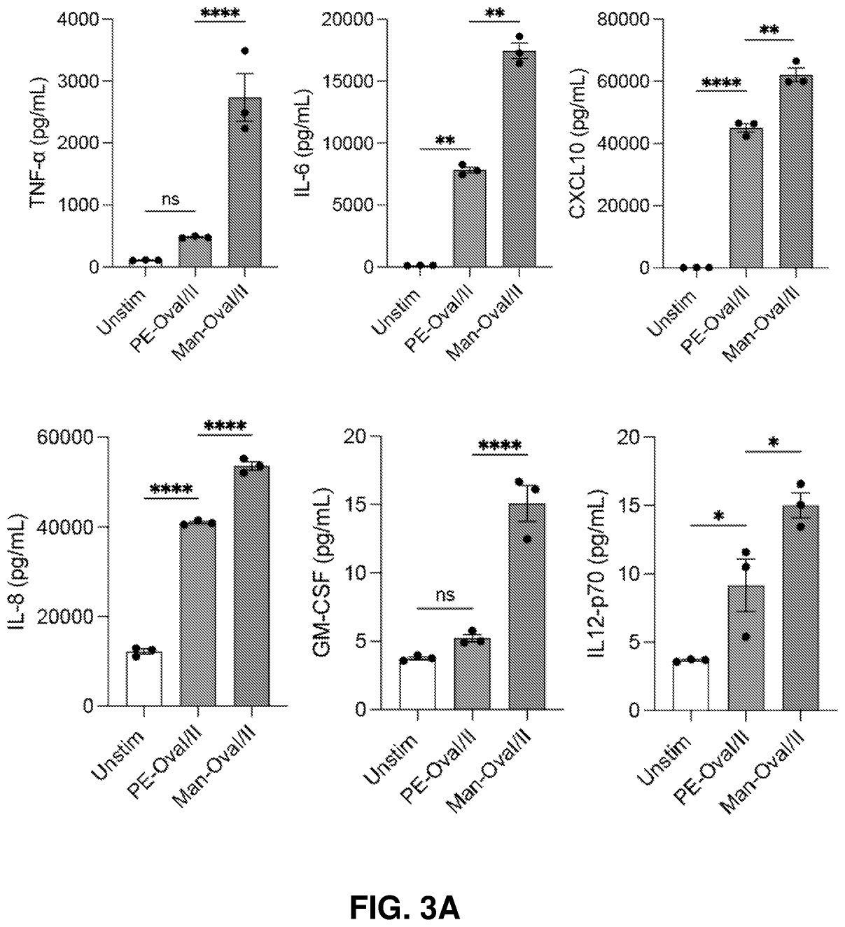

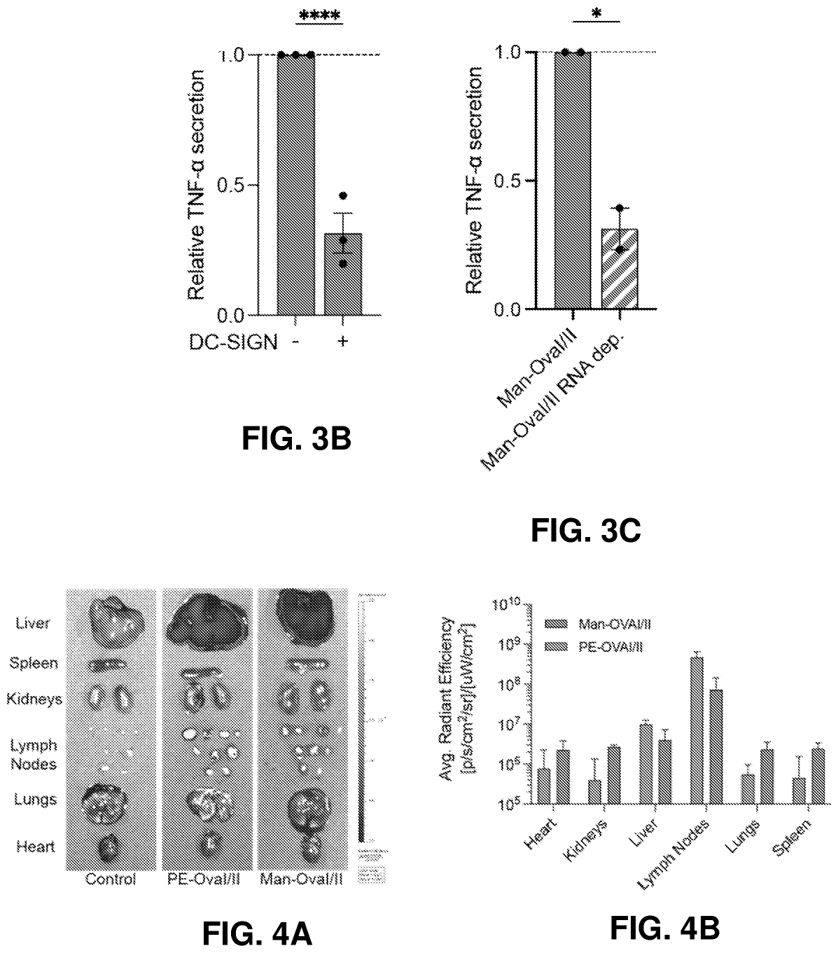

To assess cytokine secretion, moDCs were incubated with VLPs for 32 h and the supernatant was collected for cytokine analysis. moDCs incubated with Man-OvaI/II secreted significantly higher levels of Th1-type, pro-inflammatory cytokines, including TNF-α, IL-6, CXCL10, IL-8, and IFNλ1, as compared to PE-OvaI/II (FIG. 3A). Notably, moDCs pre-treated with blocking antibodies against DC-SIGN showed a significant reduction in TNF-α production following VLP-Man treatment (FIG. 3B). Additionally, RNA-depleted Man-OvaI/II resulted in significantly lower TNF-α production as compared to their counterpart containing encapsulated ssRNAs. This finding shows that engagement of DC-SIGN and TLR7 by Man-OvaI/II triggers a signaling cascade leading to Th1-type, pro-inflammatory cytokine secretion.

### Mannosylated VLPs are Taken Up Preferentially by DCs in Vivo

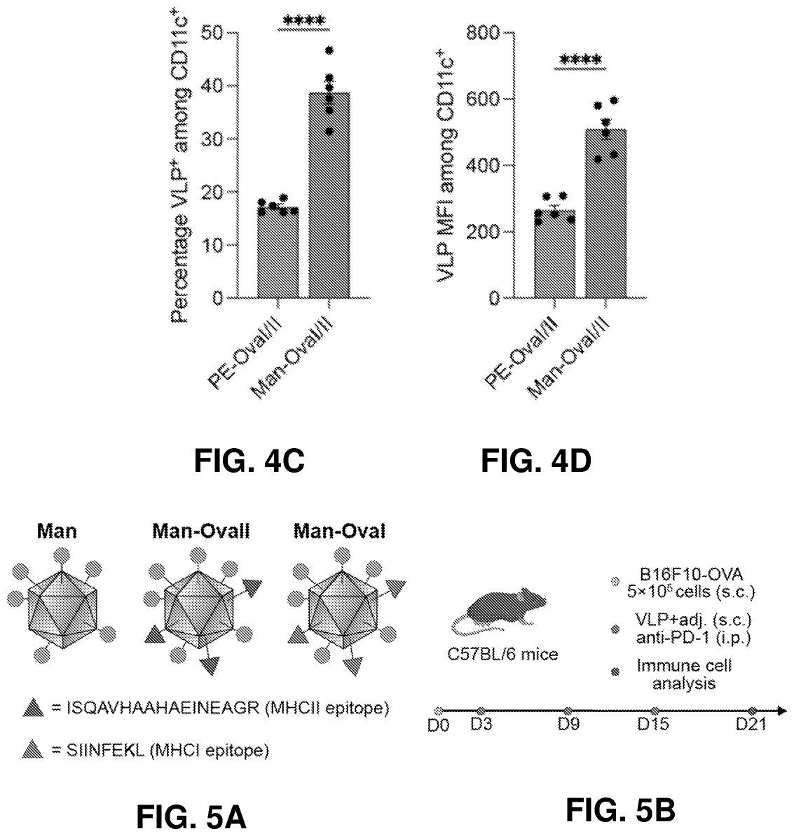

Motivated by the efficient uptake of mannosylated VLPs in moDCs, experiments were designed to test the uptake of VLPs by DCs in vivo. Trafficking of vaccines to the lymph nodes (LNs) is a key step for efficient uptake by DCs and antigen presentation to T cells. Particles on the order of 20-200 nm have been shown to directly reach LNs through lymph drainage within hours of subcutaneous administration (Mohsen, et al., Seminars in Immunology 34, 123-132 (2017)). The size of PP7 VLPs (˜40 nm diameter) is therefore suitable for entering the lymphatic system, rendering them capable of immune priming even when administered at sites distal from both the lymph vessels and the tumor (Mohsen, et al., Seminars in Immunology 34, 123-132 (2017)). To assess the trafficking of the VLPs, C57BL/6J mice were subcutaneously immunized with AF647-labeled VLPs, and fluorescence intensity in the isolated organs was quantified. Selective accumulation of VLPs in the immune cell-rich LNs within 4 hours (FIG. 4A). VLPs were cleared through the liver over the course of 72 hours. To understand whether any anti-tumor activity might occur directly through VLP activity within the tumor, mice inoculated with B16F10-OVA were immunized and VLP fluorescence intensity within the resulting tumor was measured. No localization of the VLPs to the tumor was observed, indicating that any immune responses observed within the tumor are not caused by VLP activity within the tumor, but rather migration of immune cells to the tumor site.

To assess the DC-targeting capacity of VLPs in vivo, VLP uptake by LN-resident DCs was further monitored by flow cytometry. Robust uptake of Man-OvaI/II in DCs was observed by flow cytometry, with a significantly higher percentage of LN DCs being positive for Man-OvaI/II after 12 h as compared PE-OvaI/II (FIG. 4C). Additionally, the MFI of VLPs was higher among DCs of mice immunized with Man-OvaI/II compared to PE-OvaI/II (FIG. 4D). Taken together, mannosylation of the VLPs facilitates higher uptake of VLPs by a larger percentage of DCs in the LNs. Experiments were designed to determine if this increased antigen internalization by DCs will facilitate more robust anti-tumor immunity.

VLPs Induce Antigen-Specific CD4⁺ and CD8⁺ T-Cell Responses In Vivo

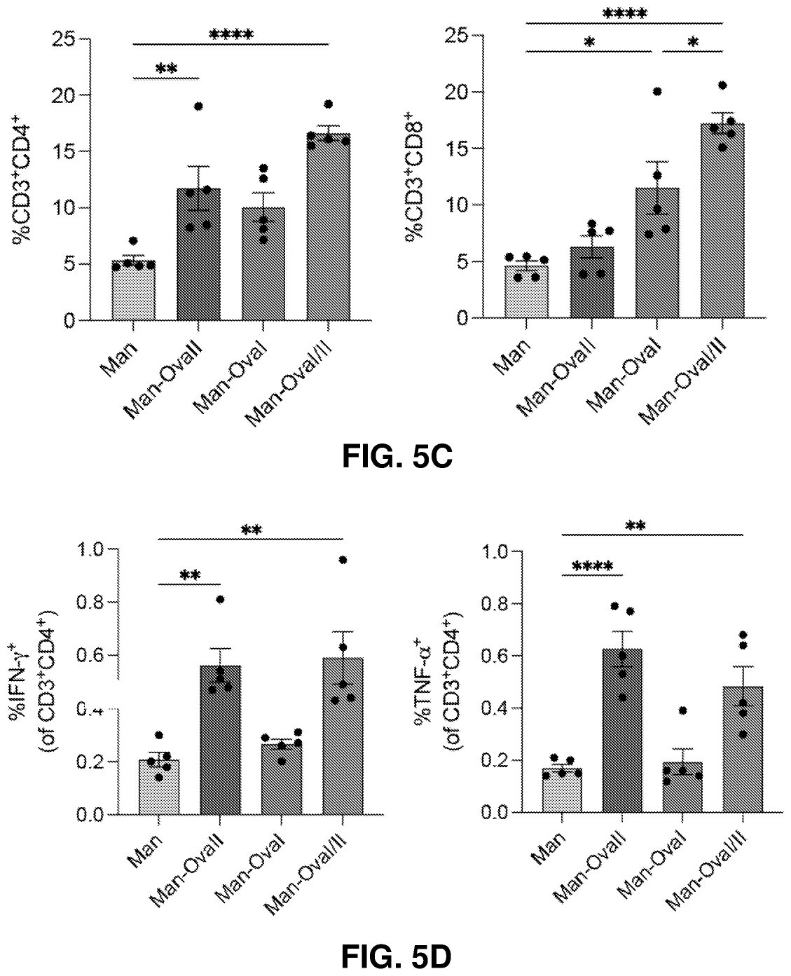

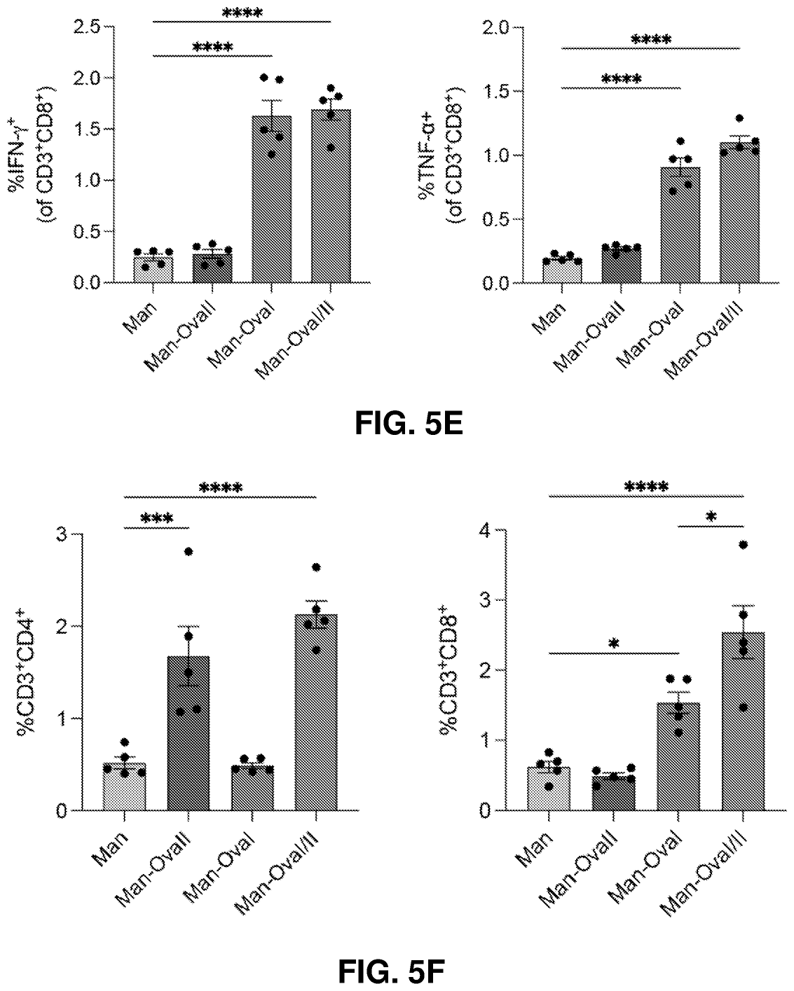

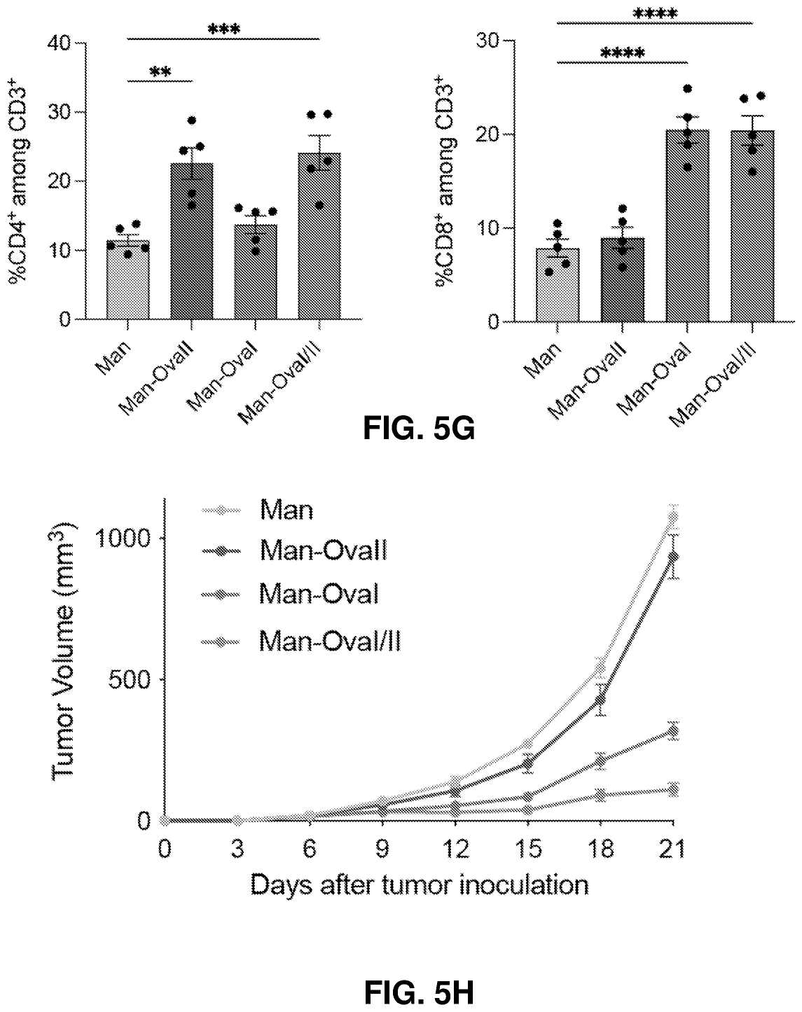

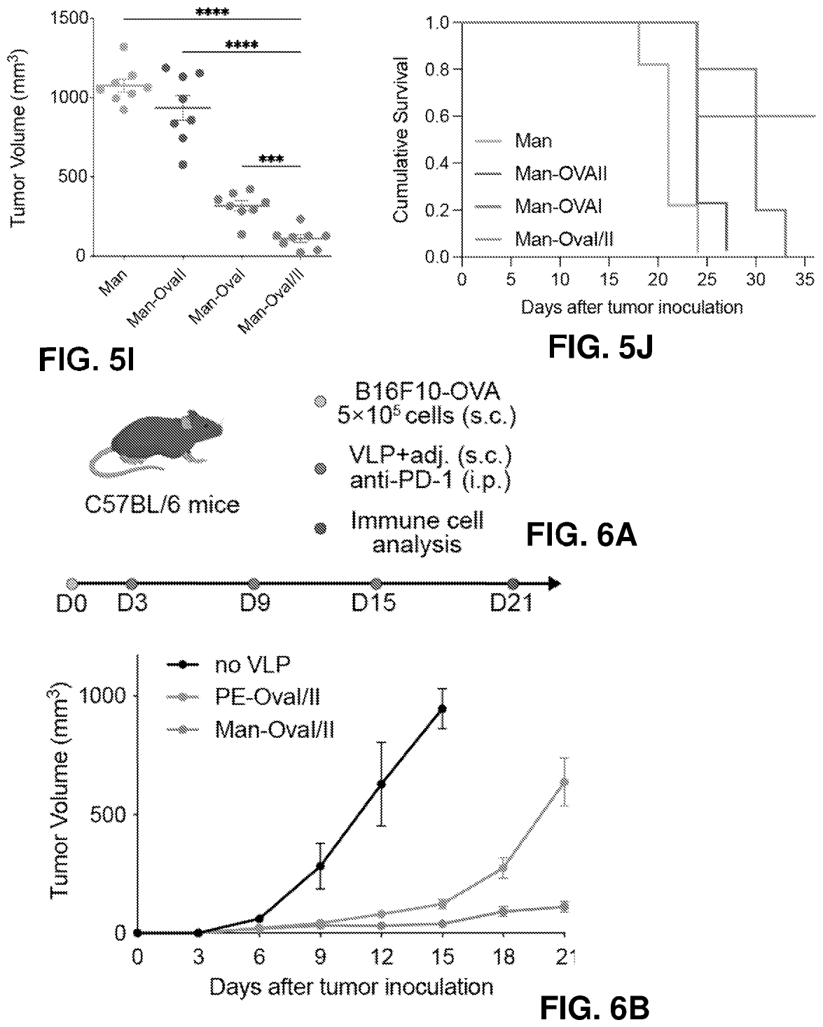

By functionalizing VLPs with both DC-SIGN ligands and tumor antigens, consequences of DC-SIGN engagement on the induction of tumor antigen-specific cellular immune responses could be examined. T cell priming capacity and therapeutic efficacy of the VLPs against melanoma were assessed. Melanoma is the most aggressive skin cancer—it is highly resistant to traditional radio and chemotherapies, and it is often metastatic with rapid systemic dissemination (Tas, Journal of Oncology 2012, 1-9 (2012)). To assess the immunomodulatory effects of the VLPs in an immune-competent and syngeneic mouse transplant, the highly aggressive B16F10-OVA murine melanoma was used as a solid tumor model expressing Ova antigens. The ability of Man-OvaI/II to induce tumor antigen-specific CD4⁺ and CD8⁺ T cells toward tumor growth inhibition was investigated first. Six-week-old C57BL/6J mice were inoculated with B16F10-OVA tumor cells, followed by three immunizations (at 6-day intervals) with VLPs, 2′3′-cGAMP, and anti-PD-1 (FIG. 5A). 2′3′-cGAMP is a dinucleotide agonist for STING which was used as an adjuvant to trigger a pro-inflammatory response. Anti-PD-1 was used to block PD-1/PD-L1 T cell/tumor cell interaction that inhibits T lymphocyte proliferation, survival and effector functions so that the cytotoxic capacity of T cells could be measured. On day 21 (6 days following the last dose), spleens and tumors were isolated for immune cell analysis.

First, whether mannosylated VLPs further conjugated with tumor antigens could induce tumor-antigen specific CD4⁺ and CD8⁺ T cells, respectively was assessed. To this end, T cell induction by mannosylated VLPs displaying no tumor antigens (Man), only an MHCII epitope (Man-OvaII), only an MHCI epitope (Man-OvaI), or a combination of both particles (Man-OvaI/II) (FIG. 5A) were compared. Immunization with Man-OvaII increased percentages of CD4⁺ T cells in the spleen two-fold as compared to immunization with Man only (FIG. 5B). Similarly, immunization with Man-OvaI increased percentages of CD8⁺ T cells in the spleen 2.5-fold as compared to immunization with Man only (FIG. 5B). Interestingly, immunization with the combined particles Man-OvaI/II led to a significantly higher percentage of CD8⁺ T cells in the spleen compared to Man-OvaI alone, indicating that CD4⁺ T cell help augments CD8⁺ T cell proliferation.

To assess T cell specificity against the tumor antigens, splenocytes were restimulated with the antigens OVA(323-339) or OVA(257-264) and used intracellular cytokine staining to quantify the percentage of CD4⁺ and CD8⁺ T cells expressing IFN-γ and TNF-α, cytokines characteristic of Th1-type polyfunctional T cells. Upon restimulation with OVA(323-339), a significant (2.7-fold) increase in the percentage of IFN-γ⁺CD4⁺ T cells and (3.7-fold) increase in the percentage of TNF-α⁺CD4⁺ T cells among spleen cells was detected in the Man-OvaII group compared to the Man group upon (FIG. 5C). Notably, a significant 6.6-fold increase in IFN-γ⁺CD8⁺ T cells and 4.7-fold increase in TNF-α⁺CD8⁺ T cells among spleen cells was detected in the Man-OvaI group compared to the Man group upon restimulation with OVA(257-264) (FIG. 5E). Tumor-antigen specific CD4⁺ and CD8⁺ T cells were similarly observed in splenocytes of mice immunized with Man-OvaI/II. Thus, the simultaneous presentation of exogenous glycoligands with tumor antigens on VLPs facilitated the differentiation of naïve helper T cells into tumor antigen-specific Th1 cells in vivo.

Thus, the ability of the induced T cells to migrate to the tumor site was assessed by flow cytometry analysis of the tumor. Consistent with T cell induction in the spleen, an upregulation of CD4⁺ and CD8⁺ T cells was observed among all cells within the tumor corresponding to immunization with Man-OvaII or Man-OvaI compared to immunization with Man alone (FIG. 5F). The same increases in CD4⁺ and CD8⁺ T cells were observed among CD3⁺ T cells in the tumor (FIG. 5G). Interestingly, a corresponding increase in the percentage of CD8⁺ T cells in the tumor for immunization with Man-OvaI/II was observed compared to Man-OvaI, once again indicating that the CD4⁺ T cell help was promoting CD8⁺ T cell activity (FIG. 5F).

To assess the cytotoxic capacity of these T cells, tumor growth was monitored over the course of 3 weeks. Man-OvaII provided no significant tumor growth inhibition compared to Man only, indicating that CD4⁺ T cell help is not sufficient for providing anti-tumor immunity (FIG. 5H, 5I). Man-OvaI provided significant tumor growth inhibition over Man only, demonstrating that the resulting CD8⁺ T cells were able to achieve significant tumor cell killing (FIG. 5H, 5I). Finally, Man-OvaI/II significantly inhibited tumor growth and prolonged animal survival compared to VLP-Man alone, VLP-Man-OvaII alone, and VLP-Man-OvaI alone (FIG. 5I, 5J). The significant enhancement in tumor growth inhibition by Man-OvaI/II compared to Man-OvaI confirms that CD4⁺ T cells support CD8⁺ T cell proliferation, survival, and tumor cell killing. Overall, the results demonstrated tumor antigen-specific T cell responses that were directly measurable in the splenocytes of mice following vaccination with Man-OvaI/II and observed corresponding therapeutic efficacy in a murine melanoma model.

### Mannosylation Amplifies Anti-Tumor Efficacy of VLPs

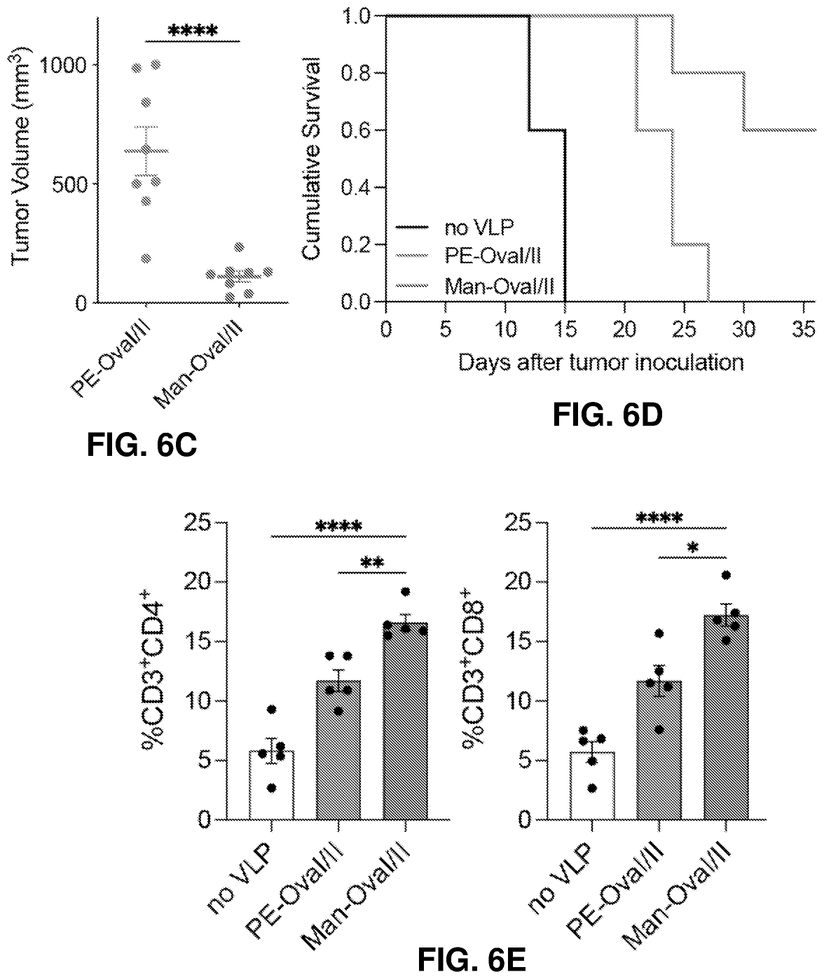

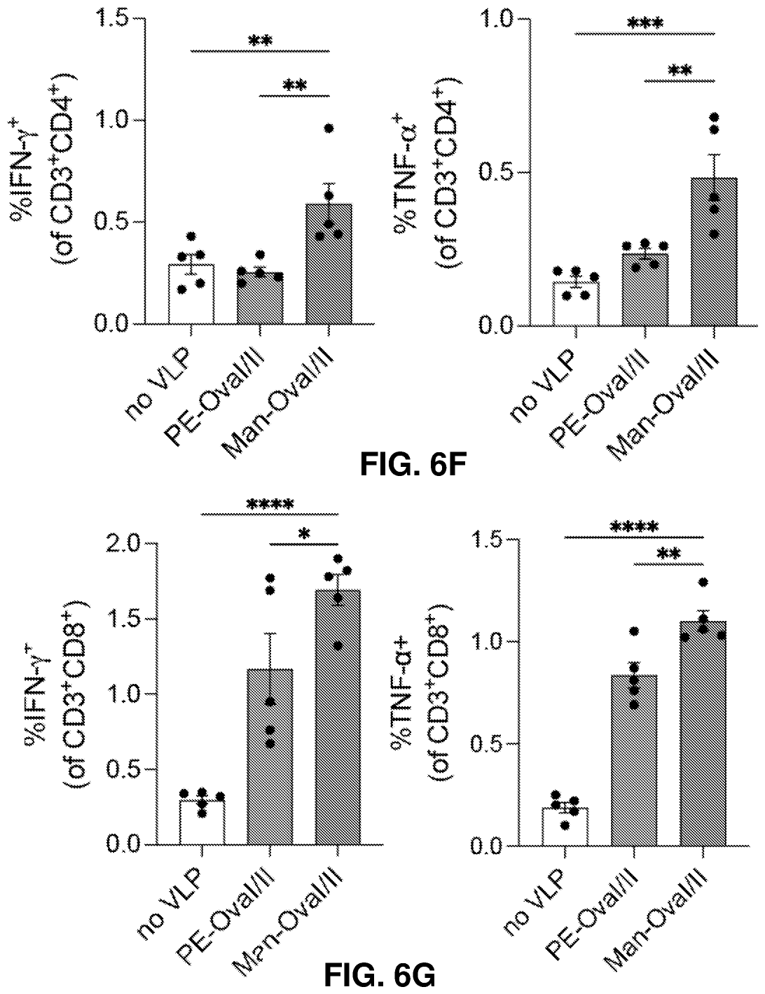

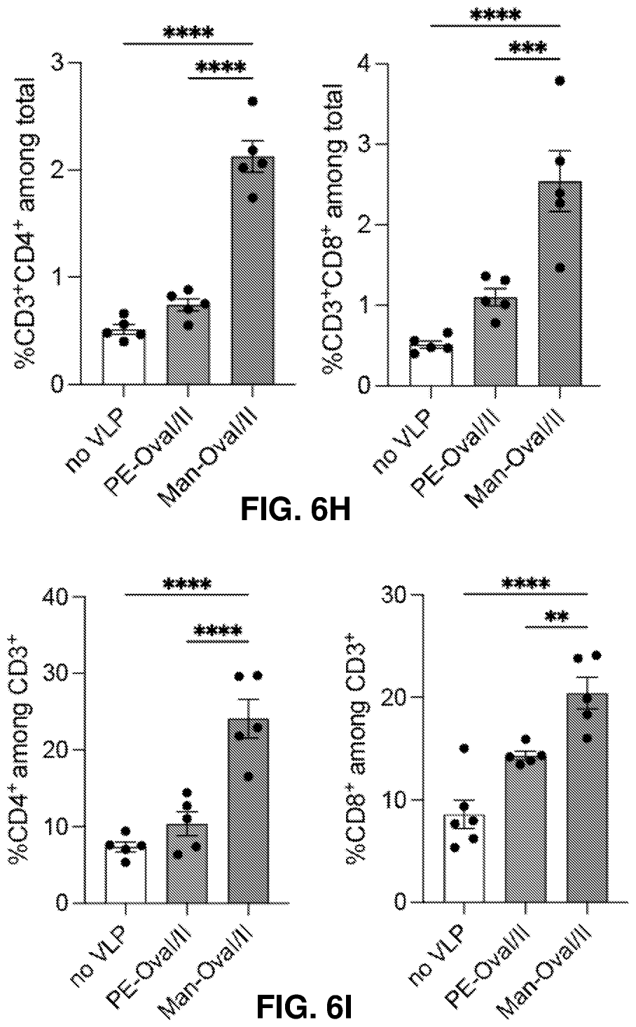

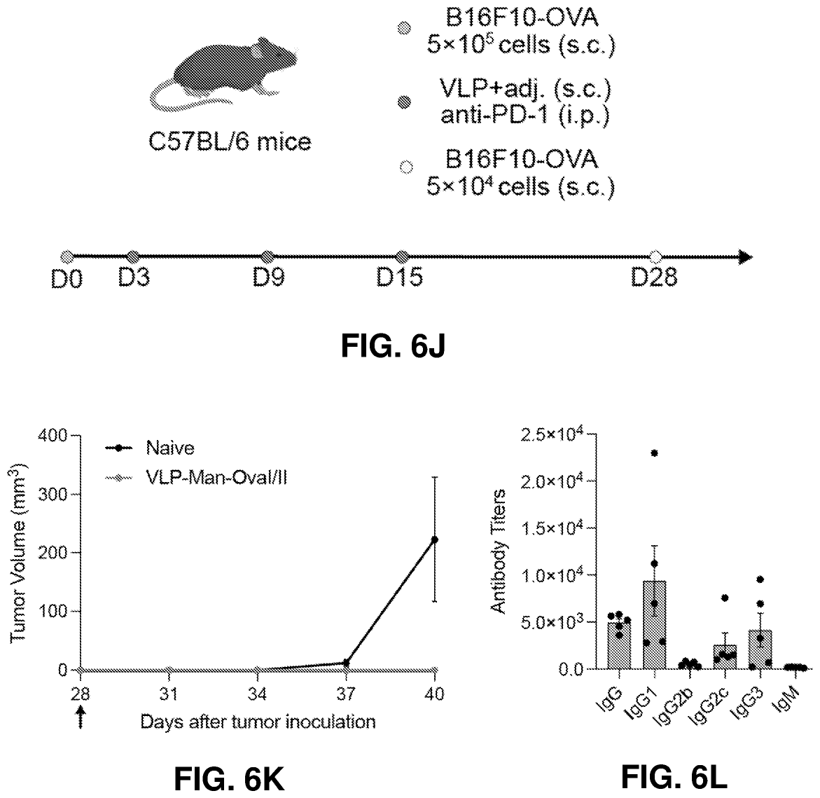

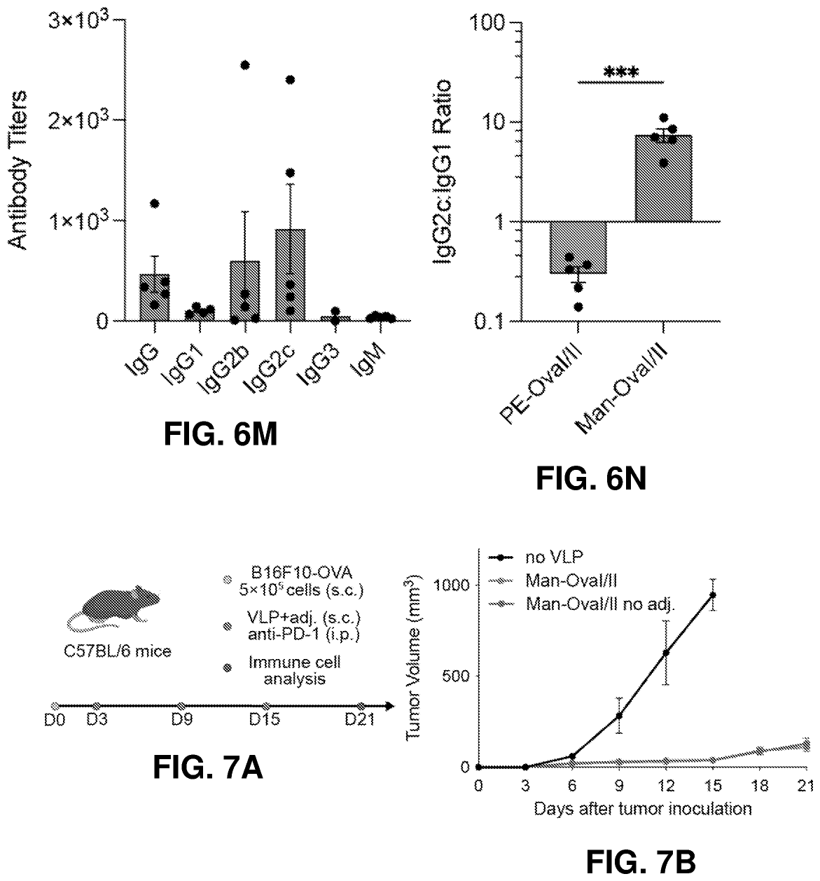

The engagement of DC-SIGN by Man-OvaI/II induced differential DC activation and signaling profile in vitro compared to the nonspecific PE particles. Next, experiments were designed to assess if this superior DC activation translated to a more robust T cell response in vivo. Man-OvaI/II significantly slowed tumor growth and extended survival as compared to PE-OvaI/II (FIG. 6A-6D). The immune landscape was examined on day 21 and it was found that T cell generation paralleled the therapeutic efficacy. Immunization with Man-OvaI/II significantly increased percentages of both CD4⁺ (1.4-fold) and CD8⁺ (1.5-fold) T cells in the spleen as compared to immunization with PE-OvaI/II (FIG. 6E). Not only did the mannosylated particles recruit more T cells to the spleen, but they also induced a higher percentage of tumor antigen-specific T cells compared to the control particles—as measured by IFN-γ and TNF-α secretion upon restimulation with model tumor antigens OVA(323-339) or OVA(257-264). After restimulation with OVA(323-339), mice vaccinated with Man-OvaI/II demonstrated a 2.3-fold increase in the percentage of IFN-γ⁺CD4⁺ T cells and a two-fold increase in the percentage of TNF-α⁺CD4⁺ T cells as compared with mice treated with PE-OvaI/II (FIG. 6F). Analogous results were observed in the CD8⁺ T cell population upon restimulation of the spleen with OVA(257-264)—the percentage of IFN-γ⁺CD8⁺ T cells was 1.5-fold higher and the percentage of TNF-α⁺CD8⁺ T cells was 1.3-fold higher for the Man-OvaI/II cohort compared to the PE-OvaI/II cohort (FIG. 6G). The significant enhancement in tumor-antigen specific CD4⁺ and CD8⁺ T cells for mice immunized with Man-OvaI/II compared to PE-OvaI/II indicates that DC-SIGN engagement drives higher DC activation and antigen presentation. Consistent with the in vitro results, the mannosylated VLPs that more potently activate immune signaling give rise to greater T cell responses.

The extent of immune infiltrate within tumors is emerging as one of the most important predictors of cancer progression (Lanitis, et al., Annals of Oncology 28, xii18-xii32 (2017)). The presence of tumor-infiltrating lymphocytes (TILs) has retrospectively shown to be correlated to overall patient survival (Galon, et al., Science 313, 1960-1964 (2006)). Compared with the PE-OvaI/II control, a significant increase in the percentage CD4⁺ (three-fold) and CD8⁺ (two-fold) T cells were observed among all cells in the tumors of Man-OVAI/II immunized mice (FIG. 6H). A significant increase in the percentage of CD4⁺ (2.3-fold) and CD8⁺ (1.4-fold) T cells was observed among CD3⁺ T cells in the tumors of the VLP-Man-OVAI/II group compared to the PE-OvaI/II group (FIG. 6I).

Melanoma is a highly metastatic and recurrent disease with approximately one-third of patients experiencing disease recurrence (Tas, Journal of Oncology 2012, 1-9 (2012)). Therefore, protecting against melanoma recurrence is extremely important. To address this challenge, surviving mice from the Man-OvaI/II cohort were re-challenged with B16F10-Ova on Day 28 (FIG. 6J). The Man-OvaI/II cohort was protected against B16F10-Ova re-challenge, while naïve mice rapidly developed tumors (FIG. 6K). This immune protection following therapeutic vaccine administration indicates the establishment of long-term memory T cells that can combat disease recurrence. Collectively, these results demonstrate that immunization with Man-OvaI/II generates robust tumor antigen-specific CD4⁺ and CD8⁺ T cells capable of tumor infiltration with potent anti-tumor responses.

DC-SIGN Engagement Induces a Shift from Antibody Production Towards Cellular Immunity

Humoral responses were also investigated by analyzing antibody profiles of mice immunized with VLPs. OVA(323-339)-specific antibodies were evaluated on day 21 following 3 immunizations with Man-OvaI/II or PE-OvaI/II (6 days following the last dose). Serum ELISA analyses showed that Man-OvaI/II-immunized mice produced lower OVA(323-339)-specific IgG antibody titers than mice immunized with PE-OvaI/II (FIG. 6L-6M). This decrease in antibody production is consistent with the belief that Man-OvaI/II biases the immune response towards cellular immunity (Th1) and away from antibody production (Th2). Furthermore, PE-OvaI/II and Man-OvaI/II immunization induced distinct antibody profiles-PE-OvaI/II primarily induced IgG1-type antibodies while Man-OvaI/II primarily induced IgG2b/c antibodies (FIG. 6L-6M). IgG2c/IgG1 ratios were evaluated, where higher ratios are indicative of a Th1-type immune response. Mice immunized with Man-OvaI/II displayed an antibody subclass IgG2c/IgG1 ratio greater than 1, while mice immunized with PE-OvaI/II displayed a ratio less than 1 (FIG. 6N). These data are consistent with the increased T cell responses observed, confirming a shift from humoral immunity to cellular immunity due to Man-OvaI/II engagement of DC-SIGN.

### Man-OVAI/II Serves as a Self-Adjuvanting Vaccine

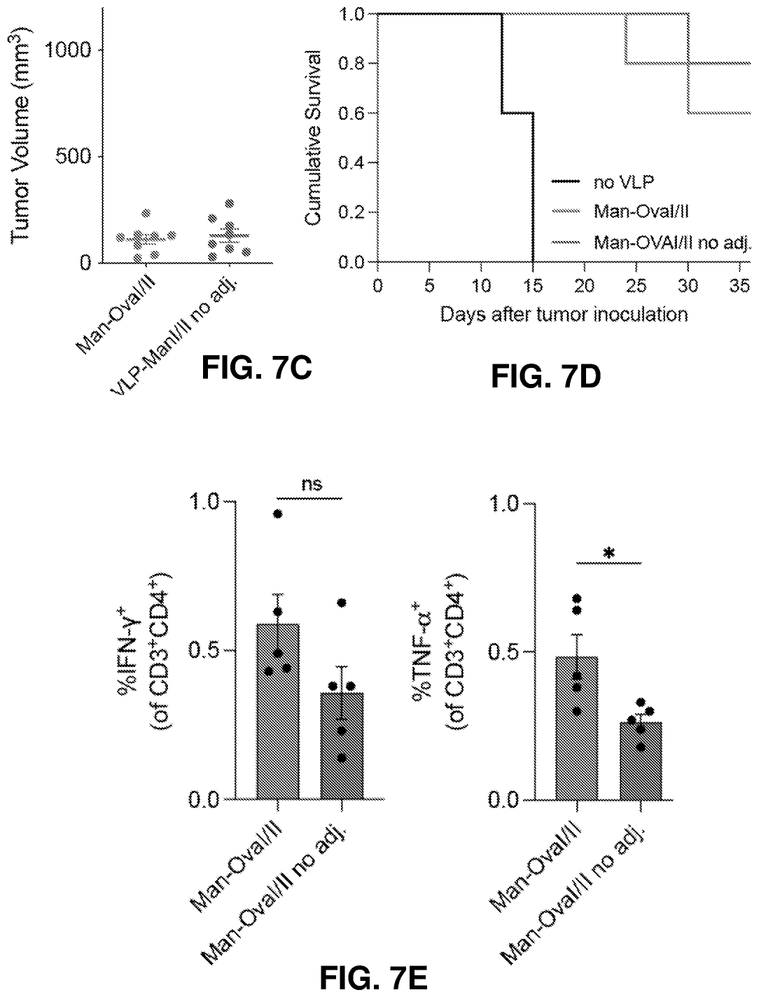

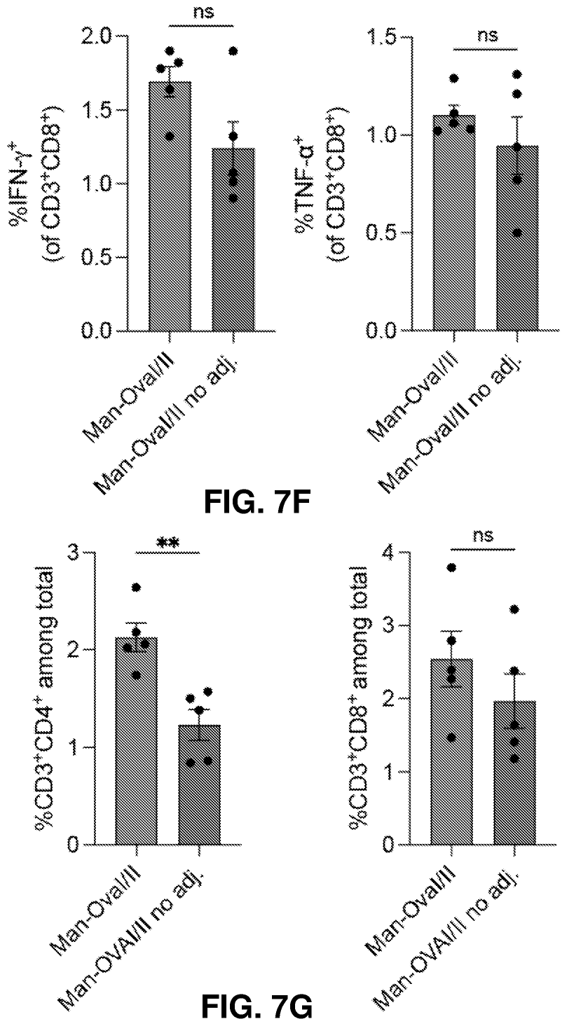

Having demonstrated that Man-OvaI/II is capable of inducing tumor-antigen specific CD4⁺ and CD8⁺ T cells towards anti-tumor immunity, experiments were designed to assess whether this synergistic engagement of DC-SIGN and TLR7 alone was sufficient to induce robust immune responses. To this end, mice inoculated with B16F10-OVA tumor cells were immunized with Man-OvaI/II and anti-PD-1 with and without 2′3′-cGAMP, which had previously been used as a pro-inflammatory adjuvant (FIG. 7A). No significant difference in tumor growth or animal survival was observed (FIG. 7B-7D). Immune cell analysis showed a slight decrease in the percentage of antigen-specific CD4⁺ T cells and no significant difference in the percentage of antigen-specific CD8⁺ T cells (FIG. 7E-7F). The same trends were observed in the tumor, with a slight decrease in the percentage of tumor-infiltrating CD4⁺ T cells and no significant decrease in the percentage of tumor-infiltrating CD8⁺ T cells (FIG. 7G). These data indicate that Man-OvaI/II can serve as a self-adjuvanting cancer vaccine due to the immunostimulatory signaling achieved via combinatorial engagement of DC-SIGN and TLR7.

### DISCUSSION

Many cancer immunotherapies currently rely on ex vivo engineering of DCs or T cells to present or recognize/against tumor antigens. Manipulation of autologous DCs and T cells involves complex, lengthy, and expensive procedures as well as presents significant limitations—low T cell persistence and poor tumor infiltration—resulting in success only in hematologic cancers and not in solid tumors (Rafiq, et al., Nature Reviews Clinical Oncology 17, 147-167 (2020)). Other immunotherapies rely upon direct injections of drugs in the tumor to achieve local anti-tumor stimulation but require tumor accessibility and fail to protect against metastases or disease recurrence. Therefore, the development of effective cancer vaccines that overcome the current limitations will require strategies that enable the in vivo priming of T cells against tumor antigens and infiltration of the TME (Saxena, et al., Nature Reviews Cancer 21, 360-378 (2021)). Importantly, CD4⁺ and CD8⁺ T cell priming relies on efficient antigen presentation by activated DCs—a persistent challenge when trying to achieve immunity against endogenous, chronic tumor antigens (van der Burg, et al., Nature Reviews Cancer 16, 219-233 (2016)). Achieving the desired T cell polarization requires activation of distinct signaling pathways that lead to upregulation of DC costimulatory molecules (CD80, CD86, CD40), and release of pro-inflammatory cytokines. To address this challenge, the disclosed compositions leverage PRRs to retrain the immune system to respond to endogenous tumor antigens in the same way that the immune system fights exogenous pathogens.

The Man-Ova/II VLP platform is uniquely designed to leverage lectin-glycan recognition to redirect immune responses against tumor antigens. Functionalization of the self-assembled VLP PP7 with exogenous glycoligands and tumor antigens yielded the vaccine candidate VLP-Man-OvaI/II. The use of a synthetically accessible glycoligand to target the lectin DC-SIGN facilitated efficient DC-SIGN engagement and signaling, without requiring isolation of native and otherwise prohibitively complex carbohydrates. Due to the size of the VLP, Man-OvaI/II trafficked directly to the immune cell-rich LNs following s.c. administration. DC-SIGN-mediated endocytosis facilitated highly efficient uptake of Man-OvaI/II and enabled TLR7 recognition of encapsulated ssRNAs, eliciting combinatorial signaling through DC-SIGN and TLR7 co-stimulation. Man-OvaI/II induced higher levels of DC activation as compared to control particles lacking either glycoligands or ssRNAs, demonstrating the synergy of DC-SIGN and TLR7 co-engagement. The signaling induced by DC-SIGN and TLR7 co-engagement led to a Th1-type cellular immune response—characterized by pro-inflammatory IFN-γ and TNF-α cytokine secretion—that is important for anti-tumor immunity. Additionally, cleavage of the cathepsin-sensitive epitopes within the endosome released tumor antigens for loading onto MHCI/II complexes. Efficient presentation of tumor antigens on the activated DCs generated tumor antigen-specific CD4⁺ and CD8⁺ T cells capable of tumor infiltration. The results demonstrated that induction of Th1-type CD4⁺ helper T cells in addition to CD8+ cytotoxic T cells provides a more robust immune response. Immunization with Man-OvaI/II resulted in significantly enhanced tumor growth inhibition and prolonged survival compared to PE-OvaI/II in a challenging mouse melanoma model. Generation of tumor antigen-specific T cells in vivo appeared to establish immune memory, providing protection against tumor rechallenge. This immune memory plays an important role in providing long-term protection against tumor recurrence following treatment, further enhancing the efficacy of the therapeutic cancer vaccine. Finally, the capacity of Man-OvaI/II was shown to serve as a self-adjuvanting cancer vaccine, preventing the need for highly immunogenic, toxic adjuvants.

Importantly, this therapeutic strategy represents a potential clinical alternative to immunotherapies that manipulate autologous DCs or T cells. The development of an effective DC-targeting cancer vaccine system provides both a more feasible synthetic method as well as a more robust, holistic anti-tumor immune response. The mannosylated VLP platform developed here provides a modular, synthetically accessible and scalable vaccine platform that can effectively generate tumor antigen-specific T cells in vivo. By leveraging the recognition of pathogen-associated molecular patterns, Man-OvaI/II mimics the immunostimulatory signaling of foreign pathogens and induces immune activation against tumor antigens. The straightforward conjugation strategy used for VLP functionalization permits the installation of desired human tumor neoantigens, potentially enabling this platform to be adopted in a clinical setting. These findings exemplify a significant therapeutic advance in the field of cancer vaccines and highlight the potential of the platform in advancing and broadening the application of cancer immunotherapies.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A composition for antigen-specific activation of dendritic cells, comprising
(a) synthetic Virus Like Particles (VLPs);
(b) one or more species of DC-SIGN ligand(s); and
(c) two or more species of peptide antigen(s),
wherein the DC-SIGN ligand(s) and the antigen(s) are displayed at the outer surface of the synthetic VLP.

2. The composition of claim 1, wherein: (a) the composition comprises two or more species of VLPs, wherein each species of VLP comprises a single species of peptide antigen displayed at the outer surface of the VLP; or (b) the composition comprises one or more species of VLPs, wherein each species of VLP comprises two or more species of peptide antigen(s) displayed at the outer surface of the VLP; or (c) the composition further comprises an immunostimulatory agent.

3. (canceled)

4. The composition of claim 1, wherein: (a) each VLP comprises at least one peptide antigen species attached to at least one viral capsid protein within the VLP; (b) the two peptide antigen species are attached to a single viral capsid protein with a VLP; (c) the two peptide antigen species are each independently attached to two different viral capsid proteins within the same VLP; (d) one or both of the peptide antigen species is attached to the viral capsid protein via a polypeptide linker, optionally, wherein the linker polypeptide is a protease cleavable linker polypeptide comprising SEQ ID NO:7; (e) the VLP comprises the Leviviridae PP7 capsid protein; (f) the VLP comprises the capsid protein of SEQ ID NO:1 or SEQ ID NO:3; and/or (g) the DC-SIGN ligand(s) comprise aryl-mannose, aryl-fucose, or combinations thereof, and optionally is the ligand of Formula I.

5. (canceled)

6. The composition of claim 1, wherein the two peptide antigen species are each independently attached to two different viral capsid proteins within the same VLP.

7. (canceled)

8. (canceled)

9. (canceled)

10. (canceled)

11. (canceled)

12. (canceled)

13. (canceled)

14. (canceled)

15. The composition of claim 14, wherein the immunostimulatory agent is a Toll-like receptor (TLR) agonist, optionally, wherein the TLR agonist is a nucleic acid, preferably a microbial nucleic acid, more preferably a bacterial nucleic acid, optionally, wherein the TLR agonist is encapsidated within the VLP.

16. (canceled)

17. (canceled)

18. The composition of claim 1, wherein: (a) at least one species of the peptide antigen comprises an MHC class I epitope, or an MHC class II epitope, or (b) the two or more species of antigen comprises an MHC class I epitope and an MHC class II epitope.

19. (canceled)

20. The composition of claim 1, wherein at least one of the peptide antigen species is a tumor associated antigen (TAA), optionally wherein the two species of peptide antigen comprise two different tumor associated antigens, wherein the one or more tumor associated antigen(s) are individually selected from the group consisting of an oncogene expression product, an alternatively spliced protein, a mutated gene product, an over-expressed gene product, an aberrantly expressed gene product, an antigen produced by an oncogenic virus, an oncofetal antigen, a protein with altered cell surface glycolipids, and combinations thereof, optionally, (a) wherein the at least one tumor antigen is a tumor specific antigen associated with a cancer selected from the group consisting of bladder, brain, breast, cervical, colo-rectal, esophageal, kidney, liver, lung, naso-pharangeal, pancreatic, prostate, skin, stomach, uterine, ovarian, testicular and hematologic cancer and/or (b) wherein at least one antigen is a personalized neoantigen isolated from a subject, preferably wherein the two species of peptide antigen comprises two different personalized neoantigens isolated from a subject.

21. (canceled)

22. (canceled)

23. (canceled)

24. The composition of claim 1, wherein at least one peptide antigen is derived from, or raises an immune response to a virus, a bacterium, a fungi, a protozoan, a nematode, a plant, or an insect; optionally, wherein the antigen is derived from a pathogenic virus; optionally, wherein the viral antigen is derived from a virus selected from the group consisting of a Coronavirus, Influenza virus, Ebola virus, Zika Virus, and combinations thereof.

25. (canceled)

26. (canceled)

27. The composition of claim 1, wherein the VLPs comprise one or more additional active agents,
wherein the additional active agents are encapsulated within, or displayed upon the surface of the VLPs; optionally, wherein the VLPs encapsulate one or more additional active agents selected from the group consisting of a therapeutic agent, a prophylactic agent, a diagnostic agent, and an adjuvant.

28. (canceled)

29. A pharmaceutical composition or vaccine composition comprising the composition of claim 1, and a pharmaceutically acceptable excipient for administration to a subject in vivo.

30. The composition of claim 29, further comprising one or more additional active agents, wherein the additional active agents are not encapsulated within, or displayed upon the surface of the VLPs; optionally, wherein the active agent is selected from the group consisting of a therapeutic agent, a prophylactic agent, a diagnostic agent, and an adjuvant.

31. (canceled)

32. The composition of claim 30, wherein the composition is a pharmaceutical composition and the active agent is a chemotherapeutic agent, optionally, wherein the active agent is selected from the group consisting of a checkpoint inhibitor and a STING agonist, or both a checkpoint inhibitor and a STING agonist.

33. (canceled)

34. The vaccine comprising the composition of claim 29 in an amount effective to activate dendritic cells and stimulate an immune response to the antigen in a subject, optionally further comprising an adjuvant.

35. A method of generating an immune response to an antigen in a subject, comprising administering the composition of claim 1, to the subject in an amount effective to activate dendritic cells and stimulate an immune response to the antigen in the subject.

36. The method of claim 35, wherein: (a) the subject has, or is at risk of having an infectious disease, wherein the antigen is derived from or stimulates an immune response to a pathogen associated with the disease, and wherein the antigen stimulates an immune response to the pathogen in the subject; or (b) the subject has, or is at risk of having cancer, wherein the antigen is a tumor antigen, and
wherein the antigen stimulates an immune response to the tumor antigen in the subject.

37. (canceled)

38. The method of claim 37, wherein the cancer is selected from the group consisting of bladder, brain, breast, cervical, colo-rectal, esophageal, kidney, liver, lung, nasopharangeal, pancreatic, prostate, skin, stomach, uterine, ovarian, testicular and hematologic cancer.

39. The method of claim 35: (a) wherein the immune response is a T-helper 1 (TH1)-type immune response to the antigen; (b) further comprising administering one or more additional active agents to the subject; optionally, wherein the active agent is selected from the group consisting of a therapeutic agent, a prophylactic agent, a diagnostic agent, and an adjuvant, optionally, wherein the active agent is a chemotherapeutic agent.

40. (canceled)

41. (canceled)

42. (canceled)

43. The method of claim 42, wherein the active agent is selected from the group consisting of a checkpoint inhibitor and a STING agonist, or both a checkpoint inhibitor and a STING agonist, optionally, wherein (a) the checkpoint inhibitor is a PD-1 inhibitor, (b) the PD-1 inhibitor is an anti-PD1 antibody or (c) the STING agonist is a 2/3/-cGAMP.

44. (canceled)

45. (canceled)

46. (canceled)

47. The method of claim 40, wherein administering the additional active agent to the subject together with the composition of VLPs provides more effective immunity to the antigen than the response raised to the antigen when the additional active agent or the composition of VLPs are administered to the subject alone.

48. (canceled)

49. A method of synthesizing a DC-SIGN ligand of claim 1, comprising 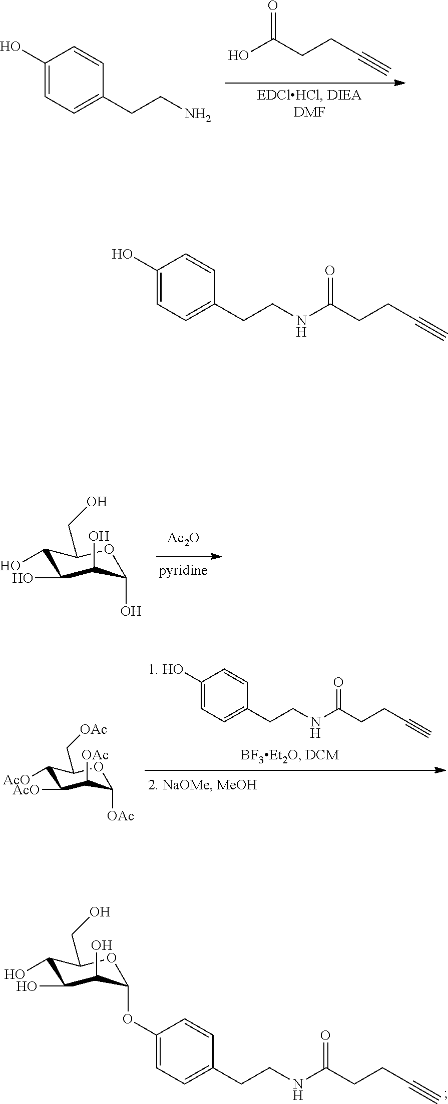
or an aryl-bearing core displaying monosaccharides or oligosaccharides or glycans, comprising 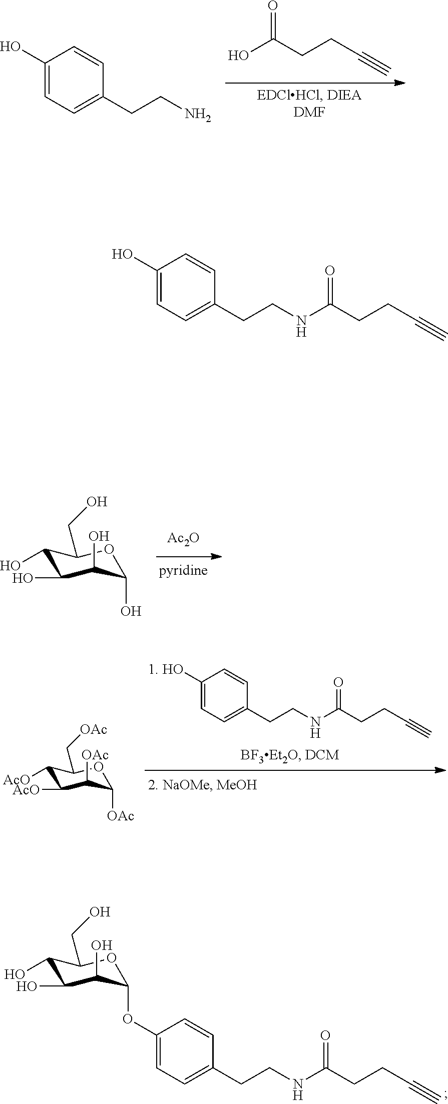
modified by substituting the mannopyranose with the alternative monosaccharide(s) or oligosaccharide(s) or glycan(s).

50. (canceled)


SEQUENCE LISTING (11 sequences)

SEQ ID NO: 1 - protein, 127 aa, synthetic construct
SKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGR

SEQ ID NO: 2 - protein, 4 aa, synthetic construct
AYGG

SEQ ID NO: 3 - protein, 258 aa, synthetic construct
SKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGRAYGGSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGR

SEQ ID NO: 4 - protein, 8 aa, synthetic construct
GGASESGA

SEQ ID NO: 5 - protein, 266 aa, synthetic construct
SKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGRAYGGSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGRGGASESGA

SEQ ID NO: 6 - protein, 7 aa, synthetic construct
DGSPLEF

SEQ ID NO: 7 - protein, 15 aa, synthetic construct
GGASESGADGSPLEF

SEQ ID NO: 8 - protein, 4 aa, synthetic construct
GFLG

SEQ ID NO: 9 - protein, 4 aa, synthetic construct
ALAL

SEQ ID NO: 10 - protein, 8 aa, synthetic construct
SIINFEKL

SEQ ID NO: 11 - protein, 17 aa, synthetic construct
ISQAVHAAHAEINEAGR